SEQUENCE LISTING (709 sequences)

SEQ ID NO: 1 - protein, 9 aa, Artificial Sequence
  CS9.3 EGFR peptide with C797S
QLMPFGSLL

SEQ ID NO: 2 - protein, 11 aa, Artificial Sequence
  CS11.6 EGFR peptide with C797S mutation
LMPFGSLLDYV

SEQ ID NO: 3 - protein, 10 aa, Artificial Sequence
  GD10.3 KRAS peptide with G12D mutation
KLVVVGADGV

SEQ ID NO: 4 - protein, 10 aa, Artificial Sequence
  GV10.3 KRAS peptide with G12V mutation
KLVVVGAVGV

SEQ ID NO: 5 - protein, 10 aa, Artificial Sequence
  GC10.3 KRAS peptide with G12C mutation
KLVVVGACGV

SEQ ID NO: 6 - protein, 5 aa, Homo sapiens
DSVNN

SEQ ID NO: 7 - protein, 6 aa, Homo sapiens
SSVSVY

SEQ ID NO: 8 - protein, 7 aa, Homo sapiens
TISGNEY

SEQ ID NO: 9 - protein, 6 aa, Homo sapiens
SSVPPY

SEQ ID NO: 10 - protein, 6 aa, Homo sapiens
VSPFSN

SEQ ID NO: 11 - protein, 5 aa, Homo sapiens
TSINN

SEQ ID NO: 12 - protein, 6 aa, Homo sapiens
SSNFYA

SEQ ID NO: 13 - protein, 6 aa, Homo sapiens
DRGSQS

SEQ ID NO: 14 - protein, 7 aa, Homo sapiens
TSESDYY

SEQ ID NO: 15 - protein, 6 aa, Homo sapiens
TSGFNG

SEQ ID NO: 16 - protein, 6 aa, Homo sapiens
NYSPAY

SEQ ID NO: 17 - protein, 7 aa, Homo sapiens
TRDTTYY

SEQ ID NO: 18 - protein, 7 aa, Homo sapiens
TSDPSYG

SEQ ID NO: 19 - protein, 6 aa, Homo sapiens
TSGFYG

SEQ ID NO: 20 - protein, 5 aa, Homo sapiens
SIFNT

SEQ ID NO: 21 - protein, 6 aa, Homo sapiens
YSGSPE

SEQ ID NO: 22 - protein, 6 aa, Homo sapiens
SSYSPS

SEQ ID NO: 23 - protein, 6 aa, Homo sapiens
NSAFQY

SEQ ID NO: 24 - protein, 6 aa, Homo sapiens
DSASNY

SEQ ID NO: 25 - protein, 6 aa, Homo sapiens
NSASDY

SEQ ID NO: 26 - protein, 6 aa, Homo sapiens
YGGTVN

SEQ ID NO: 27 - protein, 6 aa, Homo sapiens
NTAFDY

SEQ ID NO: 28 - protein, 7 aa, Homo sapiens
TSDQSYG

SEQ ID NO: 29 - protein, 6 aa, Homo sapiens
DSAIYN

SEQ ID NO: 30 - protein, 6 aa, Homo sapiens
YGATPY

SEQ ID NO: 31 - protein, 7 aa, Homo sapiens
TSENNYY

SEQ ID NO: 32 - protein, 6 aa, Homo sapiens
DRVSQS

SEQ ID NO: 33 - protein, 6 aa, Homo sapiens
ATGYPS

SEQ ID NO: 34 - protein, 5 aa, Homo sapiens
SGHNT

SEQ ID NO: 35 - protein, 5 aa, Homo sapiens
SGHDT

SEQ ID NO: 36 - protein, 5 aa, Homo sapiens
SQVTM

SEQ ID NO: 37 - protein, 5 aa, Homo sapiens
SGHDY

SEQ ID NO: 38 - protein, 5 aa, Homo sapiens
KGHSH

SEQ ID NO: 39 - protein, 6 aa, Homo sapiens
DFQATT

SEQ ID NO: 40 - protein, 6 aa, Homo sapiens
GTSNPN

SEQ ID NO: 41 - protein, 5 aa, Homo sapiens
SGHDN

SEQ ID NO: 42 - protein, 5 aa, Homo sapiens
MNHEY

SEQ ID NO: 43 - protein, 5 aa, Homo sapiens
SGHVS

SEQ ID NO: 44 - protein, 5 aa, Homo sapiens
SNHLY

SEQ ID NO: 45 - protein, 5 aa, Homo sapiens
LNHNV

SEQ ID NO: 46 - protein, 5 aa, Homo sapiens
SGHAT

SEQ ID NO: 47 - protein, 5 aa, Homo sapiens
LNHDA

SEQ ID NO: 48 - protein, 5 aa, Homo sapiens
ENHRY

SEQ ID NO: 49 - protein, 5 aa, Homo sapiens
WSHSY

SEQ ID NO: 50 - protein, 5 aa, Homo sapiens
LGHNT

SEQ ID NO: 51 - protein, 5 aa, Homo sapiens
LGHDT

SEQ ID NO: 52 - protein, 5 aa, HoJo virus
MDHEN

SEQ ID NO: 53 - protein, 5 aa, Homo sapiens
MNHNS

SEQ ID NO: 54 - protein, 5 aa, Homo sapiens
MGHRA

SEQ ID NO: 55 - protein, 5 aa, Homo sapiens
SGHYY

SEQ ID NO: 56 - protein, 5 aa, Homo sapiens
SEHNR

SEQ ID NO: 57 - protein, 5 aa, Homo sapiens
PGHNT

SEQ ID NO: 58 - protein, 5 aa, Homo sapiens
SVFSS

SEQ ID NO: 59 - protein, 5 aa, Homo sapiens
SGHRS

SEQ ID NO: 60 - protein, 5 aa, Homo sapiens
MRHNA

SEQ ID NO: 61 - protein, 5 aa, Homo sapiens
PRHDT

SEQ ID NO: 62 - protein, 5 aa, Homo sapiens
MDHEY

SEQ ID NO: 63 - protein, 5 aa, Homo sapiens
KGHSR

SEQ ID NO: 64 - protein, 6 aa, Homo sapiens
DSSSTY

SEQ ID NO: 65 - protein, 5 aa, Homo sapiens
KGHDR

SEQ ID NO: 66 - protein, 5 aa, Homo sapiens
IPSGT

SEQ ID NO: 67 - protein, 8 aa, Homo sapiens
YLSGSTLV

SEQ ID NO: 68 - protein, 5 aa, Homo sapiens
GLKNN

SEQ ID NO: 69 - protein, 8 aa, Homo sapiens
YTSAATLV

SEQ ID NO: 70 - protein, 7 aa, Homo sapiens
MTFSENT

SEQ ID NO: 71 - protein, 7 aa, Homo sapiens
IRSNERE

SEQ ID NO: 72 - protein, 7 aa, Homo sapiens
MTLNGDE

SEQ ID NO: 73 - protein, 6 aa, Homo sapiens
IYSNGD

SEQ ID NO: 74 - protein, 8 aa, Homo sapiens
QEAYKQQN

SEQ ID NO: 75 - protein, 6 aa, Homo sapiens
NVLDGL

SEQ ID NO: 76 - protein, 7 aa, Homo sapiens
IRENEKE

SEQ ID NO: 77 - protein, 8 aa, Homo sapiens
RNSFDEQN

SEQ ID NO: 78 - protein, 5 aa, Homo sapiens
GLTSN

SEQ ID NO: 79 - protein, 7 aa, Homo sapiens
TISGTDY

SEQ ID NO: 80 - protein, 7 aa, Homo sapiens
IRSNVGE

SEQ ID NO: 81 - protein, 6 aa, Homo sapiens
LQKENI

SEQ ID NO: 82 - protein, 8 aa, Homo sapiens
QGSYDQQN

SEQ ID NO: 83 - protein, 6 aa, Homo sapiens
DALDGL

SEQ ID NO: 84 - protein, 7 aa, Homo sapiens
LYKAGEL

SEQ ID NO: 85 - protein, 6 aa, Homo sapiens
IFSNGE

SEQ ID NO: 86 - protein, 7 aa, Homo sapiens
IFSNMDM

SEQ ID NO: 87 - protein, 6 aa, Homo sapiens
FQDESV

SEQ ID NO: 88 - protein, 6 aa, Homo sapiens
SVGAGI

SEQ ID NO: 89 - protein, 7 aa, Homo sapiens
SNEGSKA

SEQ ID NO: 90 - protein, 7 aa, Homo sapiens
ANQGSEA

SEQ ID NO: 91 - protein, 6 aa, Homo sapiens
SVGEGT

SEQ ID NO: 92 - protein, 6 aa, Homo sapiens
SASEGT

SEQ ID NO: 93 - protein, 6 aa, Homo sapiens
SYDVKM

SEQ ID NO: 94 - protein, 6 aa, Homo sapiens
FYNNEI

SEQ ID NO: 95 - protein, 4 aa, Homo sapiens
HISR

SEQ ID NO: 96 - protein, 8 aa, Homo sapiens
YTTGATLV

SEQ ID NO: 97 - protein, 6 aa, Homo sapiens
TYSSGN

SEQ ID NO: 98 - protein, 7 aa, Homo sapiens
IRSNMDK

SEQ ID NO: 99 - protein, 8 aa, Homo sapiens
YFSGDPLV

SEQ ID NO: 100 - protein, 7 aa, Homo sapiens
ATKADDK

SEQ ID NO: 101 - protein, 6 aa, Homo sapiens
YYREEE

SEQ ID NO: 102 - protein, 6 aa, Homo sapiens
YYEEEE

SEQ ID NO: 103 - protein, 6 aa, Homo sapiens
FNNNVP

SEQ ID NO: 104 - protein, 5 aa, Homo sapiens
SVGIG

SEQ ID NO: 105 - protein, 6 aa, Homo sapiens
FVKESK

SEQ ID NO: 106 - protein, 6 aa, Homo sapiens
FNYEAQ

SEQ ID NO: 107 - protein, 6 aa, Homo sapiens
YYDKDF

SEQ ID NO: 108 - protein, 6 aa, Homo sapiens
FQNNGV

SEQ ID NO: 109 - protein, 6 aa, Homo sapiens
SQIVND

SEQ ID NO: 110 - protein, 6 aa, Homo sapiens
SYGVKD

SEQ ID NO: 111 - protein, 6 aa, Homo sapiens
SAAADI

SEQ ID NO: 112 - protein, 6 aa, Homo sapiens
FRNRAP

SEQ ID NO: 113 - protein, 6 aa, Homo sapiens
YYGEEE

SEQ ID NO: 114 - protein, 6 aa, Homo sapiens
YNNKEL

SEQ ID NO: 115 - protein, 6 aa, Homo sapiens
SMNVEV

SEQ ID NO: 116 - protein, 6 aa, Homo sapiens
YSYEKL

SEQ ID NO: 117 - protein, 6 aa, Homo sapiens
FQNEAQ

SEQ ID NO: 118 - protein, 7 aa, Homo sapiens
VVTGGEV

SEQ ID NO: 119 - protein, 6 aa, Homo sapiens
SQVVND

SEQ ID NO: 120 - protein, 6 aa, Homo sapiens
IRPDVS

SEQ ID NO: 121 - protein, 8 aa, Homo sapiens
QGSYDEQN

SEQ ID NO: 122 - protein, 7 aa, Homo sapiens
IQSSQRE

SEQ ID NO: 123 - protein, 8 aa, Homo sapiens
YFSGDTLV

SEQ ID NO: 124 - protein, 6 aa, Homo sapiens
YFSETQ

SEQ ID NO: 125 - protein, 6 aa, Homo sapiens
SNTAGT

SEQ ID NO: 126 - protein, 6 aa, Homo sapiens
FYEKMQ

SEQ ID NO: 127 - protein, 6 aa, Homo sapiens
SFDVKD

SEQ ID NO: 128 - protein, 6 aa, Homo sapiens
FNNKEL

SEQ ID NO: 129 - protein, 7 aa, Homo sapiens
SNEGSRA

SEQ ID NO: 130 - protein, 10 aa, Homo sapiens
AVDNYGQNFV

SEQ ID NO: 131 - protein, 10 aa, Homo sapiens
AVGNYGQNFV

SEQ ID NO: 132 - protein, 10 aa, Homo sapiens
AVDSYGQNFV

SEQ ID NO: 133 - protein, 10 aa, Homo sapiens
AADNYGQNFV

SEQ ID NO: 134 - protein, 10 aa, Homo sapiens
AVDDYGQNFV

SEQ ID NO: 135 - protein, 12 aa, Homo sapiens
AVTFTGGGNKLT

SEQ ID NO: 136 - protein, 10 aa, Homo sapiens
AVNNYGQNFV

SEQ ID NO: 137 - protein, 13 aa, Homo sapiens
IVNWGSNSGYALN

SEQ ID NO: 138 - protein, 10 aa, Homo sapiens
AEDNYGQNFV

SEQ ID NO: 139 - protein, 10 aa, Homo sapiens
AVVNYGQNFV

SEQ ID NO: 140 - protein, 10 aa, Homo sapiens
AVDIYGQNFV

SEQ ID NO: 141 - protein, 15 aa, Homo sapiens
AVSEMNYGGSQGNLI

SEQ ID NO: 142 - protein, 8 aa, Homo sapiens
AVEGYKLS

SEQ ID NO: 143 - protein, 10 aa, Homo sapiens
VAMNRDDKII

SEQ ID NO: 144 - protein, 10 aa, Homo sapiens
AVENYGQNFV

SEQ ID NO: 145 - protein, 12 aa, Homo sapiens
ATVSTSGTYKYI

SEQ ID NO: 146 - protein, 11 aa, Homo sapiens
AVSDTGFQKLV

SEQ ID NO: 147 - protein, 10 aa, Homo sapiens
AVDYYGQNFV

SEQ ID NO: 148 - protein, 10 aa, Homo sapiens
AVYNYGQNFV

SEQ ID NO: 149 - protein, 10 aa, Homo sapiens
AGDNYGQNFV

SEQ ID NO: 150 - protein, 10 aa, Homo sapiens
AVDTYGQNFV

SEQ ID NO: 151 - protein, 10 aa, Homo sapiens
AVANYGQNFV

SEQ ID NO: 152 - protein, 10 aa, Homo sapiens
AFMSGYSTLT

SEQ ID NO: 153 - protein, 13 aa, Homo sapiens
AVNLGGGGADGLT

SEQ ID NO: 154 - protein, 14 aa, Homo sapiens
AVEGYSGAGSYQLT

SEQ ID NO: 155 - protein, 10 aa, Homo sapiens
AVDHYGQNFV

SEQ ID NO: 156 - protein, 10 aa, Homo sapiens
AVEPHNARLM

SEQ ID NO: 157 - protein, 10 aa, Homo sapiens
AVDKYGQNFV

SEQ ID NO: 158 - protein, 12 aa, Homo sapiens
AYRSAVTGNQFY

SEQ ID NO: 159 - protein, 10 aa, Homo sapiens
AVHNYGQNFV

SEQ ID NO: 160 - protein, 12 aa, Homo sapiens
AVDLNSGYSTLT

SEQ ID NO: 161 - protein, 11 aa, Homo sapiens
AVSDPGDEKLT

SEQ ID NO: 162 - protein, 13 aa, Homo sapiens
ATVQNTGTASKLT

SEQ ID NO: 163 - protein, 9 aa, Homo sapiens
ALGTEMTRS

SEQ ID NO: 164 - protein, 12 aa, Homo sapiens
ALSDSEGAQKLV

SEQ ID NO: 165 - protein, 8 aa, Homo sapiens
AVDGQKLL

SEQ ID NO: 166 - protein, 11 aa, Homo sapiens
AMREGGDDKII

SEQ ID NO: 167 - protein, 14 aa, Homo sapiens
IVRVASGGGADGLT

SEQ ID NO: 168 - protein, 8 aa, Homo sapiens
ALRRLQNY

SEQ ID NO: 169 - protein, 12 aa, Homo sapiens
AVLPQGGSEKLV

SEQ ID NO: 170 - protein, 10 aa, Homo sapiens
AVDNRGQNFV

SEQ ID NO: 171 - protein, 9 aa, Homo sapiens
ALYNFNKFY

SEQ ID NO: 172 - protein, 11 aa, Homo sapiens
AGQLTLATQAN

SEQ ID NO: 173 - protein, 10 aa, Homo sapiens
AVDSCGQNFV

SEQ ID NO: 174 - protein, 9 aa, Homo sapiens
AGITMVRIL

SEQ ID NO: 175 - protein, 12 aa, Homo sapiens
AQGSLALATQAN

SEQ ID NO: 176 - protein, 12 aa, Homo sapiens
ACFNSNSGYALN

SEQ ID NO: 177 - protein, 12 aa, Homo sapiens
AMRASGGYQKVT

SEQ ID NO: 178 - protein, 13 aa, Homo sapiens
VVSRIMEEAKEIS

SEQ ID NO: 179 - protein, 10 aa, Homo sapiens
AVSGYNNDMR

SEQ ID NO: 180 - protein, 10 aa, Homo sapiens
AVGTGANNLF

SEQ ID NO: 181 - protein, 8 aa, Homo sapiens
AGLWSEFC

SEQ ID NO: 182 - protein, 7 aa, Homo sapiens
AASIMTC

SEQ ID NO: 183 - protein, 12 aa, Homo sapiens
AMDVYNQGGKLI

SEQ ID NO: 184 - protein, 10 aa, Homo sapiens
VVSGVGQNFV

SEQ ID NO: 185 - protein, 10 aa, Homo sapiens
AVDNCGQNFV

SEQ ID NO: 186 - protein, 10 aa, Homo sapiens
AVDDHGQNFV

SEQ ID NO: 187 - protein, 10 aa, Homo sapiens
AMDNYGQNFV

SEQ ID NO: 188 - protein, 12 aa, Homo sapiens
AVTFAGGGNKLT

SEQ ID NO: 189 - protein, 12 aa, Homo sapiens
AVAFTGGGNKLT

SEQ ID NO: 190 - protein, 10 aa, Homo sapiens
AVSLNDYKLS

SEQ ID NO: 191 - protein, 12 aa, Homo sapiens
AEISYSSASKII

SEQ ID NO: 192 - protein, 10 aa, Homo sapiens
TVDNYGQNFV

SEQ ID NO: 193 - protein, 10 aa, Homo sapiens
AVDNYSQNFV

SEQ ID NO: 194 - protein, 10 aa, Homo sapiens
AVDNHGQNFV

SEQ ID NO: 195 - protein, 8 aa, Homo sapiens
AYRSHDMR

SEQ ID NO: 196 - protein, 10 aa, Homo sapiens
AYGGGSEKLV

SEQ ID NO: 197 - protein, 12 aa, Homo sapiens
AVTSTGGGNKLT

SEQ ID NO: 198 - protein, 12 aa, Homo sapiens
AVTLTGGGNKLT

SEQ ID NO: 199 - protein, 11 aa, Homo sapiens
APPSGSARQLT

SEQ ID NO: 200 - protein, 10 aa, Homo sapiens
AVNSYGQNFV

SEQ ID NO: 201 - protein, 10 aa, Homo sapiens
AVNDYGQNFV

SEQ ID NO: 202 - protein, 10 aa, Homo sapiens
AAVNYGQNFV

SEQ ID NO: 203 - protein, 10 aa, Homo sapiens
AANNYGQNFV

SEQ ID NO: 204 - protein, 11 aa, Homo sapiens
AVNRNTGNQFY

SEQ ID NO: 205 - protein, 11 aa, Homo sapiens
VVSAKEAKEIS

SEQ ID NO: 206 - protein, 16 aa, Homo sapiens
VVSAEGRQRLNPGEAI

SEQ ID NO: 207 - protein, 10 aa, Homo sapiens
ATGSNDYKLS

SEQ ID NO: 208 - protein, 11 aa, Homo sapiens
ATDGRGSYIPT

SEQ ID NO: 209 - protein, 11 aa, Homo sapiens
ATDEDSSYKLI

SEQ ID NO: 210 - protein, 10 aa, Homo sapiens
AVSDSNYQLI

SEQ ID NO: 211 - protein, 13 aa, Homo sapiens
AYRSAGGATNKLI

SEQ ID NO: 212 - protein, 12 aa, Homo sapiens
AFMKHSGVNDMR

SEQ ID NO: 213 - protein, 13 aa, Homo sapiens
IVSWGSNSGYALN

SEQ ID NO: 214 - protein, 9 aa, Homo sapiens
ICSGNTPLV

SEQ ID NO: 215 - protein, 13 aa, Homo sapiens
IANWGSNSGYALN

SEQ ID NO: 216 - protein, 12 aa, Homo sapiens
AVTYTGGGNKLT

SEQ ID NO: 217 - protein, 12 aa, Homo sapiens
AVTFMGGGNKLT

SEQ ID NO: 218 - protein, 12 aa, Homo sapiens
AVTFKGGGNKLT

SEQ ID NO: 219 - protein, 10 aa, Homo sapiens
AVSDRGETSW

SEQ ID NO: 220 - protein, 11 aa, Homo sapiens
AVSDAGFQKLV

SEQ ID NO: 221 - protein, 12 aa, Homo sapiens
AATFTGGGNKLT

SEQ ID NO: 222 - protein, 15 aa, Homo sapiens
AVSGMNYGGSQGNLI

SEQ ID NO: 223 - protein, 10 aa, Homo sapiens
APYTGRRALT

SEQ ID NO: 224 - protein, 10 aa, Homo sapiens
VVDNYGQNFV

SEQ ID NO: 225 - protein, 10 aa, Homo sapiens
VADNYGQNFV

SEQ ID NO: 226 - protein, 10 aa, Homo sapiens
TADNYGQNFV

SEQ ID NO: 227 - protein, 8 aa, Homo sapiens
CGLWSEFC

SEQ ID NO: 228 - protein, 9 aa, Homo sapiens
AWITMVRIL

SEQ ID NO: 229 - protein, 10 aa, Homo sapiens
AVSNYGQNFV

SEQ ID NO: 230 - protein, 9 aa, Homo sapiens
AVSNDYKLS

SEQ ID NO: 231 - protein, 9 aa, Homo sapiens
AVDSGQNFV

SEQ ID NO: 232 - protein, 9 aa, Homo sapiens
AVDNYVRIL

SEQ ID NO: 233 - protein, 10 aa, Homo sapiens
AVDNHSQNFV

SEQ ID NO: 234 - protein, 11 aa, Homo sapiens
AVFGSNTGKLI

SEQ ID NO: 235 - protein, 10 aa, Homo sapiens
ALRSNDYKLS

SEQ ID NO: 236 - protein, 13 aa, Homo sapiens
AVRIAFWGLPESY

SEQ ID NO: 237 - protein, 10 aa, Homo sapiens
ASSLAGYEQY

SEQ ID NO: 238 - protein, 11 aa, Homo sapiens
ASSLGQGKHSF

SEQ ID NO: 239 - protein, 13 aa, Homo sapiens
SVEGGSSGANVLT

SEQ ID NO: 240 - protein, 10 aa, Homo sapiens
ASSSAGYEQY

SEQ ID NO: 241 - protein, 10 aa, Homo sapiens
ASSLAGCEQY

SEQ ID NO: 242 - protein, 13 aa, Homo sapiens
ASTSWGVSYNEQF

SEQ ID NO: 243 - protein, 10 aa, Homo sapiens
ASSLASYEQY

SEQ ID NO: 244 - protein, 10 aa, Homo sapiens
ASSLAGHEQY

SEQ ID NO: 245 - protein, 13 aa, Homo sapiens
ASSPPEGFGNEQF

SEQ ID NO: 246 - protein, 10 aa, Homo sapiens
ASSLTGYEQY

SEQ ID NO: 247 - protein, 13 aa, Homo sapiens
SANLAKSSYNEQF

SEQ ID NO: 248 - protein, 12 aa, Homo sapiens
SAPRDPDADTQY

SEQ ID NO: 249 - protein, 11 aa, Homo sapiens
AWDRTGEVEQY

SEQ ID NO: 250 - protein, 9 aa, Homo sapiens
ASSAGYEQY

SEQ ID NO: 251 - protein, 9 aa, Homo sapiens
AAAWPATSS

SEQ ID NO: 252 - protein, 10 aa, Homo sapiens
ASSMAGYEQY

SEQ ID NO: 253 - protein, 15 aa, Homo sapiens
AWSFHPGLAAYNEQF

SEQ ID NO: 254 - protein, 12 aa, Homo sapiens
ASSQLRGGSPLH

SEQ ID NO: 255 - protein, 13 aa, Homo sapiens
ASSGQGGSNTEAF

SEQ ID NO: 256 - protein, 11 aa, Homo sapiens
ASSLGQGRHSF

SEQ ID NO: 257 - protein, 10 aa, Homo sapiens
ASSLAEDTQY

SEQ ID NO: 258 - protein, 10 aa, Homo sapiens
ASSWAGYEQY

SEQ ID NO: 259 - protein, 11 aa, Homo sapiens
ASSLGRGKHSF

SEQ ID NO: 260 - protein, 10 aa, Homo sapiens
ASSLAGFEQY

SEQ ID NO: 261 - protein, 9 aa, Homo sapiens
ASSLAGEQY

SEQ ID NO: 262 - protein, 11 aa, Homo sapiens
ASSLGQGKRSF

SEQ ID NO: 263 - protein, 11 aa, Homo sapiens
ASSLGQGEHSF

SEQ ID NO: 264 - protein, 10 aa, Homo sapiens
AYSTGYFGYT

SEQ ID NO: 265 - protein, 13 aa, Homo sapiens
SVGGGSSGANVLT

SEQ ID NO: 266 - protein, 10 aa, Homo sapiens
ASSVAGYEQY

SEQ ID NO: 267 - protein, 13 aa, Homo sapiens
SVEGGSPGANVLT

SEQ ID NO: 268 - protein, 11 aa, Homo sapiens
ASSSGQGKHSF

SEQ ID NO: 269 - protein, 15 aa, Homo sapiens
ASSESRYGRDTDTQY

SEQ ID NO: 270 - protein, 17 aa, Homo sapiens
ASSYSYSTGPELNTEAF

SEQ ID NO: 271 - protein, 13 aa, Homo sapiens
SVEGGPSGANVLT

SEQ ID NO: 272 - protein, 10 aa, Homo sapiens
ASSLAGNEQY

SEQ ID NO: 273 - protein, 10 aa, Homo sapiens
ASSLAAYEQY

SEQ ID NO: 274 - protein, 11 aa, Homo sapiens
ASSLSFDSEQY

SEQ ID NO: 275 - protein, 11 aa, Homo sapiens
ASSLSQGKHSF

SEQ ID NO: 276 - protein, 10 aa, Homo sapiens
ASSFAGYEQY

SEQ ID NO: 277 - protein, 10 aa, Homo sapiens
SARQGLTEAF

SEQ ID NO: 278 - protein, 10 aa, Homo sapiens
AWSVLYGTEY

SEQ ID NO: 279 - protein, 10 aa, Homo sapiens
ASSLAGSEQY

SEQ ID NO: 280 - protein, 11 aa, Homo sapiens
SVEGDPLGPTS

SEQ ID NO: 281 - protein, 10 aa, Homo sapiens
ASSLSGYEQY

SEQ ID NO: 282 - protein, 13 aa, Homo sapiens
SVEEGSSGANVLT

SEQ ID NO: 283 - protein, 10 aa, Homo sapiens
ASSLPGYEQY

SEQ ID NO: 284 - protein, 13 aa, Homo sapiens
ATSREGTGENIQY

SEQ ID NO: 285 - protein, 13 aa, Homo sapiens
ASSFSIRASYEQY

SEQ ID NO: 286 - protein, 10 aa, Homo sapiens
ASSLAGDEQY

SEQ ID NO: 287 - protein, 11 aa, Homo sapiens
ASSVASTGELF

SEQ ID NO: 288 - protein, 12 aa, Homo sapiens
ASSLGRGNTEAF

SEQ ID NO: 289 - protein, 12 aa, Homo sapiens
ASSPIRREGEQY

SEQ ID NO: 290 - protein, 15 aa, Homo sapiens
ASFVYSAGDSYNEQF

SEQ ID NO: 291 - protein, 10 aa, Homo sapiens
SARNRVYEQY

SEQ ID NO: 292 - protein, 12 aa, Homo sapiens
ASSLGQGNTEAF

SEQ ID NO: 293 - protein, 11 aa, Homo sapiens
ASSPPGENEQY

SEQ ID NO: 294 - protein, 11 aa, Homo sapiens
ASTDTDLGEQY

SEQ ID NO: 295 - protein, 13 aa, Homo sapiens
SVERGSSGANVLT

SEQ ID NO: 296 - protein, 11 aa, Homo sapiens
ASSLGQGNHSF

SEQ ID NO: 297 - protein, 12 aa, Homo sapiens
ASSLARGNTEAF

SEQ ID NO: 298 - protein, 12 aa, Homo sapiens
AAAWARGNTEAF

SEQ ID NO: 299 - protein, 12 aa, Homo sapiens
ALSDSGTIYEQY

SEQ ID NO: 300 - protein, 17 aa, Homo sapiens
ASSVPLEGGSGPQDTQY

SEQ ID NO: 301 - protein, 11 aa, Homo sapiens
ASSLGQGKYSF

SEQ ID NO: 302 - protein, 12 aa, Homo sapiens
ASGLYNRGNEQF

SEQ ID NO: 303 - protein, 11 aa, Homo sapiens
SVEGGSSGPTS

SEQ ID NO: 304 - protein, 10 aa, Homo sapiens
ASGLAGYEQY

SEQ ID NO: 305 - protein, 12 aa, Homo sapiens
ASSRTRYTDTQY

SEQ ID NO: 306 - protein, 10 aa, Homo sapiens
ASNLAGYEQY

SEQ ID NO: 307 - protein, 13 aa, Homo sapiens
ASASWGVSYNEQF

SEQ ID NO: 308 - protein, 12 aa, Homo sapiens
ASSLGQGETLKL

SEQ ID NO: 309 - protein, 13 aa, Homo sapiens
SVVGGSSGANVLT

SEQ ID NO: 310 - protein, 14 aa, Homo sapiens
SVGANVAGGKETQY

SEQ ID NO: 311 - protein, 13 aa, Homo sapiens
ASSVTGTVNTEAF

SEQ ID NO: 312 - protein, 13 aa, Homo sapiens
SVKGGSSGANVLT

SEQ ID NO: 313 - protein, 13 aa, Homo sapiens
SVEGGSTGANVLT

SEQ ID NO: 314 - protein, 11 aa, Homo sapiens
ASSLGQGIHSF

SEQ ID NO: 315 - protein, 11 aa, Homo sapiens
ASSLGQGKLSF

SEQ ID NO: 316 - protein, 11 aa, Homo sapiens
ASSQDIEVEAF

SEQ ID NO: 317 - protein, 11 aa, Homo sapiens
ASSLRLNTEAF

SEQ ID NO: 318 - protein, 14 aa, Homo sapiens
ASSVEAGVSGNTIY

SEQ ID NO: 319 - protein, 11 aa, Homo sapiens
SVVRQGHYEAF

SEQ ID NO: 320 - protein, 13 aa, Homo sapiens
SVEGGSFGANVLT

SEQ ID NO: 321 - protein, 13 aa, Homo sapiens
ASSPGRILTDTQY

SEQ ID NO: 322 - protein, 10 aa, Homo sapiens
ASSLADYEQY

SEQ ID NO: 323 - protein, 11 aa, Homo sapiens
ASSGGLNQPQH

SEQ ID NO: 324 - protein, 12 aa, Homo sapiens
ASKVQGSEDTQY

SEQ ID NO: 325 - protein, 13 aa, Homo sapiens
ASSPPGGFGNEQF

SEQ ID NO: 326 - protein, 14 aa, Homo sapiens
ASSGAGQGSSYEQY

SEQ ID NO: 327 - protein, 10 aa, Homo sapiens
ASSLASCEQY

SEQ ID NO: 328 - protein, 10 aa, Homo sapiens
ASSLAGYRQY

SEQ ID NO: 329 - protein, 10 aa, Homo sapiens
ASSLAGYKQY

SEQ ID NO: 330 - protein, 12 aa, Homo sapiens
ASTSWASPTMSS

SEQ ID NO: 331 - protein, 13 aa, Homo sapiens
ASTSRGVSYNEQF

SEQ ID NO: 332 - protein, 11 aa, Homo sapiens
ASSFGQGKHSF

SEQ ID NO: 333 - protein, 10 aa, Homo sapiens
ASSGQGKHSF

SEQ ID NO: 334 - protein, 10 aa, Homo sapiens
ASSLVGHEQY

SEQ ID NO: 335 - protein, 10 aa, Homo sapiens
ASSLAGHGQY

SEQ ID NO: 336 - protein, 10 aa, Homo sapiens
ASNSAGYEQY

SEQ ID NO: 337 - protein, 10 aa, Homo sapiens
ASGSAGYEQY

SEQ ID NO: 338 - protein, 13 aa, Homo sapiens
ASTSWGISYNEQF

SEQ ID NO: 339 - protein, 13 aa, Homo sapiens
ASTSWGASYNEQF

SEQ ID NO: 340 - protein, 12 aa, Homo sapiens
ASTSGVSYNEQF

SEQ ID NO: 341 - protein, 13 aa, Homo sapiens
AGTSWGVSYNEQF

SEQ ID NO: 342 - protein, 11 aa, Homo sapiens
ASSVGQGKHSF

SEQ ID NO: 343 - protein, 11 aa, Homo sapiens
ASSMGQGKHSF

SEQ ID NO: 344 - protein, 11 aa, Homo sapiens
ASSLCQGKHSF

SEQ ID NO: 345 - protein, 12 aa, Homo sapiens
ASRGLAGFNEQF

SEQ ID NO: 346 - protein, 15 aa, Homo sapiens
ASSLMRVGFRTDTQY

SEQ ID NO: 347 - protein, 13 aa, Homo sapiens
ASSQDELAGRTQY

SEQ ID NO: 348 - protein, 13 aa, Homo sapiens
ASTNSLTSTDTQY

SEQ ID NO: 349 - protein, 13 aa, Homo sapiens
ASSPPEGLGNEQF

SEQ ID NO: 350 - protein, 11 aa, Homo sapiens
AISRADQETQY

SEQ ID NO: 351 - protein, 12 aa, Homo sapiens
SARDRGATGELF

SEQ ID NO: 352 - protein, 13 aa, Homo sapiens
SVEGGSAGANVLT

SEQ ID NO: 353 - protein, 10 aa, Homo sapiens
SARQGRTEAF

SEQ ID NO: 354 - protein, 13 aa, Homo sapiens
ASTSWGVPYNEQF

SEQ ID NO: 355 - protein, 10 aa, Homo sapiens
ASSLTSYEQY

SEQ ID NO: 356 - protein, 10 aa, Homo sapiens
ASSLTGCEQY

SEQ ID NO: 357 - protein, 11 aa, Homo sapiens
ASSLGQRKHSF

SEQ ID NO: 358 - protein, 9 aa, Homo sapiens
ASSLAGYKY

SEQ ID NO: 359 - protein, 10 aa, Homo sapiens
ASGMAGYEQY

SEQ ID NO: 360 - protein, 12 aa, Homo sapiens
ASSWARGNTEAF

SEQ ID NO: 361 - protein, 11 aa, Homo sapiens
ASSLGQETLKL

SEQ ID NO: 362 - protein, 9 aa, Homo sapiens
ASTLYEKLF

SEQ ID NO: 363 - protein, 10 aa, Homo sapiens
ASDLAGYEQY

SEQ ID NO: 364 - protein, 12 aa, Homo sapiens
ASVGTGNVDEQY

SEQ ID NO: 365 - protein, 13 aa, Homo sapiens
ASSPPEGSGNEQF

SEQ ID NO: 366 - protein, 13 aa, Homo sapiens
ASSPPEGFSNEQF

SEQ ID NO: 367 - protein, 13 aa, Homo sapiens
SANLARSSYNEQF

SEQ ID NO: 368 - protein, 13 aa, Homo sapiens
SALDLAGSQETQY

SEQ ID NO: 369 - protein, 12 aa, Homo sapiens
AVELFAAGNKLT

SEQ ID NO: 370 - protein, 12 aa, Homo sapiens
AGAVTGQLQQIL

SEQ ID NO: 371 - protein, 12 aa, HoJo virus
AVGLFAAGNKLT

SEQ ID NO: 372 - protein, 12 aa, Homo sapiens
AVELLAAGNKLT

SEQ ID NO: 373 - protein, 12 aa, Homo sapiens
AVELSAAGNKLT

SEQ ID NO: 374 - protein, 12 aa, Homo sapiens
AVKLFAAGNKLT

SEQ ID NO: 375 - protein, 12 aa, Homo sapiens
AVELFTAGNKLT

SEQ ID NO: 376 - protein, 12 aa, Homo sapiens
AVELFATGNKLT

SEQ ID NO: 377 - protein, 12 aa, Homo sapiens
AVELFVAGNKLT

SEQ ID NO: 378 - protein, 11 aa, Homo sapiens
AVELFAAGNKL

SEQ ID NO: 379 - protein, 12 aa, Homo sapiens
AVVLFAAGNKLT

SEQ ID NO: 380 - protein, 10 aa, Homo sapiens
AVSYLLQATS

SEQ ID NO: 381 - protein, 11 aa, Homo sapiens
AVLFAAGNKLT

SEQ ID NO: 382 - protein, 12 aa, Homo sapiens
AVELFASGNKLT

SEQ ID NO: 383 - protein, 10 aa, Homo sapiens
AVELFAAATS

SEQ ID NO: 384 - protein, 12 aa, Homo sapiens
AVDLFAAGNKLT

SEQ ID NO: 385 - protein, 12 aa, Homo sapiens
AVELFDAGNKLT

SEQ ID NO: 386 - protein, 11 aa, Homo sapiens
AASEGNYNVLY

SEQ ID NO: 387 - protein, 12 aa, Homo sapiens
AVEVFAAGNKLT

SEQ ID NO: 388 - protein, 12 aa, Homo sapiens
AVELFSAGNKLT

SEQ ID NO: 389 - protein, 12 aa, Homo sapiens
AVALFAAGNKLT

SEQ ID NO: 390 - protein, 12 aa, Homo sapiens
AVEIFAAGNKLT

SEQ ID NO: 391 - protein, 12 aa, Homo sapiens
AVELFPAGNKLT

SEQ ID NO: 392 - protein, 12 aa, Homo sapiens
AVELFGAGNKLT

SEQ ID NO: 393 - protein, 10 aa, Homo sapiens
AVELFAEATS

SEQ ID NO: 394 - protein, 13 aa, Homo sapiens
ASSLISGSSYEQY

SEQ ID NO: 395 - protein, 12 aa, Homo sapiens
ASSIEGQKTLKL

SEQ ID NO: 396 - protein, 13 aa, Homo sapiens
ASSLISGSPYEQY

SEQ ID NO: 397 - protein, 13 aa, Homo sapiens
ASGLISGSSYEQY

SEQ ID NO: 398 - protein, 13 aa, Homo sapiens
ASSLIGGSSYEQY

SEQ ID NO: 399 - protein, 13 aa, Homo sapiens
ASSLVSGSSYEQY

SEQ ID NO: 400 - protein, 13 aa, Homo sapiens
ASSPISGSSYEQY

SEQ ID NO: 401 - protein, 13 aa, Homo sapiens
ASSLISGGSYEQY

SEQ ID NO: 402 - protein, 13 aa, Homo sapiens
ASNLISGSSYEQY

SEQ ID NO: 403 - protein, 13 aa, Homo sapiens
ASSLISGSFYEQY

SEQ ID NO: 404 - protein, 13 aa, Homo sapiens
ASSLISGSTYEQY

SEQ ID NO: 405 - protein, 12 aa, Homo sapiens
ASSIEGQKALKL

SEQ ID NO: 406 - protein, 13 aa, Homo sapiens
ASRLISGSSYEQY

SEQ ID NO: 407 - protein, 13 aa, Homo sapiens
ASSIEGQKWTLKL

SEQ ID NO: 408 - protein, 10 aa, Homo sapiens
AAVVVAPTSS

SEQ ID NO: 409 - protein, 12 aa, Homo sapiens
ASSIGGQKTLKL

SEQ ID NO: 410 - protein, 12 aa, Homo sapiens
ASSIEGQRTLKL

SEQ ID NO: 411 - protein, 12 aa, Homo sapiens
ASSIEGRKTLKL

SEQ ID NO: 412 - protein, 12 aa, Homo sapiens
ASSTEGQKTLKL

SEQ ID NO: 413 - protein, 12 aa, Homo sapiens
ASSLISVAPTSS

SEQ ID NO: 414 - protein, 12 aa, Homo sapiens
ASSIEGQETLKL

SEQ ID NO: 415 - protein, 13 aa, Homo sapiens
ARSLISGSSYEQY

SEQ ID NO: 416 - protein, 12 aa, Homo sapiens
ASSMEGQKTLKL

SEQ ID NO: 417 - protein, 12 aa, Homo sapiens
ASSIEEQKTLKL

SEQ ID NO: 418 - protein, 13 aa, Homo sapiens
ASSIEGQKRTLKL

SEQ ID NO: 419 - protein, 13 aa, Homo sapiens
ASSLISGSAYEQY

SEQ ID NO: 420 - protein, 13 aa, Homo sapiens
ASCLISGSSYEQY

SEQ ID NO: 421 - protein, 13 aa, Homo sapiens
ASSLISGRSYEQY

SEQ ID NO: 422 - protein, 13 aa, Homo sapiens
ASSLISGSYYEQY

SEQ ID NO: 423 - protein, 13 aa, Homo sapiens
ASSIEGQKCTLKL

SEQ ID NO: 424 - protein, 12 aa, Homo sapiens
ASSLSGSSYEQY

SEQ ID NO: 425 - protein, 12 aa, Homo sapiens
ASSLISGSSTSS

SEQ ID NO: 426 - protein, 13 aa, Homo sapiens
ASSLISGSSREQY

SEQ ID NO: 427 - protein, 13 aa, Homo sapiens
ASILISGSSYEQY

SEQ ID NO: 428 - protein, 12 aa, Homo sapiens
ASSKEGQKTLKL

SEQ ID NO: 429 - protein, 13 aa, Homo sapiens
ASTLISGSSYEQY

SEQ ID NO: 430 - protein, 13 aa, Homo sapiens
ASSPISGSPYEQY

SEQ ID NO: 431 - protein, 13 aa, Homo sapiens
ASSLVSGNSYEQY

SEQ ID NO: 432 - protein, 13 aa, Homo sapiens
ASSLISGSCYEQY

SEQ ID NO: 433 - protein, 13 aa, Homo sapiens
ASGPISGSSYEQY

SEQ ID NO: 434 - protein, 13 aa, Homo sapiens
ASGLISGGSYEQY

SEQ ID NO: 435 - protein, 13 aa, Homo sapiens
ASGLIGGSSYEQY

SEQ ID NO: 436 - protein, 11 aa, Homo sapiens
ASSIEGQKHSF

SEQ ID NO: 437 - protein, 12 aa, Homo sapiens
ASSREGQKTLKL

SEQ ID NO: 438 - protein, 12 aa, Homo sapiens
ASSIKGQKTLKL

SEQ ID NO: 439 - protein, 13 aa, Homo sapiens
ASSIEGQKGTLKL

SEQ ID NO: 440 - protein, 11 aa, Homo sapiens
APPPGSARQLT

SEQ ID NO: 441 - protein, 9 aa, Homo sapiens
AVMNAGKST

SEQ ID NO: 442 - protein, 11 aa, Homo sapiens
ALPSGSARQLT

SEQ ID NO: 443 - protein, 11 aa, Homo sapiens
APPSSSARQLT

SEQ ID NO: 444 - protein, 11 aa, Homo sapiens
TPPSGSARQLT

SEQ ID NO: 445 - protein, 11 aa, Homo sapiens
VPPSGSARQLT

SEQ ID NO: 446 - protein, 11 aa, Homo sapiens
APSSGSARQLT

SEQ ID NO: 447 - protein, 11 aa, Homo sapiens
APPFGSARQLT

SEQ ID NO: 448 - protein, 11 aa, Homo sapiens
APPTGSARQLT

SEQ ID NO: 449 - protein, 11 aa, Homo sapiens
AENEDYGQNFV

SEQ ID NO: 450 - protein, 10 aa, Homo sapiens
APPSGSARQL

SEQ ID NO: 451 - protein, 52 aa, Homo sapiens
APPSGSARQLTFGSGTQLTVLPEHIKKRGEVTKGSLLGIKHCDTHGRRKQTH

SEQ ID NO: 452 - protein, 11 aa, Homo sapiens
AQPSGSARQLT

SEQ ID NO: 453 - protein, 11 aa, Homo sapiens
APPSCSARQLT

SEQ ID NO: 454 - protein, 11 aa, Homo sapiens
APPAGSARQLT

SEQ ID NO: 455 - protein, 11 aa, Homo sapiens
APPYGSARQLT

SEQ ID NO: 456 - protein, 11 aa, Homo sapiens
DPPSGSARQLT

SEQ ID NO: 457 - protein, 11 aa, Homo sapiens
GPPSGSARQLT

SEQ ID NO: 458 - protein, 12 aa, Homo sapiens
AVNIGGSQGNLI

SEQ ID NO: 459 - protein, 11 aa, Homo sapiens
SPPSGSARQLT

SEQ ID NO: 460 - protein, 11 aa, Homo sapiens
APPSRSARQLT

SEQ ID NO: 461 - protein, 9 aa, Homo sapiens
ARPLVLQGN

SEQ ID NO: 462 - protein, 11 aa, Homo sapiens
PPPSGSARQLT

SEQ ID NO: 463 - protein, 11 aa, Homo sapiens
APPSDSARQLT

SEQ ID NO: 464 - protein, 10 aa, Homo sapiens
AVYSGYSTLT

SEQ ID NO: 465 - protein, 13 aa, Homo sapiens
AVNARDSGTYKYI

SEQ ID NO: 466 - protein, 12 aa, Homo sapiens
AAPGECWQQPAD

SEQ ID NO: 467 - protein, 14 aa, Homo sapiens
ARTGYSGGGADGLT

SEQ ID NO: 468 - protein, 11 aa, Homo sapiens
AYDQGGSEKLV

SEQ ID NO: 469 - protein, 12 aa, Homo sapiens
AMSFRGGYQKVT

SEQ ID NO: 470 - protein, 11 aa, Homo sapiens
APPSGPARQLT

SEQ ID NO: 471 - protein, 11 aa, Homo sapiens
APPSDPARQLT

SEQ ID NO: 472 - protein, 11 aa, Homo sapiens
APPCGSARQLT

SEQ ID NO: 473 - protein, 11 aa, Homo sapiens
AVRPIEHRPVL

SEQ ID NO: 474 - protein, 11 aa, Homo sapiens
ALRSGGYQKVT

SEQ ID NO: 475 - protein, 9 aa, Homo sapiens
AVGAGGKLI

SEQ ID NO: 476 - protein, 9 aa, Homo sapiens
AVTNAGKST

SEQ ID NO: 477 - protein, 9 aa, Homo sapiens
AVMSAGKST

SEQ ID NO: 478 - protein, 9 aa, Homo sapiens
AVMDAGKST

SEQ ID NO: 479 - protein, 7 aa, Homo sapiens
PLVLQGN

SEQ ID NO: 480 - protein, 52 aa, Homo sapiens
APPSGSARQLTFGSGTQLTVLPEHIKKRGEVTKGSLLGIKHCETHGRRKQTH

SEQ ID NO: 481 - protein, 11 aa, Homo sapiens
APPPDSARQLT

SEQ ID NO: 482 - protein, 12 aa, Homo sapiens
AMSLDNYGQNFV

SEQ ID NO: 483 - protein, 11 aa, Homo sapiens
AFILQGAQKLV

SEQ ID NO: 484 - protein, 10 aa, Homo sapiens
ALELSGYALN

SEQ ID NO: 485 - protein, 10 aa, Homo sapiens
IWLRADLKSW

SEQ ID NO: 486 - protein, 11 aa, Homo sapiens
AYGSSNTGKLI

SEQ ID NO: 487 - protein, 10 aa, Homo sapiens
AVSARRQNFV

SEQ ID NO: 488 - protein, 9 aa, Homo sapiens
AVRNAGKST

SEQ ID NO: 489 - protein, 9 aa, Homo sapiens
AMMNAGKST

SEQ ID NO: 490 - protein, 11 aa, Homo sapiens
VPSSGSARQLT

SEQ ID NO: 491 - protein, 11 aa, Homo sapiens
TPPFGSARQLT

SEQ ID NO: 492 - protein, 11 aa, Homo sapiens
TPLSGSARQLT

SEQ ID NO: 493 - protein, 10 aa, Homo sapiens
SALWFCKATD

SEQ ID NO: 494 - protein, 11 aa, Homo sapiens
ATPPGSARQLT

SEQ ID NO: 495 - protein, 11 aa, Homo sapiens
ASPPGSARQLT

SEQ ID NO: 496 - protein, 12 aa, Homo sapiens
APSSAGNNRKLI

SEQ ID NO: 497 - protein, 9 aa, Homo sapiens
APPSVLQGN

SEQ ID NO: 498 - protein, 52 aa, Homo sapiens
APPSGSARQLTFGSGTQLAVLPEHIKKRGEVTKGSLLGIKHCDTHGRRKQTH

SEQ ID NO: 499 - protein, 10 aa, Homo sapiens
APPSGSAGNW

SEQ ID NO: 500 - protein, 11 aa, Homo sapiens
APPSGLARQLT

SEQ ID NO: 501 - protein, 10 aa, Homo sapiens
APPPGSARQL

SEQ ID NO: 502 - protein, 11 aa, Homo sapiens
APPLGSARQLT

SEQ ID NO: 503 - protein, 11 aa, Homo sapiens
ALPPGSARQLT

SEQ ID NO: 504 - protein, 10 aa, Homo sapiens
ALDLTGNQFY

SEQ ID NO: 505 - protein, 13 aa, Homo sapiens
AASLSNFGNEKLT

SEQ ID NO: 506 - protein, 14 aa, Homo sapiens
AASFSDQTGANNLF

SEQ ID NO: 507 - protein, 13 aa, Homo sapiens
ASSPPEGFGDEQF

SEQ ID NO: 508 - protein, 13 aa, Homo sapiens
ASSPPEGFGSEQF

SEQ ID NO: 509 - protein, 13 aa, Homo sapiens
ASSPPEGFDNEQF

SEQ ID NO: 510 - protein, 13 aa, Homo sapiens
ASSLPEGFGNEQF

SEQ ID NO: 511 - protein, 13 aa, Homo sapiens
ASSPLEGFGNEQF

SEQ ID NO: 512 - protein, 13 aa, Homo sapiens
ASSPSEGFGNEQF

SEQ ID NO: 513 - protein, 12 aa, Homo sapiens
ASSPPEGLAMSS

SEQ ID NO: 514 - protein, 12 aa, Homo sapiens
ASSHLRVLAMSS

SEQ ID NO: 515 - protein, 12 aa, Homo sapiens
ASSPPGLGYEQY

SEQ ID NO: 516 - protein, 12 aa, Homo sapiens
ASSPRGGSYEQY

SEQ ID NO: 517 - protein, 13 aa, Homo sapiens
ASSPPEGFGKEQF

SEQ ID NO: 518 - protein, 12 aa, Homo sapiens
AIRSTASTDTQY

SEQ ID NO: 519 - protein, 13 aa, Homo sapiens
ASSPHEGFGNEQF

SEQ ID NO: 520 - protein, 12 aa, Homo sapiens
AAHHLRVLAMSS

SEQ ID NO: 521 - protein, 13 aa, Homo sapiens
ASSPPEGFGYEQF

SEQ ID NO: 522 - protein, 13 aa, Homo sapiens
ASSQPEGFGNEQF

SEQ ID NO: 523 - protein, 11 aa, Homo sapiens
ASRGTSVQQAV

SEQ ID NO: 524 - protein, 13 aa, Homo sapiens
ASTPSGPSTDTQY

SEQ ID NO: 525 - protein, 13 aa, Homo sapiens
ASSPPEGFGIEQF

SEQ ID NO: 526 - protein, 15 aa, Homo sapiens
ASSYSGAGGPWDTQY

SEQ ID NO: 527 - protein, 13 aa, Homo sapiens
ASSPPEGFGTEQF

SEQ ID NO: 528 - protein, 13 aa, Homo sapiens
ASSVEGTGTSIQY

SEQ ID NO: 529 - protein, 13 aa, Homo sapiens
ASSPPEGFGHEQF

SEQ ID NO: 530 - protein, 13 aa, Homo sapiens
ASSPTEGFGNEQF

SEQ ID NO: 531 - protein, 10 aa, Homo sapiens
ASSLWGTEAF

SEQ ID NO: 532 - protein, 13 aa, Homo sapiens
ASSRPEGFGNEQF

SEQ ID NO: 533 - protein, 13 aa, Homo sapiens
ASSPREGFGNEQF

SEQ ID NO: 534 - protein, 12 aa, Homo sapiens
ASSLDRLYTEAF

SEQ ID NO: 535 - protein, 12 aa, Homo sapiens
ASSFGTGGNTQY

SEQ ID NO: 536 - protein, 13 aa, Homo sapiens
ASSPAEGFGNEQF

SEQ ID NO: 537 - protein, 11 aa, Homo sapiens
ASSLYFGQPQH

SEQ ID NO: 538 - protein, 10 aa, Homo sapiens
ASRTEAREQY

SEQ ID NO: 539 - protein, 11 aa, Homo sapiens
ASSQPGQYGYT

SEQ ID NO: 540 - protein, 13 aa, Homo sapiens
ASSPPEGFGDGQF

SEQ ID NO: 541 - protein, 10 aa, Homo sapiens
ASRLGHQPQH

SEQ ID NO: 542 - protein, 13 aa, Homo sapiens
ASSPPKGFGNEQF

SEQ ID NO: 543 - protein, 13 aa, Homo sapiens
ASSPPEGFGNRQF

SEQ ID NO: 544 - protein, 13 aa, Homo sapiens
ASPPPEGFGNEQF

SEQ ID NO: 545 - protein, 15 aa, Homo sapiens
SVGVTGGTITPHEQY

SEQ ID NO: 546 - protein, 17 aa, Homo sapiens
ASSDRDRDGRARGGEQF

SEQ ID NO: 547 - protein, 13 aa, Homo sapiens
ASSPPKGLGNEQF

SEQ ID NO: 548 - protein, 13 aa, Homo sapiens
ASSPPESLGNEQF

SEQ ID NO: 549 - protein, 13 aa, Homo sapiens
ASSPPEGFGNKQF

SEQ ID NO: 550 - protein, 12 aa, Homo sapiens
AWRPGYMNTEAF

SEQ ID NO: 551 - protein, 15 aa, Homo sapiens
AISEWASGRPSYEQY

SEQ ID NO: 552 - protein, 13 aa, Homo sapiens
ASSALAGDTYEQY

SEQ ID NO: 553 - protein, 12 aa, Homo sapiens
ASSPRRGSYEQY

SEQ ID NO: 554 - protein, 11 aa, Homo sapiens
ASSPRGAPTSS

SEQ ID NO: 555 - protein, 13 aa, Homo sapiens
ASSYSPGNHQPQH

SEQ ID NO: 556 - protein, 13 aa, Homo sapiens
ASSPSEGFDNEQF

SEQ ID NO: 557 - protein, 13 aa, Homo sapiens
ASSPPEGLSNEQF

SEQ ID NO: 558 - protein, 13 aa, Homo sapiens
ASSPPEGFSSEQF

SEQ ID NO: 559 - protein, 12 aa, Homo sapiens
ASSPPEGFGNEF

SEQ ID NO: 560 - protein, 13 aa, Homo sapiens
ASSPPEDFGNEQF

SEQ ID NO: 561 - protein, 12 aa, Homo sapiens
AIRSTASADTQY

SEQ ID NO: 562 - protein, 12 aa, Homo sapiens
ATTRTSGSNEQF

SEQ ID NO: 563 - protein, 12 aa, Homo sapiens
ASSPRGGLLRAV

SEQ ID NO: 564 - protein, 13 aa, Homo sapiens
ASSYGLAHSYEQY

SEQ ID NO: 565 - protein, 10 aa, Homo sapiens
ATLQGPNEQF

SEQ ID NO: 566 - protein, 14 aa, Homo sapiens
ASSSSVLRARTEAF

SEQ ID NO: 567 - protein, 14 aa, Homo sapiens
ASSIFLGDNTGELF

SEQ ID NO: 568 - protein, 13 aa, Homo sapiens
ASLPPEGFGNEQF

SEQ ID NO: 569 - protein, 13 aa, Homo sapiens
ASSPSEDFGNEQF

SEQ ID NO: 570 - protein, 13 aa, Homo sapiens
ASSPPKGSGNEQF

SEQ ID NO: 571 - protein, 13 aa, Homo sapiens
ASSPPGGFDNEQF

SEQ ID NO: 572 - protein, 13 aa, Homo sapiens
ASSPPEGFNNEQF

SEQ ID NO: 573 - protein, 13 aa, Homo sapiens
ASSPPEGFGYGQF

SEQ ID NO: 574 - protein, 13 aa, Homo sapiens
ASSPPEGFGNGQF

SEQ ID NO: 575 - protein, 13 aa, Homo sapiens
ASSPPEDSGNEQF

SEQ ID NO: 576 - protein, 13 aa, Homo sapiens
ASSPPEDFDNEQF

SEQ ID NO: 577 - protein, 13 aa, Homo sapiens
ASSPLKGFGNEQF

SEQ ID NO: 578 - protein, 13 aa, Homo sapiens
ASSPLGGFGNEQF

SEQ ID NO: 579 - protein, 13 aa, Homo sapiens
ASSPLEGFSNEQF

SEQ ID NO: 580 - protein, 13 aa, Homo sapiens
ASSPLEDFGNEQF

SEQ ID NO: 581 - protein, 13 aa, Homo sapiens
ASSLPEGFDNEQF

SEQ ID NO: 582 - protein, 13 aa, Homo sapiens
ASPLPEGFGNEQF

SEQ ID NO: 583 - protein, 10 aa, Homo sapiens
SVWTDSDTQY

SEQ ID NO: 584 - protein, 11 aa, Homo sapiens
ASSGPSGQPQH

SEQ ID NO: 585 - protein, 17 aa, Homo sapiens
ASLSSPPRDPWRLIHPS

SEQ ID NO: 586 - protein, 11 aa, Homo sapiens
AASGANTNKVV

SEQ ID NO: 587 - protein, 10 aa, Homo sapiens
APYAGRRALT

SEQ ID NO: 588 - protein, 10 aa, Homo sapiens
APCTGRRALT

SEQ ID NO: 589 - protein, 10 aa, Homo sapiens
TPYTGRRALT

SEQ ID NO: 590 - protein, 10 aa, Homo sapiens
VPYTGRRALT

SEQ ID NO: 591 - protein, 10 aa, Homo sapiens
APYMGRRALT

SEQ ID NO: 592 - protein, 9 aa, Homo sapiens
APYTGRRAL

SEQ ID NO: 593 - protein, 12 aa, Homo sapiens
AGQDQDSGYALN

SEQ ID NO: 594 - protein, 9 aa, Homo sapiens
APTRAGEHL

SEQ ID NO: 595 - protein, 10 aa, Homo sapiens
AANSNDYKLS

SEQ ID NO: 596 - protein, 10 aa, Homo sapiens
APYPGRRALT

SEQ ID NO: 597 - protein, 10 aa, Homo sapiens
APYKGRRALT

SEQ ID NO: 598 - protein, 10 aa, Homo sapiens
APYSGRRALT

SEQ ID NO: 599 - protein, 13 aa, Homo sapiens
ALLDSGGGADGLT

SEQ ID NO: 600 - protein, 10 aa, Homo sapiens
GPYTGRRALT

SEQ ID NO: 601 - protein, 11 aa, Homo sapiens
AASGANTNKAV

SEQ ID NO: 602 - protein, 10 aa, Homo sapiens
APSTGRRALT

SEQ ID NO: 603 - protein, 9 aa, Homo sapiens
APTGRRALT

SEQ ID NO: 604 - protein, 14 aa, Homo sapiens
AVSAITQGGSEKLV

SEQ ID NO: 605 - protein, 10 aa, Homo sapiens
SPYTGRRALT

SEQ ID NO: 606 - protein, 10 aa, Homo sapiens
PPYTGRRALT

SEQ ID NO: 607 - protein, 10 aa, Homo sapiens
EPYTGRRALT

SEQ ID NO: 608 - protein, 9 aa, Homo sapiens
APYTAGEHL

SEQ ID NO: 609 - protein, 9 aa, Homo sapiens
AGPGGQLID

SEQ ID NO: 610 - protein, 9 aa, Homo sapiens
APSRAGEHL

SEQ ID NO: 611 - protein, 10 aa, Homo sapiens
AERYNTDKLI

SEQ ID NO: 612 - protein, 11 aa, Homo sapiens
AASGANTNKFV

SEQ ID NO: 613 - protein, 11 aa, Homo sapiens
ASSLGQGNEAF

SEQ ID NO: 614 - protein, 11 aa, Homo sapiens
SAREGSGNEQF

SEQ ID NO: 615 - protein, 11 aa, Homo sapiens
ASSLGQGDEAF

SEQ ID NO: 616 - protein, 11 aa, Homo sapiens
ASSLGRGNEAF

SEQ ID NO: 617 - protein, 11 aa, Homo sapiens
ASSLGQGSEAF

SEQ ID NO: 618 - protein, 11 aa, Homo sapiens
ASSLGQGNGAF

SEQ ID NO: 619 - protein, 11 aa, Homo sapiens
ASSSGQGNEAF

SEQ ID NO: 620 - protein, 11 aa, Homo sapiens
ASSLYLGQPQH

SEQ ID NO: 621 - protein, 11 aa, Homo sapiens
SASSGSTDTQY

SEQ ID NO: 622 - protein, 11 aa, Homo sapiens
ASSLYFGRPQH

SEQ ID NO: 623 - protein, 11 aa, Homo sapiens
ASSLYSGQPQH

SEQ ID NO: 624 - protein, 11 aa, Homo sapiens
ASGLYFGQPQH

SEQ ID NO: 625 - protein, 10 aa, Homo sapiens
ASSLYFGQPS

SEQ ID NO: 626 - protein, 11 aa, Homo sapiens
ASSLGQGNKAF

SEQ ID NO: 627 - protein, 11 aa, Homo sapiens
ASSLCFGQPQH

SEQ ID NO: 628 - protein, 12 aa, Homo sapiens
SVAGTYSYNEQF

SEQ ID NO: 629 - protein, 10 aa, Homo sapiens
SARDQIREQF

SEQ ID NO: 630 - protein, 15 aa, Homo sapiens
ASSDDPRESGANVLT

SEQ ID NO: 631 - protein, 11 aa, Homo sapiens
ASNLGQGNEAF

SEQ ID NO: 632 - protein, 10 aa, Homo sapiens
ASSLDRGMKL

SEQ ID NO: 633 - protein, 12 aa, Homo sapiens
SVEGTGGLNEQF

SEQ ID NO: 634 - protein, 10 aa, Homo sapiens
ASSLGQGMKL

SEQ ID NO: 635 - protein, 14 aa, Homo sapiens
ASSLGWRGNSYEQY

SEQ ID NO: 636 - protein, 10 aa, Homo sapiens
ASSGQGNEAF

SEQ ID NO: 637 - protein, 11 aa, Homo sapiens
ASNLYFGQPQH

SEQ ID NO: 638 - protein, 10 aa, Homo sapiens
ASSLYFGQPQ

SEQ ID NO: 639 - protein, 10 aa, Homo sapiens
ASSLYFGPQH

SEQ ID NO: 640 - protein, 11 aa, Homo sapiens
ASSLVSGGNEQ

SEQ ID NO: 641 - protein, 11 aa, Homo sapiens
ASSLYFGQSQH

SEQ ID NO: 642 - protein, 8 aa, Homo sapiens
AWEEGEAF

SEQ ID NO: 643 - protein, 10 aa, Homo sapiens
ASSLGQGNEA

SEQ ID NO: 644 - protein, 11 aa, Homo sapiens
ASSLGQGNVAF

SEQ ID NO: 645 - protein, 11 aa, Homo sapiens
ASSLGQGKEAF

SEQ ID NO: 646 - protein, 9 aa, Homo sapiens
SVDLGWEQY

SEQ ID NO: 647 - protein, 12 aa, Homo sapiens
ATSDLTGGNEQF

SEQ ID NO: 648 - protein, 12 aa, Homo sapiens
SVELAGEADTQY

SEQ ID NO: 649 - protein, 10 aa, Homo sapiens
AAVYTLGSPS

SEQ ID NO: 650 - protein, 11 aa, Homo sapiens
ASSMGQGNEAF

SEQ ID NO: 651 - protein, 10 aa, Homo sapiens
ASSYTLGSPS

SEQ ID NO: 652 - protein, 11 aa, Homo sapiens
ASSLGQGNDAF

SEQ ID NO: 653 - protein, 10 aa, Homo sapiens
ASSLYFGSPS

SEQ ID NO: 654 - protein, 15 aa, Homo sapiens
SASSGTSGRLYNEQF

SEQ ID NO: 655 - protein, 10 aa, Homo sapiens
AAAWDRGMKL

SEQ ID NO: 656 - protein, 11 aa, Homo sapiens
ASSWGQGNEAF

SEQ ID NO: 657 - protein, 11 aa, Homo sapiens
ASSVGQGNEAF

SEQ ID NO: 658 - protein, 11 aa, Homo sapiens
ASSLTLQETQY

SEQ ID NO: 659 - protein, 10 aa, Homo sapiens
SVGTSGYEQY

SEQ ID NO: 660 - protein, 12 aa, Homo sapiens
ASSYFAGPYEQY

SEQ ID NO: 661 - protein, 12 aa, Homo sapiens
AWSEGVGNQPQH

SEQ ID NO: 662 - protein, 10 aa, Homo sapiens
ASSYFGQPQH

SEQ ID NO: 663 - protein, 11 aa, Homo sapiens
ASSLGQGNAAF

SEQ ID NO: 664 - protein, 11 aa, Homo sapiens
ASILGQGNEAF

SEQ ID NO: 665 - protein, 19 aa, Homo sapiens
ASSLVGAQGLAGTNNYEQY

SEQ ID NO: 666 - protein, 11 aa, Homo sapiens
ASSLYFGPPQH

SEQ ID NO: 667 - protein, 11 aa, Homo sapiens
ASSLYFGLPQH

SEQ ID NO: 668 - protein, 11 aa, Homo sapiens
ASSLYFGHPQH

SEQ ID NO: 669 - protein, 11 aa, Homo sapiens
ASSIYFGQPQH

SEQ ID NO: 670 - protein, 11 aa, Homo sapiens
ASILYFGQPQH

SEQ ID NO: 671 - protein, 12 aa, Homo sapiens
ASSPGSAYNEQF

SEQ ID NO: 672 - protein, 14 aa, Homo sapiens
AISESLAGGYNEQF

SEQ ID NO: 673 - protein, 11 aa, Homo sapiens
ASRLGQGNEAF

SEQ ID NO: 674 - protein, 12 aa, Homo sapiens
ASSPTLGVDTQY

SEQ ID NO: 675 - protein, 12 aa, Homo sapiens
ASSVDGGEQPQH

SEQ ID NO: 676 - protein, 9 aa, Homo sapiens
ASSQYVEQY

SEQ ID NO: 677 - protein, 10 aa, Homo sapiens
ASSQGSDEQY

SEQ ID NO: 678 - protein, 15 aa, Homo sapiens
ASSLTGHREAYNEQF

SEQ ID NO: 679 - protein, 13 aa, Homo sapiens
ASSSTGGTSYGYT

SEQ ID NO: 680 - protein, 11 aa, Homo sapiens
ASSLGQGTEAF

SEQ ID NO: 681 - protein, 10 aa, Homo sapiens
ASSLGQGNAF

SEQ ID NO: 682 - protein, 8 aa, Homo sapiens
ASSLGQGN

SEQ ID NO: 683 - protein, 11 aa, Homo sapiens
ASSLGQGDGAF

SEQ ID NO: 684 - protein, 17 aa, Homo sapiens
ASSPRVPGQGTAGNTIY

SEQ ID NO: 685 - protein, 12 aa, Homo sapiens
ASSLSVGSGELF

SEQ ID NO: 686 - protein, 11 aa, Homo sapiens
ASSVYFGQPQH

SEQ ID NO: 687 - protein, 11 aa, Homo sapiens
ASSLYFGQTQH

SEQ ID NO: 688 - protein, 11 aa, Homo sapiens
ASSLYFGQAQH

SEQ ID NO: 689 - protein, 11 aa, Homo sapiens
ASSLYFGKPQH

SEQ ID NO: 690 - protein, 11 aa, Homo sapiens
ASRLYFGQPQH

SEQ ID NO: 691 - protein, 11 aa, Homo sapiens
ASSLYWVDTQY

SEQ ID NO: 692 - protein, 12 aa, Homo sapiens
ASSLAYTSTEAF

SEQ ID NO: 693 - protein, 12 aa, Homo sapiens
ASSLYQGPNEQF

SEQ ID NO: 694 - protein, 16 aa, Homo sapiens
ASSFRQWAGGGTDTQY

SEQ ID NO: 695 - protein, 13 aa, Homo sapiens
ASSLVQGTWYEQY

SEQ ID NO: 696 - protein, 13 aa, Homo sapiens
ASSLVGGAYNEQF

SEQ ID NO: 697 - protein, 10 aa, Homo sapiens
ASSWDRGMKL

SEQ ID NO: 698 - protein, 12 aa, Homo sapiens
ASSLVSGGNEQF

SEQ ID NO: 699 - protein, 11 aa, Homo sapiens
ASSLGQGNQAF

SEQ ID NO: 700 - protein, 10 aa, Homo sapiens
ASSLGQGNEV

SEQ ID NO: 701 - protein, 11 aa, Homo sapiens
ASSLGQGDKAF

SEQ ID NO: 702 - protein, 11 aa, Homo sapiens
ASGSGQGNEAF

SEQ ID NO: 703 - protein, 14 aa, Homo sapiens
ASSSLLASGLHTQY

SEQ ID NO: 704 - protein, 14 aa, Homo sapiens
ASSPGWRGNSYEQY

SEQ ID NO: 705 - protein, 10 aa, Homo sapiens
ASSLYSGQPS

SEQ ID NO: 706 - protein, 11 aa, Homo sapiens
ASSLYFGEPQH

SEQ ID NO: 707 - protein, 13 aa, Homo sapiens
ASSQLGQGAGEQY

SEQ ID NO: 708 - protein, 11 aa, Homo sapiens
SARGGSGNEQF

SEQ ID NO: 709 - protein, 11 aa, Homo sapiens
SAREGSGDEQF

(19) United States
(12) Patent Application Publication
(10) Pub. No.: US 2022/0175899 A1
(43) Pub. Date: Jun. 9, 2022

(54) CYTOTOXIC T LYMPHOCYTES SPECIFIC FOR MUTATED FORMS OF EPIDERMAL GROWTH FACTOR RECEPTOR FOR USE IN TREATING CANCER

(71) Applicant: The Board of Trustees of the Leland Stanford Junior University, Stanford, CA (US)

(72) Inventors: Hiromitsu NAKAUCHI, Woodside, CA (US); Tohinobu NISHIMURA, Stanford, CA (US)

(21) Appl. No.: 17/601,282

(22) PCT Filed: Apr. 7, 2020
(86) PCT No.: PCT/US2020/027020

§ 371 Date: Oct. 4, 2021

Related U.S. Application Data
(60) Provisional application No. 62/832,626, filed on Apr. 11, 2019.

(51) Int. Cl. A61K 39/00; C12N 5/0783; C12N 5/074; A61P 35/00

(52) U.S. Cl. CPC A61K 39/001104; C12N 5/0638; A61P 35/00; A61K 39/001154; C12N 5/0696

(57) ABSTRACT

Compositions, methods, and kits are provided for producing rejuvenated cytotoxic T cells (CTLs) specific for mutated neo-antigen epitopes expressed on cancerous cells, including epidermal growth factor receptor (EGFR) and KRAS neo-antigen epitopes. Antigenspecific CTLs are rejuvenated by reprogramming them into induced pluripotent stem cells (IPSCs) using Yamanaka factors and redifferentiating them back into CTLs while expanding their numbers. After redifferentiation, the IPSC-derived rejuvenated CTLs retain the antigen specificity of the original CTLs from which they were derived, but have the advantage of having longer telomeres and higher proliferative activity than the original CTLs. Pharmaceutical compositions comprising such IPSC-derived rejuvenated CTLs are useful for treating cancers expressing the mutated neo-antigen epitopes recognized by the original CTLs.

### BACKGROUND

Lung cancer is the leading cause of cancer-related deaths worldwide. Overexpression of epidermal growth factor receptor (EGFR) is observed in various malignancies, including lung cancer. EGFR activation induces many intracellular signaling pathways, such as those involving mitogen-activated protein kinase (MAPK), phosphatidylinositol 3-kinase (PI3K), and signal transducer and activator of transcription (STAT) family members (West et al. (2009) J. Thorac. Oncol. 4:s1029-s1039). EGFR activation triggers many intracellular signaling pathways, such as those involving mitogen-activated protein kinase (MAPK), phosphatidylinositol 3-kinase (PI3K), and signal transducer and activator of transcription (STAT), which cause tumor cell proliferation and promote tumor survival (West et al., supra; Jackman et al. (2009) Clin. Cancer Res. 15:5267-5273).

The EGFR pathway is an appropriate target for cancer therapy. Several agents that block this pathway have been developed and have become the standard of care, first-line treatment for lung cancer patients. EGFR-tyrosine kinase inhibitors (EGFR-TKIs), such as gefitinib and erlotinib, have demonstrated remarkable clinical activity against non-small cell lung cancer (NSCLC) that harbors activating EGFR mutations. However, patients frequently develop acquired resistance to EGFR-TKI therapy. Replacement of a threonine with a methionine at codon 790 of EGFR (EGFR T790M) is the most common acquired resistance mutation, and is present in ˜50% of cases of TKI resistance (Gao et al. (2016) Expert Rev. Anticancer Ther. 16(4):383-390, Noda et al. (2016) Expert Rev. Respir. Med. 10(5):547-556, van der Wekken et al. (2016) Crit. Rev. Oncol. Hematol. 100:107-116, Villadolid et al. (2015) Transl. Lung Cancer Res. 4(5):576-583, Black et al. (2015) R I Med J (2013) 98(10):25-28). Studies have found when T790M is introduced in vitro into sequences containing wild-type EGFR, an exon 19 deletion-EGFR, or L858R-EGFR, the resulting proteins are significantly more resistant to gefitinib in the constructs containing T790M. These specific mutation sequences are becoming the biosignatures of relapsed cancers (Berman et al. (2016) Transl. Lung Cancer Res. 2016 February; 5(1):138-142). New treatment strategies for NSCLC patients harboring the EGFR T790M mutation are needed.

Cancer Immunotherapy is a new class of cancer treatment, with unique characteristics that distinguish it from other kinds of cancer therapies. It exploits the fact that cancer cells often have subtly different antigens/molecules that the immune system can detect. Immunotherapy is used to provoke the immune system into attacking tumor cells with these antigens/molecules as targets. Major advantages of cancer immunotherapy over other therapeutic approaches are its high specificity and low toxicity against normal tissues. Adoptive T-cell immunotherapy is a form of cellular immunotherapy that involves transfusion of patients with functional T-cells. This is a potential therapeutic strategy for combating various types of cancer. Recent reports indicate that tumor-reactive T cells recognize various mutated epitopes suggesting that these are potentially immunogenic and, as tumor signatures, might serve as immunotherapeutic targets (Simon et al. (2015) Oncoimmunology 5 (1):e1104448, Hasegawa et al. (2015) PLoS One 10(12)). The effectiveness of adoptive immunotherapy, however, is often hampered by exhaustion of antigen-specific T cells during ex vivo expansion.

### SUMMARY

Compositions, methods, and kits are provided for producing rejuvenated cytotoxic T cells (CTLs) specific for mutated neo-antigen epitopes expressed on cancerous cells, including epidermal growth factor receptor (EGFR) and KRAS neo-antigen epitopes. Antigen-specific CTLs are rejuvenated by reprogramming them into induced pluripotent stem cells (IPSCs) using Yamanaka factors and redifferentiating them back into CTLs while expanding their numbers. After redifferentiation, the IPSC-derived rejuvenated CTLs retain the antigen specificity of the original CTLs from which they were derived, but have the advantage of having longer telomeres and higher proliferative activity than the original CTLs. Pharmaceutical compositions comprising such IPSC-derived rejuvenated CTLs are useful for treating cancers expressing the mutated neo-antigen epitopes recognized by the original CTLs.

In one aspect, a method of cellular immunotherapy is provided for treating a subject for a cancer expressing a mutated epidermal growth factor receptor (EGFR) or KRAS neo-antigen epitope. In certain embodiments, the method comprises: a) eliciting an antigen-specific cytotoxic T cell response by contacting CTLs with an antigen presenting cell presenting at its surface an immunogenic peptide comprising the mutated EGFR or KRAS neo-antigen epitope in a complex with major histocompatibility complex (MHC); b) isolating CTLs specific for the mutated EGFR or KRAS neo-antigen epitope; c) generating induced pluripotent stem cells (IPSC) from the CTLs specific for the mutated EGFR or KRAS neo-antigen epitope; d) differentiating the IPSCs into rejuvenated CTLs specific for the mutated EGFR or KRAS neo-antigen epitope; and e) administering a therapeutically effective amount of the rejuvenated CTLs specific for the mutated EGFR or KRAS neo-antigen epitope to the subject.

In certain embodiments, the neo-antigen epitope is a mutated EGFR neo-antigen comprising a mutation selected from the group consisting of a C797S mutation, a T790M mutation, an L858R mutation, and a deletion. In other embodiments, the neo-antigen epitope is a mutated KRAS neo-antigen comprising a mutation selected from the group consisting of a G12D mutation, a G12V mutation, and a G12C mutation.

In certain embodiments, the immunogenic peptide comprises an amino acid sequence selected from the group consisting of SEQ ID NOS:1-5, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity thereto, wherein the immunogenic peptide comprises the mutated EGFR or KRAS neo-antigen epitope.

In certain embodiments, the CTLs are contacted with the antigen presenting cell in vivo, ex vivo, or in vitro. The CTLs specific for the mutated EGFR or KRAS neo-antigen epitope may be isolated, for example, from tumor infiltrating lymphocytes or peripheral blood mononuclear cells.

In certain embodiments, the CTLs are provided in a biological sample. The biological sample may be collected from the subject to be treated or a donor. In certain embodiments, the biological sample is blood, a tumor biopsy, a cancerous tissue sample, or a malignant effusion fluid sample. In one embodiment, the cancerous tissue sample is a lung cancer tissue sample.

The CTLs may be autologous or allogeneic. In one embodiment, the CTLs are obtained from a donor that is human leukocyte antigen (HLA)-matched with the subject undergoing the cellular immunotherapy.

In certain embodiments, the antigen presenting cell is a dendritic cell or a macrophage. In other embodiments, the antigen presenting cell is a cancerous cell expressing the mutated epidermal growth factor receptor (EGFR) or KRAS neo-antigen epitope. In further embodiments, an artificial antigen presenting cell is used such as, but not limited to, an MHC multimer, a cellular artificial antigen presenting cell (e.g., fibroblasts or other cells genetically modified to express MHC and other CTL stimulating proteins, or an acellular antigen presenting cell (e.g., biocompatible particle such as a microparticle or nanoparticle carrying CTL stimulating proteins).

In certain embodiments, the rejuvenated CTLs express CD8.

In certain embodiments, the rejuvenated CTLs are expanded in vitro before being administered to the subject.

In certain embodiments, the therapeutically effective amount of the rejuvenated CTLs is provided in a composition. The composition may further comprise a pharmaceutically acceptable excipient. In some embodiments, the composition further comprises an adjuvant. In another embodiment, the composition further comprises an anti-cancer therapeutic agent.

In certain embodiments, the subject has lung cancer (e.g., non-small cell lung carcinoma).

In certain embodiments, multiple cycles of treatment are administered to the subject for a time period sufficient to effect at least a partial tumor response, or more preferably, a complete tumor response.

In certain embodiments, the cancer expresses a major histocompatibility complex (MHC) carrying a peptide comprising the mutated EGFR or KRAS neo-antigen epitope.

In certain embodiments, the method further comprises introducing a suicide gene into the rejuvenated CTLs. For example, a nucleic acid encoding an inducible caspase-9 may be introduced into the rejuvenated CTLs, wherein induction of expression of the caspase-9 results in apoptosis of the rejuvenated CTLs.

In another aspect, a method is provided for producing an induced pluripotent stem cell (IPSC)-derived rejuvenated cytotoxic T cell (CTL) specific for a mutated EGFR or KRAS neo-antigen epitope. In certain embodiments, the method comprises: a) obtaining a biological sample comprising cytotoxic T cells (CTLs); b) eliciting an antigen-specific cytotoxic T cell response by contacting cytotoxic T cells (CTLs) with an antigen presenting cell presenting at its surface an immunogenic peptide comprising a mutated EGFR or KRAS neo-antigen epitope in a complex with major histocompatibility complex; c) isolating a CTL specific for the mutated EGFR or KRAS neo-antigen epitope; d) generating an induced pluripotent stem cell (IPSO) from the CTL specific for the mutated EGFR or KRAS neo-antigen epitope; and e) differentiating the IPSO into a rejuvenated CTL specific for the mutated EGFR or KRAS neo-antigen epitope.

In another aspect, a composition is provided comprising an IPSO-derived rejuvenated CTL specific for a mutated EGFR or KRAS neo-antigen epitope described herein. The composition may further comprise a pharmaceutically acceptable excipient. In another embodiment, the composition further comprises an adjuvant. In a further embodiment, the composition further comprises one or more other anti-cancer therapeutic agents such as, but not limited to, chemotherapeutic agents, immunotherapeutic agents, or biologic agents.

In another aspect, kits are provided for practicing the methods described herein. In certain embodiments, a kit may comprise IPSO-derived rejuvenated CTLs specific for a mutated EGFR or KRAS neo-antigen epitope or reagents for preparing them. The kit may further comprise instructions for use, including instructions on methods of preparing the IPSC-derived rejuvenated CTLs and/or methods of using them in immunotherapy for treating cancer as described herein.

In another aspect, an immunogenic peptide is provided comprising an amino acid sequence selected from the group consisting of SEQ ID NOS:1-5, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity thereto, wherein the immunogenic peptide comprises the mutated EGFR or KRAS neo-antigen epitope.

In another aspect, a composition is provided comprising an immunogenic peptide described herein. The composition may further comprise a pharmaceutically acceptable excipient. In another embodiment, the composition further comprises an adjuvant. In a further embodiment, the composition further comprises one or more other anti-cancer therapeutic agents such as, but not limited to, chemotherapeutic agents, immunotherapeutic agents, or biologic agents.

In another aspect, an isolated antigen presenting cell is provided comprising a MHC carrying an immunogenic peptide described herein.

In another aspect, a method of cellular immunotherapy is provided for treating a subject for a cancer expressing a mutated EGFR or KRAS neo-antigen epitope. In certain embodiments, the method comprises: a) obtaining a biological sample comprising cytotoxic T cells (CTLs) from the subject; b) isolating CTLs specific for the mutated EGFR or KRAS neo-antigen epitope from the subject; c) generating induced pluripotent stem cells (IPSCs) from the CTLs specific for the mutated EGFR or KRAS neo-antigen epitope; d) differentiating the IPSCs into rejuvenated CTLs specific for the mutated EGFR or KRAS neo-antigen epitope; and e) administering a therapeutically effective amount of the rejuvenated CTLs specific for the mutated EGFR or KRAS neo-antigen epitope to the subject.

In another aspect, a method of cellular immunotherapy for treating cancer in a subject is provided, the method comprising eliciting an antigen-specific cytotoxic T cell (CTL) response by administering an immunogenic peptide comprising an amino acid sequence selected from the group consisting of SEQ ID NOS:1-5 to the subject.

In another aspect, a method of cellular immunotherapy for treating cancer in a subject, the method comprising: a) eliciting an antigen-specific cytotoxic T cell (CTL) response by administering an immunogenic peptide comprising a mutated epidermal growth factor receptor (EGFR) or KRAS neo-antigen epitope to the subject; b) obtaining a biological sample comprising CTLs from the subject; c) isolating CTLs specific for the mutated EGFR or KRAS neo-antigen epitope from the biological sample; d) generating induced pluripotent stem cells (IPSCs) from the CTLs specific for the mutated EGFR or KRAS neo-antigen epitope; e) differentiating the IPSCs into rejuvenated CTLs specific for the mutated EGFR or KRAS neo-antigen epitope; and f) administering a therapeutically effective amount of the rejuvenated CTLs specific for the mutated EGFR or KRAS neo-antigen epitope to the subject.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 shows a schematic of the strategy for rejuvenation of antigen-specific T cells using iPSC technology. T-iPS cells were generated from antigen-specific T cells, expanded in vitro, and re-differentiated into antigen-specific T cells.

FIGS. 2A and 2B show the efficacy of EBV-CTL. FIG. 2A shows light emission monitored as indicator of tumor growth in mice. Around 5 days after tumor inoculation, mice were divided into a control and three experimental groups, and then treated with rejT-iC9-EBV, rejT-NTEBV or original EBVCTL. FIG. 2B shows a graph showing that tumor signals progressively increased in mice without treatment, whereas tumor signals declined in mice treated with original EBVCTL. iC9-iPS derived-EBV CTLs also suppressed tumor signals equal to or greater than the original EBVCTL1.

FIG. 3A shows in vivo bioluminescent imaging of rejT-iC9-EBV expressing FFluc. NOD-Scid mice inoculated intraperitoneally with EBC-LCL cells and with rejT-iC9-EBV cells received three doses of CID (50 mg) intraperitoneally (n=4). Comparison mice received no CID (n=3). Images of three representative mice from each group are shown. FIG. 3B shows a plot of the bioluminescent T cell signal versus time for the NOD-Scid mice that received the CID and the comparison mice that received no CID.

FIG. 4 shows EGFR/KRAS neo-antigen candidates.

FIG. 5 shows results of cytotoxicity assays of CTLs specific for mutated KRAS G12V antigens. The % specific lysis for KRASG12V mutated clones at various effector:target ratios is shown.

FIGS. 6A and 6B show schematics of the strategy for isolating rare neoantigen-reactive T cells from peripheral blood. FIG. 6A shows isolation of CD8+CTLs, which are mixed with an artificial antigen presenting cell presenting a neo-antigen peptide. FIG. 6B shows FACS isolation and expansion of the neo-antigen-specific CTLs.

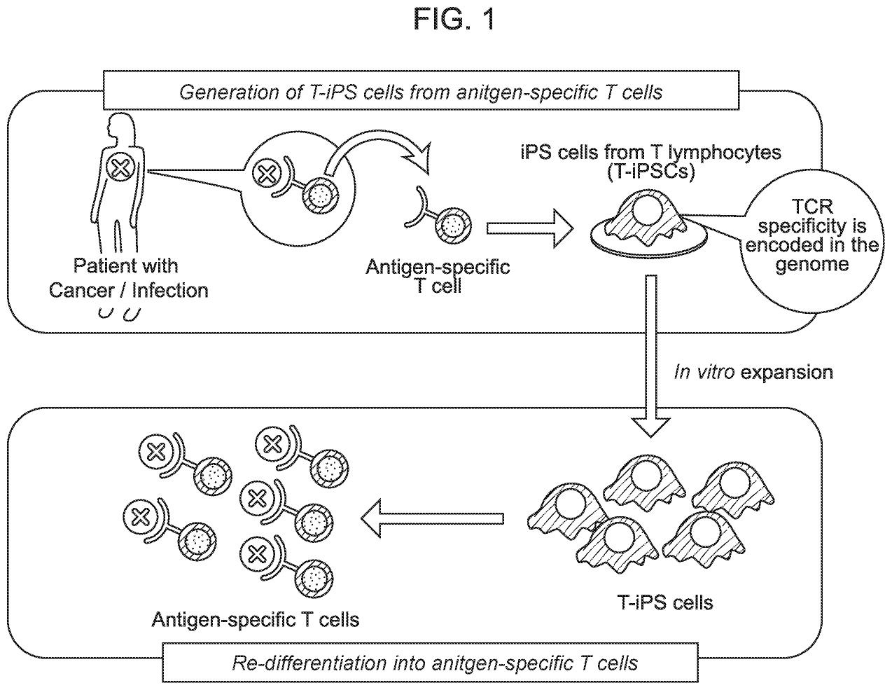

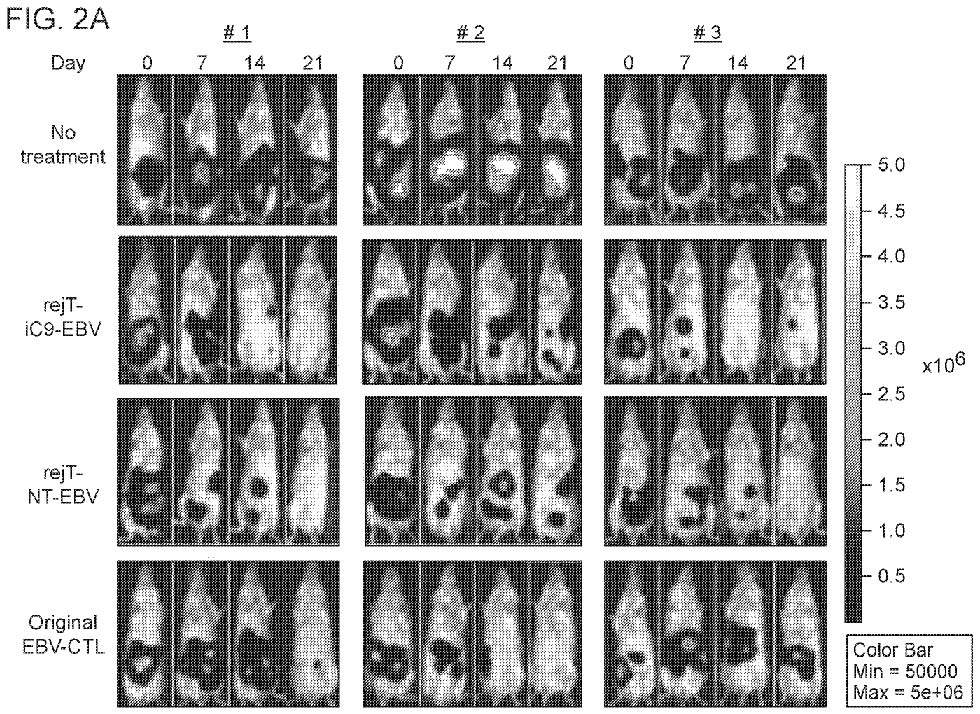

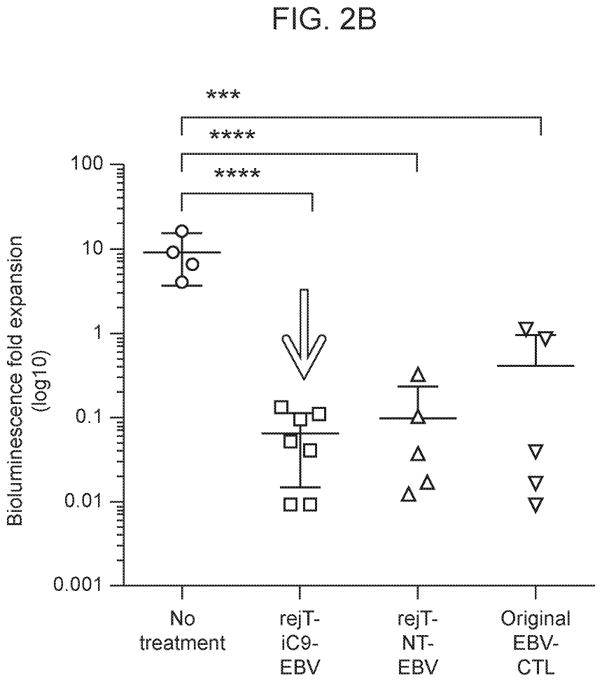

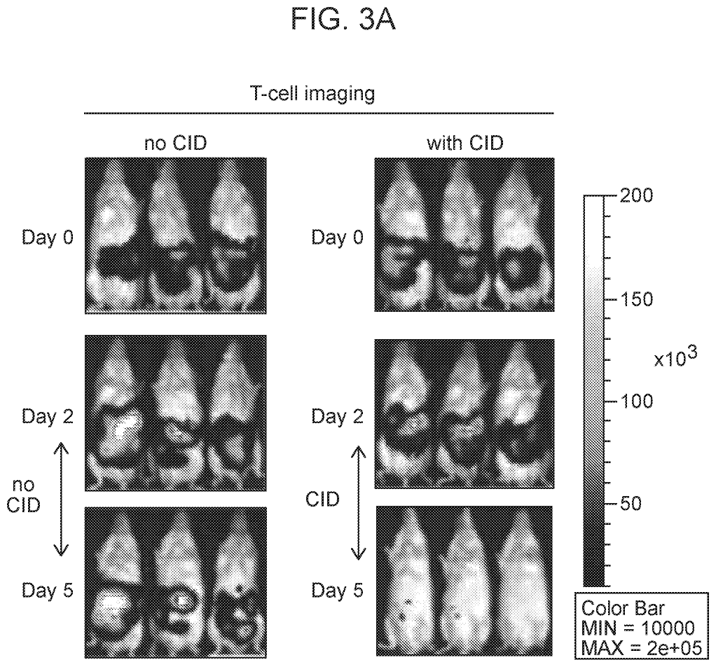

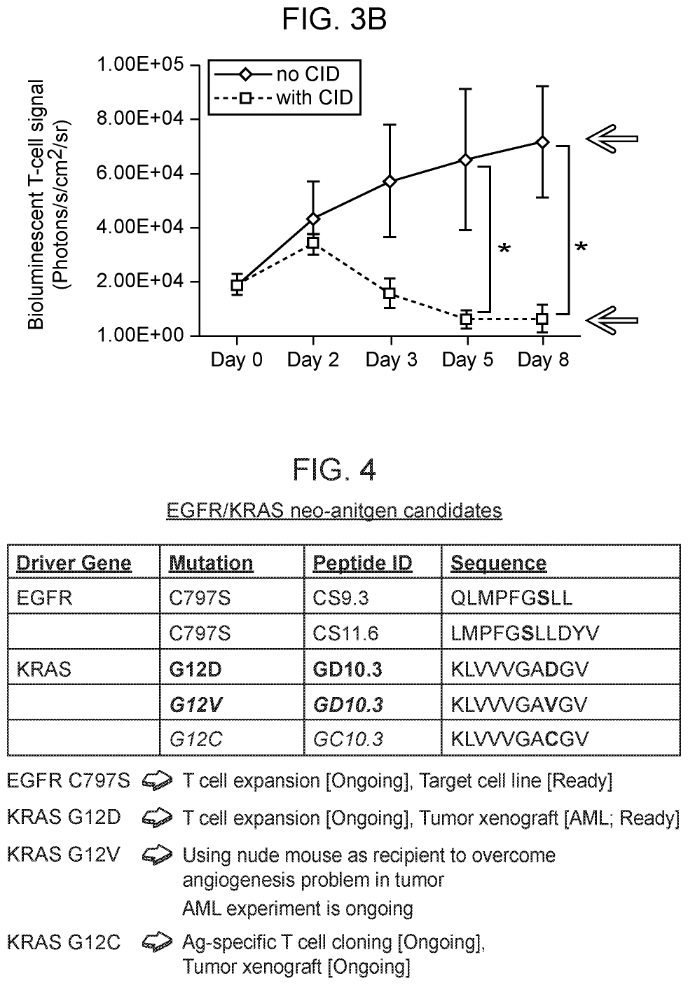

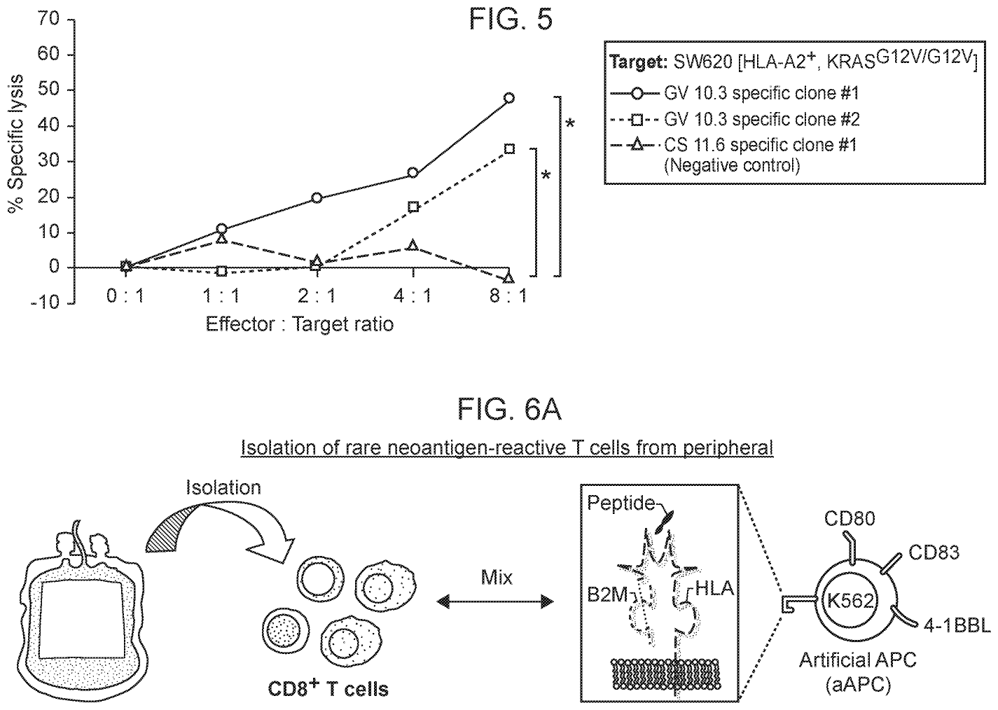

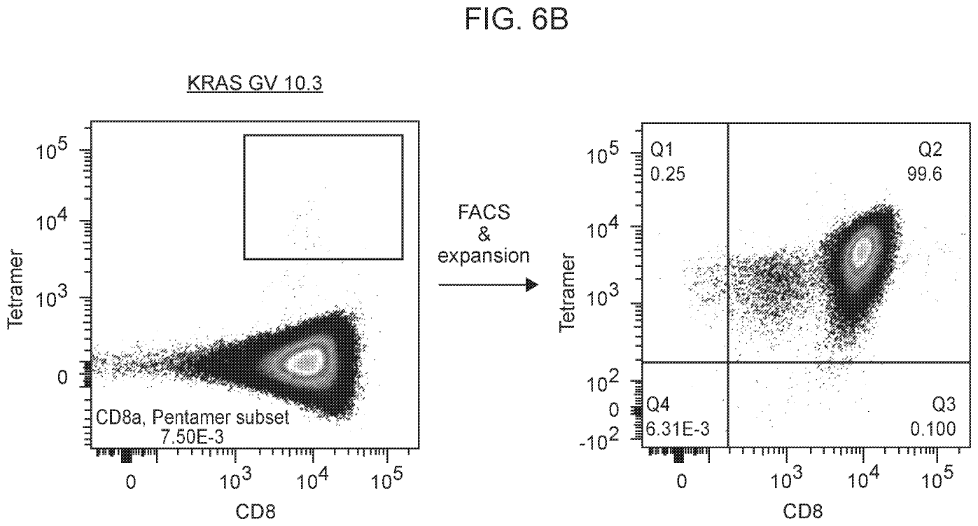

### DETAILED DESCRIPTION OF EMBODIMENTS

Compositions, methods, and kits are provided for producing IPSC-derived rejuvenated CTLs specific for mutated neo-antigen epitopes expressed on cancerous cells, including EGFR and KRAS neo-antigen epitopes. Also provided are pharmaceutical compositions comprising such IPSO-derived rejuvenated CTLs and methods of using them for treating cancers expressing the mutated neo-antigen epitopes.

Before the present compositions, methods, and kits are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Biological sample. The term “sample” with respect to an individual encompasses any sample comprising CTLs such as blood and other liquid samples of biological origin, solid tissue samples such as a biopsy specimen or cancerous tissue from a surgically resected tumor, malignant effusion fluid samples, or tissue cultures or cells derived or isolated therefrom, and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

The term “biological sample” encompasses a clinical sample. The types of “biological samples” include, but are not limited to: tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like.

The terms “obtained” or “obtaining” as used herein can also include the physical extraction or isolation of a biological sample (e.g., comprising CTLs) from a subject. Accordingly, a biological sample can be isolated from a subject (and thus “obtained”) by the same person or same entity that subsequently produces IPSO-derived rejuvenated CTLs from the CTLS in the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

By “immunogenic fragment” is meant a fragment of an immunogen which includes one or more epitopes that can stimulate an immune response, including an antigen-specific cytotoxic T cell response. Immunogenic peptides will typically range between 2 to 15 amino acids in length, including any length within this range such as 2, 3, 4, 5, 6, 7, 8, 9, 10, 11, 12, 13, 14, or 15 amino acids in length. In some embodiments, the immunogenic peptide is at least 2, at least 3, at least 5, at least 7, at least 9, at least 10, at least 11, or at least 12 amino acids in length.

As used herein, the term “epitope” generally refers to the site on an antigen which is recognized by a T-cell receptor (e.g., on a CTL) and/or an antibody. The epitope may be contained in a short peptide derived from a protein antigen or part of a protein antigen. Several different epitopes may be carried by a single antigenic molecule. The term “epitope” may also include modified amino acids. Epitopic determinants usually consist of chemically active surface groupings of molecules such as amino acids or sugar side chains and usually have specific three-dimensional structural characteristics, as well as specific charge characteristics.

An immunogenic fragment can be generated from knowledge of the amino acid and corresponding DNA sequences of an antigen (e.g., EGFR or KRAS), as well as from the nature of particular amino acids (e.g., size, charge, etc.) and the codon dictionary, without undue experimentation. See, e.g., Ivan Roitt, Essential Immunology, 1988; Kendrew, supra; Janis Kuby, Immunology, 1992 e.g., pp. 79-81. Some guidelines in determining whether a protein will stimulate a response, include: Peptide length—typically the peptide is about 8 or 9 amino acids long to fit into a MHC class I complex and about 13-25 amino acids long to fit into a class II MHC complex. Peptides may be longer than these lengths. For example, a longer peptide may be needed if it is partially degraded in cells. The peptide may contain an appropriate anchor motif which will enable it to bind to various class I or class II molecules with high enough specificity to generate an immune response (See Bocchia, M. et al, Specific Binding of Leukemia Oncogene Fusion Protein Pentides to HLA Class I Molecules, Blood 85:2680-2684; Englehard, V H, Structure of peptides associated with class I and class II MHC molecules Ann. Rev. Immunol. 12:181 (1994)).

The terms “immunogenic” protein or peptide refer to an antigen having an amino acid sequence which elicits an immunological response, including an antigen-specific cytotoxic T cell response. An “immunogenic” protein or peptide, as used herein, includes the full-length sequence of the protein in question, including the precursor and mature forms, analogs thereof, or immunogenic fragments thereof.

As used herein, the term “CTL epitope” refers generally to those features of a peptide structure which are capable of inducing a CTL response.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T cells (CTLs). CTLs have specificity for peptide antigens that are presented in association with proteins encoded by the major histocompatibility complex (MHC) and expressed on the surfaces of cells. CTLs help induce and promote the destruction or lysis of cancerous cells, infected cells, or damaged cells. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

The ability of a particular antigen to stimulate a cell-mediated immunological response may be determined by a number of assays, such as by lymphoproliferation (lymphocyte activation) assays, CTL cytotoxic cell assays (e.g., the interferon-γ (IFN-γ) enzyme-linked immune spot (ELISPOT) assay for measuring IFN-γ secretion from activated CTLs, the calcein release assay for measuring CTL cytotoxicity using calcein to label target cells, intracellular cytokine staining, granzyme B release assay, chromium release assay, JAM test, CD107a mobilization assay, caspase 3 assay, flow cytometric CTL assay) or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Methods of measuring a cell-mediated immune response include measurement of intracellular cytokines or cytokine secretion by T-cell populations, or by measurement of epitope specific T-cells (e.g., by the tetramer technique) (reviewed by Malyguine et al. (2012) Cells 1(2):111-126, Shafer-Weaver et al. (2003) J. Transl. Med. 1(1):14, Takagi et al. (2017) Biochem. Biophys. Res. Commun. 492(1):27-32, Jerome et al. (2003) Apoptosis 8(6):563-571, Hermans et al. (2004) J. Immunol. Methods 1; 285(1):25-40, van Baalen et al. (2008) Cytometry A 73(11):1058-1065, McMichael and O'Callaghan (1998) J. Exp. Med. 187(9)1367-1371, Mcheyzer-Williams et al. (1996) Immunol. Rev. 150:5-21, Lalvani et al. (1997) J. Exp. Med. 186:859-865; herein incorporated by reference.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with increased susceptibility to cancer, those suspected of having cancer, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, lung cancer, including non-small-cell lung carcinoma (e.g., adenocarcinoma, squamous-cell carcinoma and large-cell carcinoma) and small-cell lung carcinoma, breast cancer, prostate cancer, ovarian cancer, testicular cancer, colon cancer, pancreatic cancer, gastric cancer, hepatic cancer, leukemia, lymphoma, adrenal cancer, thyroid cancer, pituitary cancer, renal cancer, brain cancer, skin cancer, head cancer, neck cancer, oral cavity cancer, tongue cancer, and throat cancer.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “specific binding,” “specifically binds,” and the like, refer to non-covalent or covalent preferential binding to a molecule relative to other molecules or moieties in a solution or reaction mixture (e.g., specific binding to a particular peptide or epitope relative to other available peptides, such as binding of a CTL T cell receptor to an immunogenic peptide or CTL epitope presented by MHC on an antigen presenting cell). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

### Methods

Compositions, methods, and kits are provided for producing rejuvenated cytotoxic T cells (CTLs) specific for mutated neo-antigen epitopes expressed on cancerous cells, including epidermal growth factor receptor (EGFR) and KRAS neo-antigen epitopes. Antigen-specific CTLs are rejuvenated by reprogramming them into induced pluripotent stem cells (IPSCs) using Yamanaka factors and redifferentiating them back into CTLs while expanding their numbers. After redifferentiation, the IPSC-derived rejuvenated CTLs retain the antigen specificity of the original CTLs from which they were derived, but have the advantage of having longer telomeres and higher proliferative activity than the original CTLs. Pharmaceutical compositions comprising such IPSC-derived rejuvenated CTLs are useful for treating cancers expressing the mutated neo-antigen epitopes recognized by the original CTLs.

Immunogenic peptides comprising mutated neo-antigen epitopes may be used to elicit antigen-specific CTLs from either healthy individuals or from cancer patients. CTL responses are induced by contacting CTLs with an antigen presenting cell presenting at its surface the immunogenic peptide comprising the mutated neo-antigen epitope in a complex with major histocompatibility complex (MHC). At least one round of stimulation of the CTLs with the immunogenic peptide will be performed to generate a CTL response in order to provide antigen-specific CTLs that recognize a mutated neo-antigen epitope. In some embodiments, multiple rounds of stimulation of the CTLs with the immunogenic peptide may be performed to generate a CTL response capable of producing sufficient antigen-specific CTLs for processing to produce IPSC-derived rejuvenated antigen-specific CTLs for immunotherapy, as described further below. In certain embodiments, at least 2, at least 3, at least 4, at least 5, at least 6, at least 7, or at least 8 rounds or more of stimulation of the CTLs with an immunogenic peptide are performed.

Stimulation of CTLs with an immunogenic peptide (in the presence of an antigen presenting cell) can be performed in vivo, ex vivo, or in vitro. For example, the immunogenic peptide can be administered to a subject to elicit a CTL response followed by collection of a biological sample from the subject comprising antigen-specific CTLs recognizing mutated neo-antigen epitopes. The biological sample may be any sample containing CTLs specific for the mutated neo-antigen epitope such as a blood sample, a sample of peripheral blood mononuclear cell (PBMCs), cancerous tissue in which the CTLS have infiltrated, or a malignant effusion fluid sample. The CTLs can be isolated from a bodily fluid (e.g., blood) or tissue and cultured.

Alternatively, a biological sample comprising CTLs can be collected from a subject and treated with an immunogenic peptide in the presence of an antigen-presenting cell ex vivo or in vitro to generate antigen-specific CTLs. Examples of suitable antigen presenting cells that can present an immunogenic peptide to CTLs include dendritic cells, macrophages, and activated B cells. Alternately, artificial antigen presenting cells may be used, such as soluble MHC-multimers or cellular or acellular artificial antigen presenting cells. MHC-multimers typically range in size from dimers to octamers (tetramers commonly used) and can be used to display class 1 or class 2 MHC (Hadrup et al. (2009) Nature Methods 6:520-526, Nepom et al. (2003) Antigen 106:1-4, Bakker et al. (2005) Current Opinion in Immunology 17:428-433). Cellular artificial antigen presenting cells may include cells that have been genetically modified to express T-cell co-stimulatory molecules, MHC alleles and/or cytokines. For example, artificial antigen presenting cells have been generated from fibroblasts modified to express HLA molecules, the co-stimulatory signal, B7.1, and the cell adhesion molecules, ICAM-1 and LFA-3 (Latouche et al. (2000) Nature Biotechnology. 18 (4):405-409). Acellular antigen presenting cells comprise biocompatible particles such as microparticles or nanoparticles that carry T cell activating proteins on their surface (Sunshine et al. (2014) Biomaterials. 35 (1): 269-277), Perica et al. (2014) Nanomedicine: Nanotechnology, Biology and Medicine. 10 (1):119-129). For a review of artificial antigen presenting cells, see, e.g., Oelke et al. (2004) Clin. Immunol. 110(3):243-251, Wang et al. (2017) Theranostics. 7(14):3504-3516, Butler et al. (2014) Immunol Rev. 257(1):191-209, Eggermont et al. (2014) Trends Biotechnol. 32(9):4564-4565, Sunshine et al. (2013) Nanomedicine (Lond) 8(7):1173-1189, and Rhodes et al. (2018) Mol. Immunol. 98:13-18; herein incorporated by reference.

Typically, the immunogenic peptide is at a concentration ranging from about 10 μg/ml to about 40 μg/ml in the biological sample. The immunogenic peptide may be pre-incubated with the antigen presenting cells for periods ranging from 1 to 18 hours prior to stimulation of the CTLs. Culture media may be supplemented with interleukin 2 (IL-2) and interleukin 15 (IL-15) during intervals between stimulations to induce amino acid uptake and protein synthesis in antigen-activated T cells to promote growth and proliferation of antigen-specific CTLs. The antigen-specific CTLs can subsequently be isolated from biological samples, reprogrammed into induced pluripotent stem cells, and redifferentiated into IPSO-derived rejuvenated CTLs that are specific for the mutated neo-antigen epitope recognized by the original CTLs.

Neoantigens include tumor-associated antigens that are not present in the normal human genome. Immunogenic peptides may include mutated epitopes of neoantigens that are expressed by cancerous cells from any form of cancer including malignant, metastatic and non-metastatic types of cancer, at any stage (I, II, III, IV or V) or grade (G1, G2, G3, etc.), including carcinomas, such as squamous cell carcinoma, adenocarcinoma, adenosquamous carcinoma, anaplastic carcinoma, large cell carcinoma, and small cell carcinoma. In certain embodiments, immunogenic peptides include mutated epitopes of neoantigens expressed by lung cancer, including non-small-cell lung carcinoma (e.g., adenocarcinoma, squamous-cell carcinoma and large-cell carcinoma) and small-cell lung carcinoma, breast cancer, prostate cancer, ovarian cancer, testicular cancer, colon cancer, pancreatic cancer, gastric cancer, hepatic cancer, leukemia, lymphoma, adrenal cancer, thyroid cancer, pituitary cancer, renal cancer, brain cancer, skin cancer, head cancer, neck cancer, oral cavity cancer, tongue cancer, and throat cancer.

An immunogenic peptide comprising a mutated neoantigen CTL epitope can be designed based on knowledge of the amino acid sequence of the mutated neoantigen of interest (e.g., expressed by a cancer in a patient undergoing treatment). Typically, the immunogenic peptide will range in size from 8-12 amino acids in length (i.e., in order to fit into the MHC class I complex for presentation to CTLs), though immunogenic peptides may be longer, particularly if the immunogenic peptide is degraded in cells or the biological sample. The immunogenic peptide may further contain an appropriate anchor motif which will enable it to bind to various MHC class I or class II molecules with high enough specificity to generate an immune response (See Bocchia, M. et al, Specific Binding of Leukemia Oncogene Fusion Protein Pentides to HLA Class I Molecules, Blood 85:2680-2684; Englehard, V H, Structure of peptides associated with class I and class II MHC molecules Ann. Rev. Immunol. 12:181 (1994)). The sequence of a neoantigen of interest can be compared to published structures of peptides associated with MHC molecules. Representative MHC binding peptides can be found in a number of databases including, the MHCBN, JenPep, MHCPEP, and SYFPEITHI databases. In addition, epitope prediction software can be used for prediction of MHC binding peptides and CTL epitopes for various MHC alleles. For example, nHLAPred (crdd.osdd.net/raghava/nhlapred/) uses artificial neural networks (ANNs) and quantitative matrices (QM) for prediction of MHC binding peptides and CTL epitopes for various MHC alleles. ProPredl (crdd.osdd.net/raghava/propredl/) identifies MHC Class-I binding regions in antigens for MHC Class-I alleles. BIMAS (bimas.cit.nih.gov/molbio/hla_bind/, Lefranc et al. (2003) Leukemia 17:260-266) predicts MHC-binding peptides based on their predicted half-time of dissociation from MHC class I alleles. RANKPEP ranks peptides based on their sequences according to their similarity to peptides known to bind to a given MHC molecule. PREDEP (margalit.huji.ac.il/Teppred/mhc-bind/) is a structure-based algorithm for prediction of MHC class I epitopes. MMBPred (crdd.osdd.net/raghava/mmbpred/) predicts mutated MHC class-I binding peptides in antigenic proteins for MHC class I alleles.

In certain embodiments, the immunogenic peptide comprises a mutated EGFR neo-antigen epitope comprising a mutation selected from the group consisting of a C797S mutation, a T790M mutation, an L858R mutation, and a deletion. In other embodiments, the immunogenic peptide comprises a mutated KRAS neo-antigen epitope comprising a mutation selected from the group consisting of a G12D mutation, a G12V mutation, and a G12C mutation. In certain embodiments, the immunogenic peptide comprises an amino acid sequence selected from the group consisting of SEQ ID NOS:1-5, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% sequence identity thereto, wherein the immunogenic peptide comprises a mutated EGFR or KRAS neo-antigen epitope.

The ability of a particular immunogenic peptide to stimulate a CTL cell-mediated immunological response may be determined by a number of assays, such as by lymphoproliferation (lymphocyte activation) assays, CTL cytotoxic cell assays (e.g., the interferon-γ (IFN-γ) enzyme-linked immune spot (ELISPOT) assay for measuring IFN-γ secretion from activated CTLs, the calcein release assay for measuring CTL cytotoxicity using calcein to label target cells, intracellular cytokine staining, granzyme B release assay, chromium release assay, JAM test, CD107a mobilization assay, caspase 3 assay, flow cytometric CTL assay) or by assaying for CTLs specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Methods of measuring a cell-mediated immune response include measurement of intracellular cytokines or cytokine secretion by T-cell populations, or by measurement of epitope specific T-cells (e.g., by the tetramer technique) (reviewed by Malyguine et al. (2012) Cells 1(2):111-126, Shafer-Weaver et al. (2003) J. Transl. Med. 1(1):14, Takagi et al. (2017) Biochem Biophys Res Commun. 492(1):27-32, Jerome et al. (2003) Apoptosis 8(6):563-571, Hermans et al. (2004) J. Immunol. Methods 1; 285(1):25-40, van Baalen et al. (2008) Cytometry A 73(11):1058-1065, McMichael and O'Callaghan (1998) J. Exp. Med. 187(9)1367-1371, Mcheyzer-Williams et al. (1996) Immunol. Rev. 150:5-21, Lalvani et al. (1997) J. Exp. Med. 186:859-865; herein incorporated by reference.

The antigen-specific CTLs can optionally be purified before or after reprogramming and redifferentiation by any method known in the art, including, but not limited to, density gradient centrifugation (e.g., Ficoll Hypaque, percoll, iodoxanol and sodium metrizoate), immunoselection (positive selection or negative selection for surface markers) with immunomagnetic beads or immunoaffinity columns, or fluorescence-activated cell sorting (FACS). See, e.g., Cytotoxic T-Cells, Methods and Protocols (E. Ranieri, ed., Humana Press, 2014), Thiery et al. (2010) Curr. Protoc. Cell Biol. Chapter 3:Unit 3.37, and Oelke et al. (2000) Clin. Cancer Res. 6(5):1997-2005; herein incorporated by reference.

### Reprogramming Antigen-Specific Cytotoxic T Cells

Rejuvenated antigen-specific CTLs and be generated by reprogramming the original CTLs obtained from a subject into pluripotent stem cells followed by redifferentiation. CTLs are induced into forming pluripotent stem cells, for example, by treating them with reprograming factors such as Yamanaka factors, including but not limited to, OCT3, OCT4, SOX2, KLF4, c-MYC, NANOG, and LIN28 (see, e.g., Nishimura et al. (2013) Cell Stem Cell 12:114-126, Takayama et al. (2010) J. Exp. Med. 207:2817-2830, and U.S. Pat. No. 9,206,394; herein incorporated by reference in their entireties). After in vitro expansion, the CTL-derived IPSCs can be redifferentiated into hematopoietic cells by culturing the IPSCs in the presence of VEGF, SCF, and FLT-3L. The hematopoietic cells can subsequently be redifferentiated into CTLs expressing a desired T cell receptor by culturing in the presence of FLT-3L and IL-7. After such redifferentiation, the CTLs are now rejuvenated (i.e., IPSC-derived rejuvenated CTLs have longer telomeres and higher proliferative activity than the original CTLs while retaining the specificity for neo-antigen epitopes recognized by the original CTLs). For redifferentiation protocols, see, e.g., Nishimura et al., supra; Takayama et al., supra; Timmermans et al. (2009) J. Immunol. 182:6879-6888, and Ikawa et al. (2010) Science 329:93-96; herein incorporated by reference in their entireties.

Methods for “introducing a cell reprogramming factor into CTLs are not limited in particular, and known procedures can be selected and used as appropriate. For example, when a cell reprogramming factor as described above is introduced into CTLs of the above-mentioned type in the form of proteins, such methods include ones using protein introducing reagents, fusion proteins with protein transfer domains (PTDs), electroporation, and microinjection. When a cell reprogramming factor as described above is introduced into CTLs of the above-mentioned type in the form of nucleic acids encoding the cell reprogramming factor, a nucleic acid(s), such as cDNA(s), encoding the cell reprogramming factor can be inserted in an appropriate expression vector comprising a promoter that functions in CTLs, which then can be introduced into CTLs by procedures such as infection, lipofection, liposomes, electroporation, calcium phosphate coprecipitation, DEAE-dextran, microinjection, and electroporation.

Examples of an “expression vector” include viral vectors, such as lentiviruses, retroviruses, adenoviruses, adeno-associated viruses, and herpes viruses; and expression plasmids for animal cells. For example, retroviral or Sendai virus (SeV) vectors are commonly used to introduce a nucleic acid(s) encoding a cell reprogramming factor as described above into CTLs.

In addition, a suicide gene may be introduced into the IPSO-derived rejuvenated CTLs, for example, to improve their safety by allowing their destruction at will. Suicide genes can be used to selectively kill cells by inducing apoptosis or converting a nontoxic drug to a toxic compound in the CTLs. Examples include suicide genes encoding caspases, thymidine kinases, cytosine deaminases, intracellular antibodies, telomerases, and DNases. See, e.g., Jones et al. (2014) Front. Pharmacol. 5:254, Mitsui et al. (2017) Mol. Ther. Methods Clin. Dev. 5:51-58, Greco et al. (2015) Front. Pharmacol. 6:95; herein incorporated by reference. In some cases, the suicide gene is expressed from an inducible promoter to provide a “safety switch” (i.e., kill cells by inducing the suicide gene). For example, an inducible caspase-9 suicide gene system can be incorporated into IPSO-derived rejuvenated CTLs as a “safety switch” (see, e.g., Straathof et al. (2005) Blood 105(11):4247-4254; Thomis et al. (2001) Blood 97(5):1249-1257; Tey et al. (2007) Biol. Blood Marrow Transplant. 13(8):913-24; herein incorporated by reference.). In some embodiments, a suicide gene is selected that expresses a human protein to minimize immune reactions in human patients treated with the CTLs.

Pharmaceutical Compositions and Cellular Immunotherapy with Rejuvenated CTLs

Pharmaceutical compositions can be prepared by formulating the IPSO-derived rejuvenated CTLs produced by the methods described herein into dosage forms by known pharmaceutical methods. For example, a pharmaceutical composition comprising IPSC-derived rejuvenated CTLs can be formulated for parenteral administration, as capsules, liquids, film-coated preparations, suspensions, emulsions, and injections (such as venous injections, drip injections, and the like).

In formulation into these dosage forms, the IPSO-derived rejuvenated CTLs can be combined as appropriate, with pharmaceutically acceptable carriers or media, in particular, sterile water and physiological saline, vegetable oils, resolvents, bases, emulsifiers, suspending agents, surfactants, stabilizers, vehicles, antiseptics, binders, diluents, tonicity agents, soothing agents, bulking agents, disintegrants, buffering agents, coating agents, lubricants, coloring agents, solution adjuvants, or other additives. The IPSO-derived rejuvenated CTLs may be also used in combination with known pharmaceutical compositions, immunostimulants, anti-cancer agents, or other therapeutic agents.

In some embodiments, the pharmaceutical composition comprising the IPSO-derived rejuvenated CTLs is a sustained-release formulation, or a formulation that is administered using a sustained-release device. Such devices are well known in the art, and include, for example, transdermal patches, and miniature implantable pumps that can provide for delivery of the IPSO-derived rejuvenated CTLs over time in a continuous, steady-state fashion at a variety of doses to achieve a sustained-release effect with a non-sustained-release pharmaceutical composition.

Usually, but not always, the subject who receives the IPSO-derived rejuvenated CTLs (i.e., the recipient) is also the subject from whom the original CTLs (i.e., before rejuvenation) are harvested or obtained, which provides the advantage that the cells are autologous. However, CTLs can be obtained from another subject (i.e., donor), a culture of cells from a donor, or from established cell culture lines and rejuvenated according to the methods described herein. CTLs may be obtained from the same or a different species than the subject to be treated, but preferably are of the same species, and more preferably of the same immunological profile as the subject. Such cells can be obtained, for example, from a biological sample comprising CTLs from a close relative or matched donor, then reprogrammed into induced pluripotent stem cells (IPSCs) using Yamanaka factors and redifferentiated into the IPSO-derived rejuvenated CTLs and administered to a subject in need of treatment for cancer.

In certain embodiments, the IPSC-derived rejuvenated CTLs administered to a subject are autologous or allogeneic. The patients or subjects who donate or receive the CTLs are typically mammalian, and usually human. However, this need not always be the case, as veterinary applications are also contemplated.

At least one therapeutically effective dose of the IPSC-derived rejuvenated CTLs will be administered. By “therapeutically effective dose or amount” of the IPSC-derived rejuvenated CTLs is intended an amount that when administered brings about a positive therapeutic response with respect to treatment of an individual for cancer. Of particular interest is an amount of the IPSC-derived rejuvenated CTLs that provides an anti-tumor effect, as defined herein. By “positive therapeutic response” is intended the individual undergoing the treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

The pharmaceutical compositions comprising the IPSC-derived rejuvenated CTLs may be administered using any route of administration in accordance with any medically acceptable method known in the art. Suitable routes of administration include parenteral administration, such as intravenous (IV), intraarterial, infusion, subcutaneous (SC), intraperitoneal (IP), intramuscular (IM), pulmonary, nasal, topical, or transdermal. In some embodiments, the pharmaceutical composition comprising the IPSC-derived rejuvenated CTLs is administered locally to the site of a tumor or cancerous cells.

Factors influencing the respective amount of the various compositions to be administered include, but are not limited to, the mode of administration, the frequency of administration, the particular type of cancer undergoing therapy, the severity of the disease, the history of the disease, whether the individual is undergoing concurrent therapy with another therapeutic agent, and the age, height, weight, health, and physical condition of the individual undergoing therapy. Generally, a higher dosage is preferred with increasing weight of the subject undergoing therapy.

In certain embodiments, multiple therapeutically effective doses of the IPSC-derived rejuvenated CTLs will be administered for a time period sufficient to effect at least a partial tumor response and more preferably a complete tumor response. Where a subject undergoing immunotherapy exhibits a partial response, or a relapse following a prolonged period of remission, subsequent courses of immunotherapy may be needed to achieve complete remission of the disease. Thus, subsequent to a period of time off from a first treatment period, a subject may receive one or more additional treatment periods comprising immunotherapy with IPSO-derived rejuvenated CTLs. Such a period of time off between treatment periods is referred to herein as a time period of discontinuance. It is recognized that the length of the time period of discontinuance is dependent upon the degree of tumor response (i.e., complete versus partial) achieved with any prior treatment periods of immunotherapy with the IPSC-derived rejuvenated CTLs or other therapeutic agents.

### Kits

Also provided are kits for practicing the methods described herein. In certain embodiments, the kit comprises IPSO-derived rejuvenated CTLs specific for a mutated EGFR or KRAS neo-antigen epitope or reagents for preparing them. For example, the kit may comprise an immunogenic peptide comprising a mutated EGFR or KRAS neo-antigen epitope, an antigen presenting cell (e.g., dendritic cell, macrophage, or cellular or acellular artificial antigen presenting cell (e.g., MHC multimer)), agents for isolating CTLs (e.g., immunomagnetic beads or immunoaffinity columns), reprograming factors (e.g., Yamanaka factors such as OCT3, OCT4, SOX2, KLF4, c-MYC, NANOG, and LIN28), redifferentiation factors (e.g., VEGF, SCF, FLT-3L, and IL-7), and culture media. Alternatively, the kit may comprise IPSO-derived rejuvenated CTLs specific for a mutated EGFR or KRAS neo-antigen epitope in a pharmaceutical composition suitable for use in treatment.

In certain embodiments, the kit comprises an immunogenic peptide comprising an amino acid sequence selected from the group consisting of SEQ ID NOS:1-5, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity thereto, wherein the immunogenic peptide comprises a mutated EGFR or KRAS neo-antigen epitope.

Kits may comprise one or more containers of the compositions described herein. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the IPSC-derived rejuvenated CTLs.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Utility

The IPSO-derived rejuvenated CTLs, produced by the methods described herein, are useful in cellular immunotherapy for treating cancer, particularly cancers expressing mutated EGFR or KRAS neo-antigen epitopes.

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

The term “cancer” as used herein includes carcinomas, (e.g., carcinoma in situ, invasive carcinoma, metastatic carcinoma) and pre-malignant conditions, i.e. neomorphic changes independent of their histological origin. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included.

Cancers and cancer cells that can be treated with IPSO-derived rejuvenated CTLs include, but are not limited to, hematological cancers, including leukemia, lymphoma and myeloma, and solid cancers, including for example tumors of the brain (glioblastomas, medulloblastoma, astrocytoma, oligodendroglioma, ependymomas), carcinomas, e.g. carcinoma of the lung, liver, thyroid, bone, adrenal, spleen, kidney, lymph node, small intestine, pancreas, colon, stomach, breast, endometrium, prostate, testicle, ovary, skin, head and neck, and esophagus; sarcomas, melanomas; myelomas; etc.

In particular, lung cancer may be responsive to treatment with IPSO-derived rejuvenated CTLs specific for mutated EGFR or KRAS neo-antigen epitopes including, without limitation, non-small-cell lung carcinoma (e.g., adenocarcinoma, squamous-cell carcinoma and large-cell carcinoma) and small-cell lung cancer. In an embodiment, the lung cancer is non-small cell lung carcinoma (NSCLC). In certain embodiments, the NSCLC comprises a mutated EGFR neo-antigen comprising a mutation selected from the group consisting of a C797S mutation, a T790M mutation, an L858R mutation, and a deletion. In other embodiments, the NSCLC comprises a mutated KRAS neo-antigen comprising a mutation selected from the group consisting of a G12D mutation, a G12V mutation, and a G12C mutation.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Introduction

The discovery of iPSC technology created promising new avenues for treatment^{2,3,4,5,6,7}. Human iPSCs are a potential source of stem cells for transfusion therapies. The development of widely varying reprogramming methods has enabled us nowadays to obtain iPSCs from even a small number of antigen-specific T cells of patient origin. As these T cell-derived iPSCs (T-iPSCs) carry TCR gene rearrangements in their genomic DNA, they are likely useful for producing antigen-specific T cells and for studying T cell development. T cell immunotherapy is potentially an effective therapeutic strategy against many types of cancers and viral infections. If antigen-specific T cells tailored against diseases and for patients can be easily obtained, T cell immunotherapy should become a popular choice of therapy. However, use of T cell immunotherapy is restricted by HLA alleles. In addition, expansion of CTLs ex vivo has hitherto resulted in production of CTLs with short telomeres and an “exhausted” phenotype. Our laboratory developed an in vitro way to reprogram antigen-specific CTLs to T-iPSCs for expansion and then to guide them sequentially to yield T-lineage cells and mature CD8 single-positive T cells. These CD8+ T cells generated in vitro display antigen-specific cytotoxicity and enhanced proliferative capacity with longer telomeres. Since these T cells originate from a patient's own CTLs, HLA restriction is not an issue. This novel technique thus provides approaches to generate rejuvenated antigen-specific T cells in unlimited numbers. This discovery should resolve issues related to T cell adoptive immunotherapy both qualitatively and quantitatively.

T lymphocytes play a central role in acquired immunity and control systemic immunity against internal and external pathogens. CTLs and helper lymphocytes are important components of the immune system in the fight against cancers^{9,10}. These T lymphocytes start to exert their proliferative functions when, via TCRs, they recognize antigens in an HLA-restricted and antigen-specific manner. Adoptive T cell immunotherapy that exploits these features is evolving as a technology with the potential of providing ways safely and effectively to target pathogens for destruction. The greatest advantages of adoptive T cell immunotherapy lie in specific recognition of target cells and in long-term immunological surveillance by long-lived native T lymphocytes.

In fact, successful treatment of cancers with allogeneic T lymphocytes is a direct proof that human T-cell immunity has the potential to eradicate cancers. However, the effectiveness of adoptive T-cell immunotherapy is often hampered by insufficient recognition of cancer antigens (principally self-antigens), on cancer cells. It is also true that continuous exposure to cancer/self-antigens drives T lymphocytes into a highly exhausted state, with loss of potential for long-term survival, proliferation, and killing functions¹⁰. Several researchers have endeavored to develop clinical protocols for expanding antigen-responding T cells, i.e., tumor-infiltrating lymphocytes, from the few native T cell pools remaining in the patient. Highly expanded T cells in such protocols have not proved fully effective so far because of functional losses incurred during ex vivo manipulation. To overcome these obstacles in cell-based immunotherapy, we endeavored to generate iPS cells from a single T cell of a cancer patient. iPSCs have a capacity for unlimited self-renewal while maintaining pluripotency⁹. Unlike other somatic cell-derived iPS cells, TiPSCs have properly rearranged TCRs even after having undergone nuclear reprogramming.

### Example 2

### Rejuvenation of Antigen-Specific Cytotoxic T Cells

We have recently developed a novel system in which antigen-specific cytotoxic T cells (CTLs) can be rejuvenated by reprogramming them into induced pluripotent stem cells (iPSCs) and redifferentiating them while expanding their numbers, yielding abundant rejuvenated T cells (rejT cells) (Generation of rejuvenated antigen-specific T cells by reprogramming to pluripotency and redifferentiation. 2013 Jan. 3; 12(1):114-26. Cell Stem Cell). This unique technique has been deployed in vivo with a safeguard system as a model of iPSC-derived, rejCTL therapy (A safeguard system for induced pluripotent stem cell-derived rejuvenated T cell therapy. 2015 Oct. 13; 5(4):597-608. Stem Cell Report). The purpose of this innovative method, the first exploration of the concept of a “kill switch”, is to ensure that using iPSC-based therapy in humans is safe.

Adoptive T-cell immunotherapy has shown promise in treating melanoma and other cancers; however, cytotoxic T-cells can become exhausted, with loss of efficacy during ex vivo expansion. To overcome this obstacle, we have developed a novel system in which antigen-specific T cells are reprogrammed to pluripotent stem cells (T-iPSCs) using Yamanaka factors. After expansion, these T-iPSCs are redifferentiated to functional T cells. They retain their original antigen specificity. These newly redifferentiated T cells display a naïve T cell phenotype, with longer telomeres and higher proliferative activity. These iPSCs generated from human T lymphocytes (T-iPSCs) retain their T-cell receptor (TCR) specificity in the genome as encoded by rearranged TCR alpha and beta genes^{16,17,18}. Because T-iPS cells have unlimited self-renewal capacity, they can be expanded ex vivo. When these T-iPS cells are re-differentiated into CD3+TCR, they are newly generated T cells with original antigen specificity but longer telomeres^{11,8}. We have demonstrated killing activity and specificity of these “rejuvenated” T cells in vivo. In fact, rejCTL cells show more robust biological activity than the original T cells.

This unique approach differs from chimeric antigen receptor T-cell (CART) immunotherapy. It uses T cell receptors to recognize non-self-antigens/epitopes expressed inside tumor cells; therefore, this form of immunotherapy using rejCTL cells is restricted by the HLA type. The advantages include that 1) once a iPS cell line is established, T-iPSCs can be generated indefinitely from them, producing young and active T cells without limitation; 2) T-iPSCs can be frozen for future use in patients with the same HLA type and mutation profile; 3) a safeguard system using inducible caspase 9 (iCas9) can be activated to eliminate all T-iPSC-derived cells in case of immunotherapy-associated complications; 4) it offers the opportunity to search for yet unknown cancer epitopes by searching T-iPSC libraries generated from tumor-infiltrating T cells. Application of this technology in lung cancer treatment will open a novel avenue for translational cancer therapies.

T cells are superior to antibodies in that they can recognize antigenic epitopes inside target cells, epitopes that are presented utilizing the MHC-based system. A disadvantage is perhaps that their ability to recognize antigens is restricted by the MHC allotype. This MHC-based restriction has been an issue in T cell immunotherapy. However, the issue has been addressed by the recent development of iPSC technology enabling the generation of iPSCs from a patient's own T cells. Utilizing this technology, we have developed a system to rejuvenate exhausted CTLs through reprogramming and redifferentiation. This should permit novel adoptive immunotherapies for cancer and viral infections.

In addition, a safeguard system using iCas9 engineered iPSCs can be applied to any first-in-man study using iPSC- or embryonic stem cell (ESC)-derived cells. Patients' tumor infiltrating T cells can be used to make T-iPSC libraries, followed by clonal redifferentiation of CTLs to search for novel cancer epitopes. This approach may serve to identify yet unknown targets for future use in cancer immunotherapy.

### Example 3

### Antigen-Specific Cytotoxic T Cells Targeting Mutated Epitopes of EGFR

### Introduction

Mutation-associated epitopes of the receptor tyrosine kinase, EGFR, are commonly present in lung and other forms of cancer. In lung cancer, mutations that activate epidermal growth factor receptor (EGFR) are often found in exons 18 to 21 of EGFR, the portion of the gene that encodes the tyrosine kinase domain of EGFR protein. Exon 19 deletions and exon 21 point mutations account for around 90% of all EGFR mutations in advanced NSCLC. EGFR tyrosine kinase inhibitors (TKIs), such as gefitinib and erlotinib, show therapeutic efficacy against NSCLC when such EGFR mutations are present. However, patients frequently develop resistance to EGFR-TKIs with a secondary mutation, a threonine to methionine change at codon 790 of EGFR (EGFR T790M). This is the major mechanism of EGFR-TKI resistance. It causes cancer relapse. Secondary mutations that occur in EGFR are the main mechanism of resistance to tyrosine kinase inhibitors (TKI) active against primarily mutations of EGFR. Mutations in EGFR are often found in cancers arising outside the lung, such as in the pancreas or breast. Without being bound by theory, we propose that T-cell based immunotherapy will not only work effectively against lung cancer but also other solid tumors having the same EGFR mutations. In addition, this approach can also be applied to mutations in other genes associated with cancer (MET, IGF-1R, etc.). Collectively, development of rejuvenated T-iPSCs for lung cancer immunotherapy may have a broad impact on future iPSC-mediated clinical therapy of cancer.

Peptide Prediction and Synthesis, Based on HLA Alleles, of the Peptides Representing EGFR Mutations and Selection of Those with Highest Affinity by Peptide Binding Assay.

We use the epitope prediction software, BIMAS (bimas.cit.nih.gov/molbio/hla_bind/), to predict peptides that can bind to various HLA alleles^{10,11,12}.

Generation of Rejuvenated, iC9-Implemented CTLs In Vitro.

The entire process of generating rejCTLs can be divided into the following steps: A) Induction of CTLs specific to EFGR epitopes carrying mutations (e.g., T790M, deletion and L858R) that were selected by the in-silicon approach. Peripheral-blood mononuclear cells (PBMNCs) contain some mutant EGFR-specific T cells. Because mutant EGFR-specific T cells also infiltrate into primary lung cancer tissue, they can be isolated from such tissue and cultured. Selected peptides are used to treat the original T cells. Peptide-specific responsive T cells are selected using tetramer and FACS isolation. B) Generation of T-iPSCs from EGFR mutation-specific CTLs and implementation of a iC9 based safeguard system¹. The CTLs generated in A) are reprogrammed into T-iPSCs using Sendai virus. The iC9 system is implemented in the T-iPSCs. C) Redifferentiation of CTLs from T-iPSCs. After in vitro expansion, T-iPSCs are redifferentiated into abundant rejCTLs carrying inducible iC9. Antigen specificity, killing activity, and proliferation activity are assayed in vitro.

Evaluation of the Therapeutic Efficacy of the Series of rejCTLs Recognizing Different Mutant EGFR Epitopes.

Infusion of patient rejCTLs into tumor-grafted mice and evaluation of tumor regression (speed and completeness). The rejCTLs are injected intraperitoneally into patient tumor-grafted mice. Control CTLs, as originally isolated, are injected into xenografted mice to permit comparisons. Tumor sizes are monitored after rejCTL cell injection¹. Timely imaging is used to document changes in the tumor in vivo. The most efficient epitopes for EGFR mutant NSCLCs will demonstrate changes in tumor size in vivo.

### Example 4

### Antigen-Specific Cytotoxic T Cells Targeting NSCLC Mutated Tumor Epitopes

The immunogenicity of EGFR and Ras mutations found in NSCLC in association with various HLA alleles are evaluated, and CTLs specific to the mutation sequences are generated. By reprogramming and redifferentiating these NSCLC-specific CTLs, rejCTLs are obtained. In the presence of certain human leukocyte antigen (HLA) alleles, a mutated protein such as that in EGFR T790M-harboring cancer cells is presented as a tumor-specific antigen and is targeted by activated immune cells. We screen various EGFR and KRas mutations (Table 1) and assess their binding affinity to various HLA alleles; the immunogenicity of the mutation-derived peptide sequences with particular HLA molecules is tested in vitro, using transporter associated with antigen processing (TAP)-deficient cell lines.

TABLE 1
EGFR/KRAS Neo-Antigen Candidates

| Driver | | Peptide | |
|---|---|---|---|
| Gene | Mutation | ID | Peptide Sequence |
| EGFR | C797S | CS9.3 | QLMPFGSLL (SEQ ID NO: 1) |
| | C797S | CS11.6 | LMPFGSLLDYV (SEQ ID NO: 2) |
| KRAS | G12D | GD10.3 | KLVVVGADGV (SEQ ID NO: 3) |
| | G12V | GV10.3 | KLVVVGAVGV (SEQ ID NO: 4) |
| | G12C | GC10.3 | KLVVVGACGV (SEQ ID NO: 5) |

TABLE 2

| | CDR1 | CDR2 | CDR3 | Frequency |
|---|---|---|---|---|
| TCRalpha | DSVNN | IPSGT | AVDNYGQNFV | 770758 |
| Donor Y | (SEQ ID NO: 6) | (SEQ ID NO: 66) | (SEQ ID NO: 130) | |
| (CS9.3) | | | | |
| | DSVNN | IPSGT | AVGNYGQNFV | 2095 |
| | (SEQ ID NO: 6) | (SEQ ID NO: 66) | (SEQ ID NO: 131) | |
| | DSVNN | IPSGT | AVDSYGQNFV | 1969 |
| | (SEQ ID NO: 6) | (SEQ ID NO: 66) | (SEQ ID NO: 132) | |
| | DSVNN | IPSGT | AADNYGQNFV | 1942 |
| | (SEQ ID NO: 6) | (SEQ ID NO: 66) | (SEQ ID NO: 133) | |
| | DSVNN | IPSGT | AVDDYGQNFV | 1404 |
| | (SEQ ID NO: 6) | (SEQ ID NO: 66) | (SEQ ID NO: 134) | |
| | SSVSVY | YLSGSTLV | AVTFTGGGNKLT | 1129 |
| | (SEQ ID NO: 7) | (SEQ ID NO: 67) | (SEQ ID NO: 135) | |
| | DSVNN | IPSGT | AVNNYGQNFV | 736 |
| | (SEQ ID NO: 6) | (SEQ ID NO: 66) | (SEQ ID NO: 136) | |
| | TISGNEY | GLKNN | IVNWGSNSGYALN | 338 |
| | (SEQ ID NO: 8) | (SEQ ID NO: 68) | (SEQ ID NO: 137) | |
| | DSVNN | IPSGT | AEDNYGQNFV | 228 |
| | (SEQ ID NO: 6) | (SEQ ID NO: 66) | (SEQ ID NO: 138) | |
| | DSVNN | IPSGT | AVVNYGQNFV | 218 |
| | (SEQ ID NO: 6) | (SEQ ID NO: 66) | (SEQ ID NO: 139) | |
| | DSVNN | IPSGT | AVDIYGQNFV | 214 |
| | (SEQ ID NO: 6) | (SEQ ID NO: 66) | (SEQ ID NO: 140) | |
| | SSVPPY | YTSAATLV | AVSEMNYGGSQGNLI | 213 |
| | (SEQ ID NO: 9) | (SEQ ID NO: 69) | (SEQ ID NO: 141) | |
| | DSVNN | IPSGT | AVEGYKLS | 208 |
| | (SEQ ID NO: 6) | (SEQ ID NO: 66) | (SEQ ID NO: 142) | |
| | VSPFSN | MTFSENT | VAMNRDDKII | 158 |
| | (SEQ ID NO: 10) | (SEQ ID NO: 70) | (SEQ ID NO: 143) | |
| | DSVNN | IPSGT | AVENYGQNFV | 120 |
| | (SEQ ID NO: 6) | (SEQ ID NO: 66) | (SEQ ID NO: 144) | |
| | TSINN | IRSNERE | ATVSTSGTYKYI | 109 |
| | (SEQ ID NO: 11) | (SEQ ID NO: 71) | (SEQ ID NO: 145) | |
| | SSVSVY | YLSGSTLV | AVSDTGFQKLV | 102 |
| | (SEQ ID NO: 7) | (SEQ ID NO: 67) | (SEQ ID NO: 146) | |
| | DSVNN | IPSGT | AVDYYGQNFV | 95 |
| | (SEQ ID NO: 6) | (SEQ ID NO: 66) | (SEQ ID NO: 147) | |
| | DSVNN | IPSGT | AVYNYGQNFV | 71 |
| | (SEQ ID NO: 6) | (SEQ ID NO: 66) | (SEQ ID NO: 148) | |
| | DSVNN | IPSGT | AGDNYGQNFV | 69 |
| | (SEQ ID NO: 6) | (SEQ ID NO: 66) | (SEQ ID NO: 149) | |
| | DSVNN | IPSGT | AVDTYGQNFV | 52 |
| | (SEQ ID NO: 6) | (SEQ ID NO: 66) | (SEQ ID NO: 150) | |
| | DSVNN | IPSGT | AVANYGQNFV | 50 |
| | (SEQ ID NO: 6) | (SEQ ID NO: 66) | (SEQ ID NO: 151) | |
| | SSNFYA | MTLNGDE | AFMSGYSTLT | 41 |
| | (SEQ ID NO: 12) | (SEQ ID NO: 72) | (SEQ ID NO: 152) | |
| | DRGSQS | IYSNGD | AVNLGGGGADGLT | 41 |
| | (SEQ ID NO: 13) | (SEQ ID NO: 73) | (SEQ ID NO: 153) | |
| | DSVNN | IPSGT | AVEGYSGAGSYQLT | 39 |
| | (SEQ ID NO: 6) | (SEQ ID NO: 66) | (SEQ ID NO: 154) | |
| | DSVNN | IPSGT | AVDHYGQNFV | 35 |
| | (SEQ ID NO: 6) | (SEQ ID NO: 66) | (SEQ ID NO: 155) | |
| | DSVNN | IPSGT | AVEPHNARLM | 33 |
| | (SEQ ID NO: 6) | (SEQ ID NO: 66) | (SEQ ID NO: 156) | |
| | DSVNN | IPSGT | AVDKYGQNFV | 27 |
| | (SEQ ID NO: 6) | (SEQ ID NO: 66) | (SEQ ID NO: 157) | |
| | TSESDYY | QEAYKQQN | AYRSAVTGNQFY | 23 |
| | (SEQ ID NO: 14) | (SEQ ID NO: 74) | (SEQ ID NO: 158) | |
| | DSVNN | IPSGT | AVHNYGQNFV | 21 |
| | (SEQ ID NO: 6) | (SEQ ID NO: 66) | (SEQ ID NO: 159) | |
| | TSGFNG | NVLDGL | AVDLNSGYSTLT | 17 |
| | (SEQ ID NO: 15) | (SEQ ID NO: 75) | (SEQ ID NO: 160) | |
| | SSVSVY | YLSGSTLV | AVSDPGDEKLT | 15 |
| | (SEQ ID NO: 7) | (SEQ ID NO: 67) | (SEQ ID NO: 161) | |
| | TSINN | IRSNERE | ATVQNTGTASKLT | 13 |
| | (SEQ ID NO: 11) | (SEQ ID NO: 71) | (SEQ ID NO: 162) | |
| | NYSPAY | IRENEKE | ALGTEMTRS | 11 |
| | (SEQ ID NO: 16) | (SEQ ID NO: 76) | (SEQ ID NO: 163) | |
| | TRDTTYY | RNSFDEQN | ALSDSEGAQKLV | 10 |
| | (SEQ ID NO: 17) | (SEQ ID NO: 77) | (SEQ ID NO: 164) | |
| | DRGSQS | IYSNGD | AVDGQKLL | 10 |
| | (SEQ ID NO: 13) | (SEQ ID NO: 73) | (SEQ ID NO: 165) | |
| | TSDPSYG | QGSYDQQN | AMREGGDDKII | 9 |
| | (SEQ ID NO: 18) | (SEQ ID NO: 82) | (SEQ ID NO: 166) | |
| | TISGNEY | GLKNN | IVRVASGGGADGLT | 8 |
| | (SEQ ID NO: 8) | (SEQ ID NO: 68) | (SEQ ID NO: 167) | |
| | NYSPAY | IRENEKE | ALRRLQNY | 8 |
| | (SEQ ID NO: 16) | (SEQ ID NO: 76) | (SEQ ID NO: 168) | |
| | DSVNN | IPSGT | AVLPQGGSEKLV | 8 |
| | (SEQ ID NO: 6) | (SEQ ID NO: 66) | (SEQ ID NO: 169) | |
| | DSVNN | IPSGT | AVDNRGQNFV | 8 |
| | (SEQ ID NO: 6) | (SEQ ID NO: 66) | (SEQ ID NO: 170) | |
| | TSGFYG | DALDGL | ALYNFNKFY | 7 |
| | (SEQ ID NO: 19) | (SEQ ID NO: 83) | (SEQ ID NO: 171) | |
| | SIFNT | LYKAGEL | AGQLTLATQAN* | 7 |
| | (SEQ ID NO: 20) | (SEQ ID NO: 84) | (SEQ ID NO: 172) | |
| | DSVNN | IPSGT | AVDSCGQNFV | 7 |
| | (SEQ ID NO: 6) | (SEQ ID NO: 66) | (SEQ ID NO: 173) | |
| | DSVNN | IPSGT | AGITMVRIL | 7 |
| | (SEQ ID NO: 6) | (SEQ ID NO: 66) | (SEQ ID NO: 174) | |
| | YSGSPE | HISR | AQGSLALATQAN* | 6 |
| | (SEQ ID NO: 21) | (SEQ ID NO: 95) | (SEQ ID NO: 175) | |
| | TSESDYY | QEAYKQQN | ACFNSNSGYALN | 6 |
| | (SEQ ID NO: 14) | (SEQ ID NO: 74) | (SEQ ID NO: 176) | |
| | TSDPSYG | QGSYDQQN | AMRASGGYQKVT | 6 |
| | (SEQ ID NO: 18) | (SEQ ID NO: 82) | (SEQ ID NO: 177) | |
| | SSYSPS | YTSAATLV | VVSRIMEEAKEIS | 6 |
| | (SEQ ID NO: 22) | (SEQ ID NO: 69) | (SEQ ID NO: 178) | |
| | SSVPPY | YTTGATLV | AVSGYNNDMR | 6 |
| | (SEQ ID NO: 9) | (SEQ ID NO: 96) | (SEQ ID NO: 179) | |
| | NSAFQY | TYSSGN | AVGTGANNLF | 6 |
| | (SEQ ID NO: 23) | (SEQ ID NO: 97) | (SEQ ID NO: 180) | |
| | DSVNN | IPSGT | AG*LWSEFC | 6 |
| | (SEQ ID NO: 6) | (SEQ ID NO: 66) | (SEQ ID NO: 181) | |
| | DSASNY | IRSNVGE | AASIMTC | 6 |
| | (SEQ ID NO: 24) | (SEQ ID NO: 80) | (SEQ ID NO: 182) | |
| | TSDPSYG | QGSYDQQN | AMDVYNQGGKLI | 5 |
| | (SEQ ID NO: 18) | (SEQ ID NO: 82) | (SEQ ID NO: 183) | |
| | SSYSPS | YTSAATLV | VVSGVGQNFV | 5 |
| | (SEQ ID NO: 22) | (SEQ ID NO: 69) | (SEQ ID NO: 184) | |
| | DSVNN | IPSGT | AVDNCGQNFV | 5 |
| | (SEQ ID NO: 6) | (SEQ ID NO: 66) | (SEQ ID NO: 185) | |
| | DSVNN | IPSGT | AVDDHGQNFV | 5 |
| | (SEQ ID NO: 6) | (SEQ ID NO: 66) | (SEQ ID NO: 186) | |
| | DSVNN | IPSGT | AMDNYGQNFV | 5 |
| | (SEQ ID NO: 6) | (SEQ ID NO: 66) | (SEQ ID NO: 187) | |
| | SSVSVY | YLSGSTLV | AVTFAGGGNKLT | 4 |
| | (SEQ ID NO: 7) | (SEQ ID NO: 67) | (SEQ ID NO: 188) | |
| | SSVSVY | YLSGSTLV | AVAFTGGGNKLT | 4 |
| | (SEQ ID NO: 7) | (SEQ ID NO: 67) | (SEQ ID NO: 189) | |
| | SSVPPY | YTSAATLV | AVSLNDYKLS | 4 |
| | (SEQ ID NO: 9) | (SEQ ID NO: 69) | (SEQ ID NO: 190) | |
| | NSASDY | IRSNMDK | AEISYSSASKII | 4 |
| | (SEQ ID NO: 25) | (SEQ ID NO: 98) | (SEQ ID NO: 191) | |
| | DSVNN | IPSGT | TVDNYGQNFV | 4 |
| | (SEQ ID NO: 6) | (SEQ ID NO: 66) | (SEQ ID NO: 192) | |
| | DSVNN | IPSGT | AVDNYSQNFV | 4 |
| | (SEQ ID NO: 6) | (SEQ ID NO: 66) | (SEQ ID NO: 193) | |
| | DSVNN | IPSGT | AVDNHGQNFV | 4 |
| | (SEQ ID NO: 6) | (SEQ ID NO: 66) | (SEQ ID NO: 194) | |
| | TSESDYY | QEAYKQQN | AYRSHDMR | 3 |
| | (SEQ ID NO: 14) | (SEQ ID NO: 74) | (SEQ ID NO: 195) | |
| | TSESDYY | QEAYKQQN | AYGGGSEKLV | 3 |
| | (SEQ ID NO: 14) | (SEQ ID NO: 74) | (SEQ ID NO: 196) | |
| | SSVSVY | YLSGSTLV | AVTSTGGGNKLT | 3 |
| | (SEQ ID NO: 7) | (SEQ ID NO: 67) | (SEQ ID NO: 197) | |
| | SSVSVY | YLSGSTLV | AVTLTGGGNKLT | 3 |
| | (SEQ ID NO: 7) | (SEQ ID NO: 67) | (SEQ ID NO: 198) | |
| | SSVSVY | YLSGSTLV | AVSEMNYGGSQGNLI | 3 |
| | (SEQ ID NO: 7) | (SEQ ID NO: 67) | (SEQ ID NO: 141) | |
| | NYSPAY | IRENEKE | APPSGSARQLT | 3 |
| | (SEQ ID NO: 16) | (SEQ ID NO: 76) | (SEQ ID NO: 199) | |
| | DSVNN | IPSGT | AVNSYGQNFV | 3 |
| | (SEQ ID NO: 6) | (SEQ ID NO: 66) | (SEQ ID NO: 200) | |
| | DSVNN | IPSGT | AVNDYGQNFV | 3 |
| | (SEQ ID NO: 6) | (SEQ ID NO: 66) | (SEQ ID NO: 201) | |
| | DSVNN | IPSGT | AAVNYGQNFV | 3 |
| | (SEQ ID NO: 6) | (SEQ ID NO: 66) | (SEQ ID NO: 202) | |
| | DSVNN | IPSGT | AANNYGQNFV | 3 |
| | (SEQ ID NO: 6) | (SEQ ID NO: 66) | (SEQ ID NO: 203) | |
| | YGGTVN | YFSGDPLV | AVNRNTGNQFY | 2 |
| | (SEQ ID NO: 26) | (SEQ ID NO: 99) | (SEQ ID NO: 204) | |
| | VSPFSN | MTFSENT | VVSAKEAKEIS | 2 |
| | (SEQ ID NO: 10) | (SEQ ID NO: 70) | (SEQ ID NO: 205) | |
| | VSPFSN | MTFSENT | VVSAEGRQRLNPGEAI | 2 |
| | (SEQ ID NO: 10) | (SEQ ID NO: 70) | (SEQ ID NO: 206) | |
| | TSINN | IRSNERE | ATGSNDYKLS | 2 |
| | (SEQ ID NO: 11) | (SEQ ID NO: 71) | (SEQ ID NO: 207) | |
| | TSINN | IRSNERE | ATDGRGSYIPT | 2 |
| | (SEQ ID NO: 11) | (SEQ ID NO: 71) | (SEQ ID NO: 208) | |
| | TSINN | IRSNERE | ATDEDSSYKLI | 2 |
| | (SEQ ID NO: 11) | (SEQ ID NO: 71) | (SEQ ID NO: 209) | |
| | TSGFNG | NVLDGL | AVSDSNYQLI | 2 |
| | (SEQ ID NO: 15) | (SEQ ID NO: 75) | (SEQ ID NO: 210) | |
| | TSESDYY | QEAYKQQN | AYRSAGGATNKLI | 2 |
| | (SEQ ID NO: 14) | (SEQ ID NO: 74) | (SEQ ID NO: 211) | |
| | TSENNYY | QEAYKQQN | AFMKHSGVNDMR | 2 |
| | (SEQ ID NO: 31) | (SEQ ID NO: 74) | (SEQ ID NO: 212) | |
| | TISGNEY | GLKNN | IVSWGSNSGYALN | 2 |
| | (SEQ ID NO: 8) | (SEQ ID NO: 68) | (SEQ ID NO: 213) | |
| | TISGNEY | GLKNN | ICSGNTPLV | 2 |
| | (SEQ ID NO: 8) | (SEQ ID NO: 68) | (SEQ ID NO: 214) | |
| | TISGNEY | GLKNN | IANWGSNSGYALN | 2 |
| | (SEQ ID NO: 8) | (SEQ ID NO: 68) | (SEQ ID NO: 215) | |
| | SSVSVY | YLSGSTLV | AVTYTGGGNKLT | 2 |
| | (SEQ ID NO: 7) | (SEQ ID NO: 67) | (SEQ ID NO: 216) | |
| | SSVSVY | YLSGSTLV | AVTFMGGGNKLT | 2 |
| | (SEQ ID NO: 7) | (SEQ ID NO: 67) | (SEQ ID NO: 217) | |
| | SSVSVY | YLSGSTLV | AVTFKGGGNKLT | 2 |
| | (SEQ ID NO: 7) | (SEQ ID NO: 67) | (SEQ ID NO: 218) | |
| | SSVSVY | YLSGSTLV | AVSDRGETSW | 2 |
| | (SEQ ID NO: 7) | (SEQ ID NO: 67) | (SEQ ID NO: 219) | |
| | SSVSVY | YLSGSTLV | AVSDAGFQKLV | 2 |
| | (SEQ ID NO: 7) | (SEQ ID NO: 67) | (SEQ ID NO: 220) | |
| | SSVSVY | YLSGSTLV | AATFTGGGNKLT | 2 |
| | (SEQ ID NO: 7) | (SEQ ID NO: 67) | (SEQ ID NO: 221) | |
| | SSVPPY | YTSAATLV | AVSGMNYGGSQGNLI | 2 |
| | (SEQ ID NO: 9) | (SEQ ID NO: 69) | (SEQ ID NO: 222) | |
| | NYSPAY | IRENEKE | APYTGRRALT | 2 |
| | (SEQ ID NO: 16) | (SEQ ID NO: 76) | (SEQ ID NO: 223) | |
| | DSVNN | IPSGT | VVDNYGQNFV | 2 |
| | (SEQ ID NO: 6) | (SEQ ID NO: 66) | (SEQ ID NO: 224) | |
| | DSVNN | IPSGT | VADNYGQNFV | 2 |
| | (SEQ ID NO: 6) | (SEQ ID NO: 66) | (SEQ ID NO: 225) | |
| | DSVNN | IPSGT | TADNYGQNFV | 2 |
| | (SEQ ID NO: 6) | (SEQ ID NO: 66) | (SEQ ID NO: 226) | |
| | DSVNN | IPSGT | CG*LWSEFC | 2 |
| | (SEQ ID NO: 6) | (SEQ ID NO: 66) | (SEQ ID NO: 227) | |
| | DSVNN | IPSGT | AWITMVRIL | 2 |
| | (SEQ ID NO: 6) | (SEQ ID NO: 66) | (SEQ ID NO: 228) | |
| | DSVNN | IPSGT | AVSNYGQNFV | 2 |
| | (SEQ ID NO: 6) | (SEQ ID NO: 66) | (SEQ ID NO: 229) | |
| | DSVNN | IPSGT | AVSNDYKLS | 2 |
| | (SEQ ID NO: 6) | (SEQ ID NO: 66) | (SEQ ID NO: 230) | |
| | DSVNN | IPSGT | AVDS*GQNFV | 2 |
| | (SEQ ID NO: 6) | (SEQ ID NO: 66) | (SEQ ID NO: 231) | |
| | DSVNN | IPSGT | AVDNYVRIL | 2 |
| | (SEQ ID NO: 6) | (SEQ ID NO: 66) | (SEQ ID NO: 232) | |
| | DSVNN | IPSGT | AVDNHSQNFV | 2 |
| | (SEQ ID NO: 6) | (SEQ ID NO: 66) | (SEQ ID NO: 233) | |
| | DRVSQS | IYSNGD | AVFGSNTGKLI | 2 |
| | (SEQ ID NO: 32) | (SEQ ID NO: 73) | (SEQ ID NO: 234) | |
| | ATGYPS | ATKADDK | ALRSNDYKLS | 2 |
| | (SEQ ID NO: 33) | (SEQ ID NO: 100) | (SEQ ID NO: 235) | |
| | | | AVRIAFWGLPESY | 2 |
| | | | (SEQ ID NO: 236) | |
| TCRbeta | SGHNT | YYREEE | ASSLAGYEQY | 570775 |
| Donor Y | (SEQ ID NO: 34) | (SEQ ID NO: 101) | (SEQ ID NO: 237) | |
| (CS9.3) | | | | |
| | SGHDT | YYEEEE | ASSLGQGKH*SF | 21232 |
| | (SEQ ID NO: 35) | (SEQ ID NO: 102) | (SEQ ID NO: 238) | |
| | SQVTM | ANQGSEA | SVEGGSSGANVLT | 15824 |
| | (SEQ ID NO: 36) | (SEQ ID NO: 90) | (SEQ ID NO: 239) | |
| | SGHNT | YYREEE | ASSSAGYEQY | 1390 |
| | (SEQ ID NO: 34) | (SEQ ID NO: 101) | (SEQ ID NO: 240) | |
| | SGHNT | YYREEE | ASSLAGCEQY | 1191 |
| | (SEQ ID NO: 34) | (SEQ ID NO: 101) | (SEQ ID NO: 241) | |
| | SGHDY | FNNNVP | ASTSWGVSYNEQF | 1176 |
| | (SEQ ID NO: 37) | (SEQ ID NO: 103) | (SEQ ID NO: 242) | |
| | SGHNT | YYREEE | ASSLASYEQY | 1036 |
| | (SEQ ID NO: 34) | (SEQ ID NO: 101) | (SEQ ID NO: 243) | |
| | SGHNT | YYREEE | ASSLAGHEQY | 1003 |
| | (SEQ ID NO: 34) | (SEQ ID NO: 101) | (SEQ ID NO: 244) | |
| | KGHSH | LQKENI | ASSPPEGFGNEQF | 612 |
| | (SEQ ID NO: 38) | (SEQ ID NO: 81) | (SEQ ID NO: 245) | |
| | SGHNT | YYREEE | ASSLTGYEQY | 546 |
| | (SEQ ID NO: 34) | (SEQ ID NO: 101) | (SEQ ID NO: 246) | |
| | DFQATT | SNEGSKA | SANLAKSSYNEQF | 265 |
| | (SEQ ID NO: 39) | (SEQ ID NO: 89) | (SEQ ID NO: 247) | |
| | DFQATT | SNEGSKA | SAPRDPDADTQY | 231 |
| | (SEQ ID NO: 39) | (SEQ ID NO: 89) | (SEQ ID NO: 248) | |
| | GTSNPN | SVGIG | AWDRTGEVEQY | 186 |
| | (SEQ ID NO: 40) | (SEQ ID NO: 104) | (SEQ ID NO: 249) | |
| | SGHNT | YYREEE | ASS*AGYEQY | 174 |
| | (SEQ ID NO: 34) | (SEQ ID NO: 101) | (SEQ ID NO: 250) | |
| | SGHNT | YYREEE | AAAWPATSS | 151 |
| | (SEQ ID NO: 34) | (SEQ ID NO: 101) | (SEQ ID NO: 251) | |
| | SGHNT | YYREEE | ASSMAGYEQY | 140 |
| | (SEQ ID NO: 34) | (SEQ ID NO: 101) | (SEQ ID NO: 252) | |
| | GTSNPN | SVGIG | AWSFHPGLAAYNEQF | 108 |
| | (SEQ ID NO: 40) | (SEQ ID NO: 104) | (SEQ ID NO: 253) | |
| | SGHDN | FVKESK | ASSQLRGGSPLH | 100 |
| | (SEQ ID NO: 41) | (SEQ ID NO: 105) | (SEQ ID NO: 254) | |
| | MNHEY | SVGAGI | ASSGQGGSNTEAF | 98 |
| | (SEQ ID NO: 42) | (SEQ ID NO: 88) | (SEQ ID NO: 255) | |
| | SGHDT | YYEEEE | ASSLGQGRH*SF | 89 |
| | (SEQ ID NO: 35) | (SEQ ID NO: 102) | (SEQ ID NO: 256) | |
| | SGHVS | FNYEAQ | ASSLAEDTQY | 81 |
| | (SEQ ID NO: 43) | (SEQ ID NO: 106) | (SEQ ID NO: 257) | |
| | SGHNT | YYREEE | ASSWAGYEQY | 70 |
| | (SEQ ID NO: 34) | (SEQ ID NO: 101) | (SEQ ID NO: 258) | |
| | SGHDT | YYEEEE | ASSLGRGKH*SF | 70 |
| | (SEQ ID NO: 35) | (SEQ ID NO: 102) | (SEQ ID NO: 259) | |
| | SGHNT | YYREEE | ASSLAGFEQY | 68 |
| | (SEQ ID NO: 34) | (SEQ ID NO: 101) | (SEQ ID NO: 260) | |
| | SGHNT | YYREEE | ASSLAG*EQY | 59 |
| | (SEQ ID NO: 34) | (SEQ ID NO: 101) | (SEQ ID NO: 261) | |
| | SGHDT | YYEEEE | ASSLGQGKR*SF | 58 |
| | (SEQ ID NO: 35) | (SEQ ID NO: 102) | (SEQ ID NO: 262) | |
| | SGHDT | YYEEEE | ASSLGQGEH*SF | 57 |
| | (SEQ ID NO: 35) | (SEQ ID NO: 102) | (SEQ ID NO: 263) | |
| | GTSNPN | SVGIG | AYSTGYFGYT | 54 |
| | (SEQ ID NO: 40) | (SEQ ID NO: 104) | (SEQ ID NO: 264) | |
| | SQVTM | ANQGSEA | SVGGGSSGANVLT | 49 |
| | (SEQ ID NO: 36) | (SEQ ID NO: 90) | (SEQ ID NO: 265) | |
| | SGHNT | YYREEE | ASSVAGYEQY | 49 |
| | (SEQ ID NO: 34) | (SEQ ID NO: 101) | (SEQ ID NO: 266) | |
| | SQVTM | ANQGSEA | SVEGGSPGANVLT | 47 |
| | (SEQ ID NO: 36) | (SEQ ID NO: 90) | (SEQ ID NO: 267) | |
| | SGHDT | YYEEEE | ASSSGQGKH*SF | 47 |
| | (SEQ ID NO: 35) | (SEQ ID NO: 102) | (SEQ ID NO: 268) | |
| | SNHLY | FYNNEI | ASSESRYGRDTDTQY | 39 |
| | (SEQ ID NO: 44) | (SEQ ID NO: 94) | (SEQ ID NO: 269) | |
| | MNHEY | SVGEGT | ASSYSYSTGPELNTEAF | 38 |
| | (SEQ ID NO: 42) | (SEQ ID NO: 91) | (SEQ ID NO: 270) | |
| | SQVTM | ANQGSEA | SVEGGPSGANVLT | 36 |
| | (SEQ ID NO: 36) | (SEQ ID NO: 90) | (SEQ ID NO: 271) | |
| | SGHNT | YYREEE | ASSLAGNEQY | 34 |
| | (SEQ ID NO: 34) | (SEQ ID NO: 101) | (SEQ ID NO: 272) | |
| | SGHNT | YYREEE | ASSLAAYEQY | 33 |
| | (SEQ ID NO: 34) | (SEQ ID NO: 101) | (SEQ ID NO: 273) | |
| | SGHNT | YYREEE | ASSLSFDSEQY | 32 |
| | (SEQ ID NO: 34) | (SEQ ID NO: 101) | (SEQ ID NO: 274) | |
| | SGHDT | YYEEEE | ASSLSQGKH*SF | 32 |
| | (SEQ ID NO: 35) | (SEQ ID NO: 102) | (SEQ ID NO: 275) | |
| | SGHNT | YYREEE | ASSFAGYEQY | 31 |
| | (SEQ ID NO: 34) | (SEQ ID NO: 101) | (SEQ ID NO: 276) | |
| | SQVTM | ANQGSEA | SARQGLTEAF | 27 |
| | (SEQ ID NO: 36) | (SEQ ID NO: 90) | (SEQ ID NO: 277) | |
| | GTSNPN | SVGIG | AWSVLYGTEY | 27 |
| | (SEQ ID NO: 40) | (SEQ ID NO: 104) | (SEQ ID NO: 278) | |
| | SGHNT | YYREEE | ASSLAGSEQY | 26 |
| | (SEQ ID NO: 34) | (SEQ ID NO: 101) | (SEQ ID NO: 279) | |
| | SQVTM | ANQGSEA | SVEGDPLGPTS* | 24 |
| | (SEQ ID NO: 36) | (SEQ ID NO: 90) | (SEQ ID NO: 280) | |
| | SGHNT | YYREEE | ASSLSGYEQY | 24 |
| | (SEQ ID NO: 34) | (SEQ ID NO: 101) | (SEQ ID NO: 281) | |
| | SQVTM | ANQGSEA | SVEEGSSGANVLT | 22 |
| | (SEQ ID NO: 36) | (SEQ ID NO: 90) | (SEQ ID NO: 282) | |
| | SGHNT | YYREEE | ASSLPGYEQY | 21 |
| | (SEQ ID NO: 34) | (SEQ ID NO: 101) | (SEQ ID NO: 283) | |
| | LNHNV | YYDKDF | ATSREGTGENIQY | 21 |
| | (SEQ ID NO: 45) | (SEQ ID NO: 107) | (SEQ ID NO: 284) | |
| | SGHDT | YYEEEE | ASSFSIRASYEQY | 19 |
| | (SEQ ID NO: 35) | (SEQ ID NO: 102) | (SEQ ID NO: 285) | |
| | SGHNT | YYREEE | ASSLAGDEQY | 17 |
| | (SEQ ID NO: 34) | (SEQ ID NO: 101) | (SEQ ID NO: 286) | |
| | | | ASSVASTGELF | 16 |
| | | | (SEQ ID NO: 287) | |
| | SGHDT | YYEEEE | ASSLGRGNTEAF | 15 |
| | (SEQ ID NO: 35) | (SEQ ID NO: 102) | (SEQ ID NO: 288) | |
| | SGHAT | FQNNGV | ASSPIRREGEQY | 15 |
| | (SEQ ID NO: 46) | (SEQ ID NO: 108) | (SEQ ID NO: 289) | |
| | KGHSH | LQKENI | ASFVYSAGDSYNEQF | 15 |
| | (SEQ ID NO: 38) | (SEQ ID NO: 81) | (SEQ ID NO: 290) | |
| | DFQATT | SNEGSKA | SARNRVYEQY | 15 |
| | (SEQ ID NO: 39) | (SEQ ID NO: 89) | (SEQ ID NO: 291) | |
| | SGHDT | YYEEEE | ASSLGQGNTEAF | 14 |
| | (SEQ ID NO: 35) | (SEQ ID NO: 102) | (SEQ ID NO: 292) | |
| | MNHEY | SVGAGI | ASSPPGENEQY | 12 |
| | (SEQ ID NO: 42) | (SEQ ID NO: 88) | (SEQ ID NO: 293) | |
| | LNHDA | SQIVND | ASTDTDLGEQY | 12 |
| | (SEQ ID NO: 47) | (SEQ ID NO: 109) | (SEQ ID NO: 294) | |
| | SQVTM | ANQGSEA | SVERGSSGANVLT | 11 |
| | (SEQ ID NO: 36) | (SEQ ID NO: 90) | (SEQ ID NO: 295) | |
| | SGHNT | YYREEE | ASSLGQGKH*SF | 11 |
| | (SEQ ID NO: 34) | (SEQ ID NO: 101) | (SEQ ID NO: 238) | |
| | SGHDT | YYEEEE | ASSLGQGNH*SF | 11 |
| | (SEQ ID NO: 35) | (SEQ ID NO: 102) | (SEQ ID NO: 296) | |
| | SGHDT | YYEEEE | ASSLARGNTEAF | 11 |
| | (SEQ ID NO: 35) | (SEQ ID NO: 102) | (SEQ ID NO: 297) | |
| | SGHDT | YYEEEE | AAAWARGNTEAF | 11 |
| | (SEQ ID NO: 35) | (SEQ ID NO: 102) | (SEQ ID NO: 298) | |
| | ENHRY | SYGVKD | ALSDSGTIYEQY | 11 |
| | (SEQ ID NO: 48) | (SEQ ID NO: 110) | (SEQ ID NO: 299) | |
| | WSHSY | SAAADI | ASSVPLEGGSGPQDTQY | 10 |
| | (SEQ ID NO: 49) | (SEQ ID NO: 111) | (SEQ ID NO: 300) | |
| | SGHDT | YYEEEE | ASSLGQGKY*SF | 10 |
| | (SEQ ID NO: 35) | (SEQ ID NO: 102) | (SEQ ID NO: 301) | |
| | LGHNT | FRNRAP | ASGLYNRGNEQF | 10 |
| | (SEQ ID NO: 50) | (SEQ ID NO: 112) | (SEQ ID NO: 302) | |
| | SQVTM | ANQGSEA | SVEGGSSGPTS* | 9 |
| | (SEQ ID NO: 36) | (SEQ ID NO: 90) | (SEQ ID NO: 303) | |
| | SGHNT | YYREEE | ASGLAGYEQY | 9 |
| | (SEQ ID NO: 34) | (SEQ ID NO: 101) | (SEQ ID NO: 304) | |
| | KGHSH | LQKENI | ASSRTRYTDTQY | 9 |
| | (SEQ ID NO: 38) | (SEQ ID NO: 81) | (SEQ ID NO: 305) | |
| | SGHNT | YYREEE | ASNLAGYEQY | 8 |
| | (SEQ ID NO: 34) | (SEQ ID NO: 101) | (SEQ ID NO: 306) | |
| | SGHDY | FNNNVP | ASASWGVSYNEQF | 8 |
| | (SEQ ID NO: 37) | (SEQ ID NO: 103) | (SEQ ID NO: 307) | |
| | SGHDT | YYEEEE | ASSLGQGETLKL | 8 |
| | (SEQ ID NO: 35) | (SEQ ID NO: 102) | (SEQ ID NO: 308) | |
| | SQVTM | ANQGSEA | SVVGGSSGANVLT | 7 |
| | (SEQ ID NO: 36) | (SEQ ID NO: 90) | (SEQ ID NO: 309) | |
| | SQVTM | ANQGSEA | SVGANVAGGKETQY | 7 |
| | (SEQ ID NO: 36) | (SEQ ID NO: 90) | (SEQ ID NO: 310) | |
| | | | ASSVTGTVNTEAF | 7 |
| | | | (SEQ ID NO: 311) | |
| | SQVTM | ANQGSEA | SVKGGSSGANVLT | 6 |
| | (SEQ ID NO: 36) | (SEQ ID NO: 90) | (SEQ ID NO: 312) | |
| | SQVTM | ANQGSEA | SVEGGSTGANVLT | 6 |
| | (SEQ ID NO: 36) | (SEQ ID NO: 90) | (SEQ ID NO: 313) | |
| | SGHDT | YYGEEE | ASSLGQGIH*SF | 6 |
| | (SEQ ID NO: 35) | (SEQ ID NO: 113) | (SEQ ID NO: 314) | |
| | SGHDT | YYEEEE | ASSLGQGKL*SF | 6 |
| | (SEQ ID NO: 35) | (SEQ ID NO: 102) | (SEQ ID NO: 315) | |
| | SGHDN | FVKESK | ASSQDIEV*EAF | 6 |
| | (SEQ ID NO: 41) | (SEQ ID NO: 105) | (SEQ ID NO: 316) | |
| | LGHDT | YNNKEL | ASSLRLNTEAF | 6 |
| | (SEQ ID NO: 51) | (SEQ ID NO: 114) | (SEQ ID NO: 317) | |
| | | | ASSVEAGVSGNTIY | 6 |
| | | | (SEQ ID NO: 318) | |
| | SQVTM | ANQGSEA | SVVRQGHYEAF | 5 |
| | (SEQ ID NO: 36) | (SEQ ID NO: 90) | (SEQ ID NO: 319) | |
| | SQVTM | ANQGSEA | SVEGGSFGANVLT | 5 |
| | (SEQ ID NO: 36) | (SEQ ID NO: 90) | (SEQ ID NO: 320) | |
| | SNHLY | FYNNEI | ASSPGRILTDTQY | 5 |
| | (SEQ ID NO: 44) | (SEQ ID NO: 94) | (SEQ ID NO: 321) | |
| | SGHNT | YYREEE | ASSLADYEQY | 5 |
| | (SEQ ID NO: 34) | (SEQ ID NO: 101) | (SEQ ID NO: 322) | |
| | MNHEY | SVGAGI | ASSGGLNQPQH | 5 |
| | (SEQ ID NO: 42) | (SEQ ID NO: 88) | (SEQ ID NO: 323) | |
| | MDHEN | SYDVKM | ASKVQGSEDTQY | 5 |
| | (SEQ ID NO: 52) | (SEQ ID NO: 93) | (SEQ ID NO: 324) | |
| | KGHSH | LQKENI | ASSPPGGFGNEQF | 5 |
| | (SEQ ID NO: 38) | (SEQ ID NO: 81) | (SEQ ID NO: 325) | |
| | SNHLY | FYNNEI | ASSGAGQGSSYEQY | 4 |
| | (SEQ ID NO: 44) | (SEQ ID NO: 94) | (SEQ ID NO: 326) | |
| | SGHNT | YYREEE | ASSLGQGEH*SF | 4 |
| | (SEQ ID NO: 34) | (SEQ ID NO: 101) | (SEQ ID NO: 263) | |
| | SGHNT | YYREEE | ASSLASCEQY | 4 |
| | (SEQ ID NO: 34) | (SEQ ID NO: 101) | (SEQ ID NO: 327) | |
| | SGHNT | YYREEE | ASSLAGYRQY | 4 |
| | (SEQ ID NO: 34) | (SEQ ID NO: 101) | (SEQ ID NO: 328) | |
| | SGHNT | YYREEE | ASSLAGYKQY | 4 |
| | (SEQ ID NO: 34) | (SEQ ID NO: 101) | (SEQ ID NO: 329) | |
| | SGHDY | FNNNVP | ASTSWASPTMSS | 4 |
| | (SEQ ID NO: 37) | (SEQ ID NO: 103) | (SEQ ID NO: 330) | |
| | SGHDY | FNNNVP | ASTSRGVSYNEQF | 4 |
| | (SEQ ID NO: 37) | (SEQ ID NO: 103) | (SEQ ID NO: 331) | |
| | SGHDT | YYEEEE | ASSFGQGKH*SF | 4 |
| | (SEQ ID NO: 35) | (SEQ ID NO: 102) | (SEQ ID NO: 332) | |
| | SGHDT | YYEEEE | ASS*GQGKH*SF | 4 |
| | (SEQ ID NO: 35) | (SEQ ID NO: 102) | (SEQ ID NO: 333) | |
| | SGHNT | YYREEE | ASSLVGHEQY | 3 |
| | (SEQ ID NO: 34) | (SEQ ID NO: 101) | (SEQ ID NO: 334) | |
| | SGHNT | YYREEE | ASSLGRGKH*SF | 3 |
| | (SEQ ID NO: 34) | (SEQ ID NO: 101) | (SEQ ID NO: 259) | |
| | SGHNT | YYREEE | ASSLAGHGQY | 3 |
| | (SEQ ID NO: 34) | (SEQ ID NO: 101) | (SEQ ID NO: 335) | |
| | SGHNT | YYREEE | ASNSAGYEQY | 3 |
| | (SEQ ID NO: 34) | (SEQ ID NO: 101) | (SEQ ID NO: 336) | |
| | SGHNT | YYREEE | ASGSAGYEQY | 3 |
| | (SEQ ID NO: 34) | (SEQ ID NO: 101) | (SEQ ID NO: 337) | |
| | SGHDY | FNNNVP | ASTSWGISYNEQF | 3 |
| | (SEQ ID NO: 37) | (SEQ ID NO: 103) | (SEQ ID NO: 338) | |
| | SGHDY | FNNNVP | ASTSWGASYNEQF | 3 |
| | (SEQ ID NO: 37) | (SEQ ID NO: 103) | (SEQ ID NO: 339) | |
| | SGHDY | FNNNVP | ASTS*GVSYNEQF | 3 |
| | (SEQ ID NO: 37) | (SEQ ID NO: 103) | (SEQ ID NO: 340) | |
| | SGHDY | FNNNVP | AGTSWGVSYNEQF | 3 |
| | (SEQ ID NO: 37) | (SEQ ID NO: 103) | (SEQ ID NO: 341) | |
| | SGHDT | YYEEEE | ASSVGQGKH*SF | 3 |
| | (SEQ ID NO: 35) | (SEQ ID NO: 102) | (SEQ ID NO: 342) | |
| | SGHDT | YYEEEE | ASSMGQGKH*SF | 3 |
| | (SEQ ID NO: 35) | (SEQ ID NO: 102) | (SEQ ID NO: 343) | |
| | SGHDT | YYEEEE | ASSLCQGKH*SF | 3 |
| | (SEQ ID NO: 35) | (SEQ ID NO: 102) | (SEQ ID NO: 344) | |
| | MNHNS | SASEGT | ASRGLAGFNEQF | 3 |
| | (SEQ ID NO: 53) | (SEQ ID NO: 92) | (SEQ ID NO: 345) | |
| | MNHEY | SMNVEV | ASSLMRVGFRTDTQY | 3 |
| | (SEQ ID NO: 42) | (SEQ ID NO: 115) | (SEQ ID NO: 346) | |
| | MGHRA | YSYEKL | ASSQDELAGRTQY | 3 |
| | (SEQ ID NO: 54) | (SEQ ID NO: 116) | (SEQ ID NO: 347) | |
| | MDHEN | SYDVKM | ASTNSLTSTDTQY | 3 |
| | (SEQ ID NO: 52) | (SEQ ID NO: 93) | (SEQ ID NO: 348) | |
| | KGHSH | LQKENI | ASSPPEGLGNEQF | 3 |
| | (SEQ ID NO: 38) | (SEQ ID NO: 81) | (SEQ ID NO: 349) | |
| | ENHRY | SYGVKD | AISRADQETQY | 3 |
| | (SEQ ID NO: 48) | (SEQ ID NO: 110) | (SEQ ID NO: 350) | |
| | DFQATT | SNEGSKA | SARDRGATGELF | 3 |
| | (SEQ ID NO: 39) | (SEQ ID NO: 89) | (SEQ ID NO: 351) | |
| | SQVTM | ANQGSEA | SVEGGSAGANVLT | 2 |
| | (SEQ ID NO: 36) | (SEQ ID NO: 90) | (SEQ ID NO: 352) | |
| | SQVTM | ANQGSEA | SARQGRTEAF | 2 |
| | (SEQ ID NO: 36) | (SEQ ID NO: 90) | (SEQ ID NO: 353) | |
| | SGHYY | FNNNVP | ASTSWGVPYNEQF | 2 |
| | (SEQ ID NO: 55) | (SEQ ID NO: 103) | (SEQ ID NO: 354) | |
| | SGHNT | YYREEE | ASSLTSYEQY | 2 |
| | (SEQ ID NO: 34) | (SEQ ID NO: 101) | (SEQ ID NO: 355) | |
| | SGHNT | YYREEE | ASSLTGCEQY | 2 |
| | (SEQ ID NO: 34) | (SEQ ID NO: 101) | (SEQ ID NO: 356) | |
| | SGHNT | YYREEE | ASSLGQRKH*SF | 2 |
| | (SEQ ID NO: 34) | (SEQ ID NO: 101) | (SEQ ID NO: 357) | |
| | SGHNT | YYREEE | ASSLGQGRH*SF | 2 |
| | (SEQ ID NO: 34) | (SEQ ID NO: 101) | (SEQ ID NO: 256) | |
| | SGHNT | YYREEE | ASSLGQGKR*SF | 2 |
| | (SEQ ID NO: 34) | (SEQ ID NO: 101) | (SEQ ID NO: 262) | |
| | SGHNT | YYREEE | ASSLAGYK*Y | 2 |
| | (SEQ ID NO: 34) | (SEQ ID NO: 101) | (SEQ ID NO: 358) | |
| | SGHNT | YYREEE | ASGMAGYEQY | 2 |
| | (SEQ ID NO: 34) | (SEQ ID NO: 101) | (SEQ ID NO: 359) | |
| | SGHDT | YYEEEE | ASSWARGNTEAF | 2 |
| | (SEQ ID NO: 35) | (SEQ ID NO: 102) | (SEQ ID NO: 360) | |
| | SGHDT | YYEEEE | ASSLGQETLKL | 2 |
| | (SEQ ID NO: 35) | (SEQ ID NO: 102) | (SEQ ID NO: 361) | |
| | SEHNR | FQNEAQ | ASTLYEKLF | 2 |
| | (SEQ ID NO: 56) | (SEQ ID NO: 117) | (SEQ ID NO: 362) | |
| | PGHNT | YYREEE | ASDLAGYEQY | 2 |
| | (SEQ ID NO: 57) | (SEQ ID NO: 101) | (SEQ ID NO: 363) | |
| | MDHEN | SYDVKM | ASVGTGNVDEQY | 2 |
| | (SEQ ID NO: 52) | (SEQ ID NO: 93) | (SEQ ID NO: 364) | |
| | KGHSH | LQKENI | ASSPPEGSGNEQF | 2 |
| | (SEQ ID NO: 38) | (SEQ ID NO: 81) | (SEQ ID NO: 365) | |
| | KGHSH | LQKENI | ASSPPEGFSNEQF | 2 |
| | (SEQ ID NO: 38) | (SEQ ID NO: 81) | (SEQ ID NO: 366) | |
| | DFQATT | SNEGSKA | SANLARSSYNEQF | 2 |
| | (SEQ ID NO: 39) | (SEQ ID NO: 89) | (SEQ ID NO: 367) | |
| | DFQATT | SNEGSKA | SALDLAGSQETQY | 2 |
| | (SEQ ID NO: 39) | (SEQ ID NO: 89) | (SEQ ID NO: 368) | |
| TCRalpha | DSVNN | IPSGT | AVELFAAGNKLT | 66076 |
| Donor R | (SEQ ID NO: 6) | (SEQ ID NO: 66) | (SEQ ID NO: 369) | |
| (CS9.3) | | | | |
| | SVFSS | VVTGGEV | AGAVTGQLQQIL | 191 |
| | (SEQ ID NO: 58) | (SEQ ID NO: 118) | (SEQ ID NO: 370) | |
| | DSVNN | IPSGT | AVGLFAAGNKLT | 184 |
| | (SEQ ID NO: 6) | (SEQ ID NO: 66) | (SEQ ID NO: 371) | |
| | DSVNN | IPSGT | AVELLAAGNKLT | 161 |
| | (SEQ ID NO: 6) | (SEQ ID NO: 66) | (SEQ ID NO: 372) | |
| | DSVNN | IPSGT | AVELSAAGNKLT | 126 |
| | (SEQ ID NO: 6) | (SEQ ID NO: 66) | (SEQ ID NO: 373) | |
| | DSVNN | IPSGT | AVKLFAAGNKLT | 72 |
| | (SEQ ID NO: 6) | (SEQ ID NO: 66) | (SEQ ID NO: 374) | |
| | DSVNN | IPSGT | AVELFTAGNKLT | 65 |
| | (SEQ ID NO: 6) | (SEQ ID NO: 66) | (SEQ ID NO: 375) | |
| | DSVNN | IPSGT | AVELFATGNKLT | 58 |
| | (SEQ ID NO: 6) | (SEQ ID NO: 66) | (SEQ ID NO: 376) | |
| | DSVNN | IPSGT | AVELFVAGNKLT | 42 |
| | (SEQ ID NO: 6) | (SEQ ID NO: 66) | (SEQ ID NO: 377) | |
| | DSVNN | IPSGT | AVELFAAGNKL | |
| | (SEQ ID NO: 6) | (SEQ ID NO: 66) | (SEQ ID NO: 378) | |
| | DSVNN | IPSGT | AVVLFAAGNKLT | |
| | (SEQ ID NO: 6) | (SEQ ID NO: 66) | (SEQ ID NO: 379) | |
| | DSVNN | IPSGT | AVSYLLQATS* | |
| | (SEQ ID NO: 6) | (SEQ ID NO: 66) | (SEQ ID NO: 380) | |
| | DSVNN | IPSGT | AV*LFAAGNKLT | |
| | (SEQ ID NO: 6) | (SEQ ID NO: 66) | (SEQ ID NO: 381) | |
| | DSVNN | IPSGT | AVELFASGNKLT | |
| | (SEQ ID NO: 6) | (SEQ ID NO: 66) | (SEQ ID NO: 382) | |
| | DSVNN | IPSGT | AVELFAAATS* | |
| | (SEQ ID NO: 6) | (SEQ ID NO: 66) | (SEQ ID NO: 383) | |
| | DSVNN | IPSGT | AVDLFAAGNKLT | |
| | (SEQ ID NO: 6) | (SEQ ID NO: 66) | (SEQ ID NO: 384) | |
| | DSVNN | IPSGT | AVELFDAGNKLT | |
| | (SEQ ID NO: 6) | (SEQ ID NO: 66) | (SEQ ID NO: 385) | |
| | | IFSNGE | AASEGNYNVLY | |
| | | (SEQ ID NO: 85) | (SEQ ID NO: 386) | |
| | DSVNN | IPSGT | AVEVFAAGNKLT | |
| | (SEQ ID NO: 6) | (SEQ ID NO: 66) | (SEQ ID NO: 387) | |
| | DSVNN | IPSGT | AVELFSAGNKLT | |
| | (SEQ ID NO: 6) | (SEQ ID NO: 66) | (SEQ ID NO: 388) | |
| | DSVNN | IPSGT | AVALFAAGNKLT | |
| | (SEQ ID NO: 6) | (SEQ ID NO: 66) | (SEQ ID NO: 389) | |
| | DSVNN | IPSGT | AVEIFAAGNKLT | |
| | (SEQ ID NO: 6) | (SEQ ID NO: 66) | (SEQ ID NO: 390) | |
| | DSVNN | IPSGT | AVELFPAGNKLT | |
| | (SEQ ID NO: 6) | (SEQ ID NO: 66) | (SEQ ID NO: 391) | |
| | DSVNN | IPSGT | AVELFGAGNKLT | |
| | (SEQ ID NO: 6) | (SEQ ID NO: 66) | (SEQ ID NO: 392) | |
| | DSVNN | IPSGT | AVELFAEATS* | |
| | (SEQ ID NO: 6) | (SEQ ID NO: 66) | (SEQ ID NO: 393) | |
| TCRbeta | MDHEN | SYDVKM | ASSLISGSSYEQY | 111183 |
| Donor R | (SEQ ID NO: 52) | (SEQ ID NO: 93) | (SEQ ID NO: 394) | |
| (CS9.3) | | | | |
| | LNHDA | SQIVND | ASSIEGQK*TLKL | 7924 |
| | (SEQ ID NO: 47) | (SEQ ID NO: 109) | (SEQ ID NO: 395) | |
| | MDHEN | SYDVKM | ASSLISGSPYEQY | 368 |
| | (SEQ ID NO: 52) | (SEQ ID NO: 93) | (SEQ ID NO: 396) | |
| | MDHEN | SYDVKM | ASGLISGSSYEQY | 312 |
| | (SEQ ID NO: 52) | (SEQ ID NO: 93) | (SEQ ID NO: 397) | |
| | MDHEN | SYDVKM | ASSLIGGSSYEQY | 293 |
| | (SEQ ID NO: 52) | (SEQ ID NO: 93) | (SEQ ID NO: 398) | |
| | MDHEN | SYDVKM | ASSLVSGSSYEQY | 233 |
| | (SEQ ID NO: 52) | (SEQ ID NO: 93) | (SEQ ID NO: 399) | |
| | MDHEN | SYDVKM | ASSPISGSSYEQY | 230 |
| | (SEQ ID NO: 52) | (SEQ ID NO: 93) | (SEQ ID NO: 400) | |
| | MDHEN | SYDVKM | ASSLISGGSYEQY | 222 |
| | (SEQ ID NO: 52) | (SEQ ID NO: 93) | (SEQ ID NO: 401) | |
| | MDHEN | SYDVKM | ASNLISGSSYEQY | 98 |
| | (SEQ ID NO: 52) | (SEQ ID NO: 93) | (SEQ ID NO: 402) | |
| | MDHEN | SYDVKM | ASSLISGSFYEQY | 65 |
| | (SEQ ID NO: 52) | (SEQ ID NO: 93) | (SEQ ID NO: 403) | |
| | MDHEN | SYDVKM | ASSLISGSTYEQY | 45 |
| | (SEQ ID NO: 52) | (SEQ ID NO: 93) | (SEQ ID NO: 404) | |
| | LNHDA | SQIVND | ASSIEGQK*ALKL | 43 |
| | (SEQ ID NO: 47) | (SEQ ID NO: 109) | (SEQ ID NO: 405) | |
| | MDHEN | SYDVKM | ASRLISGSSYEQY | 38 |
| | (SEQ ID NO: 52) | (SEQ ID NO: 93) | (SEQ ID NO: 406) | |
| | LNHDA | SQIVND | ASSIEGQKWTLKL | 33 |
| | (SEQ ID NO: 47) | (SEQ ID NO: 109) | (SEQ ID NO: 407) | |
| | MDHEN | SYDVKM | AAV**VVAPTSS | 30 |
| | (SEQ ID NO: 52) | (SEQ ID NO: 93) | (SEQ ID NO: 408) | |
| | LNHDA | SQIVND | ASSIGGQK*TLKL | 27 |
| | (SEQ ID NO: 47) | (SEQ ID NO: 109) | (SEQ ID NO: 409) | |
| | LNHDA | SQIVND | ASSIEGQR*TLKL | 26 |
| | (SEQ ID NO: 47) | (SEQ ID NO: 109) | (SEQ ID NO: 410) | |
| | LNHDA | SQIVND | ASSIEGRK*TLKL | 23 |
| | (SEQ ID NO: 47) | (SEQ ID NO: 109) | (SEQ ID NO: 411) | |
| | LNHDA | SQIVND | ASSTEGQK*TLKL | 17 |
| | (SEQ ID NO: 47) | (SEQ ID NO: 109) | (SEQ ID NO: 412) | |
| | MDHEN | SYDVKM | ASSLISVAPTSS | 16 |
| | (SEQ ID NO: 52) | (SEQ ID NO: 93) | (SEQ ID NO: 413) | |
| | LNHDA | SQIVND | ASSIEGQE*TLKL | 16 |
| | (SEQ ID NO: 47) | (SEQ ID NO: 109) | (SEQ ID NO: 414) | |
| | MDHEN | SYDVKM | ARSLISGSSYEQY | 15 |
| | (SEQ ID NO: 52) | (SEQ ID NO: 93) | (SEQ ID NO: 415) | |
| | LNHDA | SQIVND | ASSMEGQK*TLKL | 15 |
| | (SEQ ID NO: 47) | (SEQ ID NO: 109) | (SEQ ID NO: 416) | |
| | LNHDA | SQIVND | ASSIEEQK*TLKL | 15 |
| | (SEQ ID NO: 47) | (SEQ ID NO: 109) | (SEQ ID NO: 417) | |
| | LNHDA | SQIVND | ASSIEGQKRTLKL | 14 |
| | (SEQ ID NO: 47) | (SEQ ID NO: 109) | (SEQ ID NO: 418) | |
| | MDHEN | SYDVKM | ASSLISGSAYEQY | 13 |
| | (SEQ ID NO: 52) | (SEQ ID NO: 93) | (SEQ ID NO: 419) | |
| | MDHEN | SYDVKM | ASCLISGSSYEQY | 9 |
| | (SEQ ID NO: 52) | (SEQ ID NO: 93) | (SEQ ID NO: 420) | |
| | MDHEN | SYDVKM | ASSLISGRSYEQY | 8 |
| | (SEQ ID NO: 52) | (SEQ ID NO: 93) | (SEQ ID NO: 421) | |
| | MDHEN | SYDVKM | ASSLISGSYYEQY | 6 |
| | (SEQ ID NO: 52) | (SEQ ID NO: 93) | (SEQ ID NO: 422) | |
| | LNHDA | SQIVND | ASSIEGQKCTLKL | 6 |
| | (SEQ ID NO: 47) | (SEQ ID NO: 109) | (SEQ ID NO: 423) | |
| | MDHEN | SYDVKM | ASSLSGSSYEQY | 4 |
| | (SEQ ID NO: 52) | (SEQ ID NO: 93) | (SEQ ID NO: 424) | |
| | MDHEN | SYDVKM | ASSLISGSSTSS | 4 |
| | (SEQ ID NO: 52) | (SEQ ID NO: 93) | (SEQ ID NO: 425) | |
| | MDHEN | SYDVKM | ASSLISGSSREQY | 4 |
| | (SEQ ID NO: 52) | (SEQ ID NO: 93) | (SEQ ID NO: 426) | |
| | MDHEN | SYDVKM | ASILISGSSYEQY | 3 |
| | (SEQ ID NO: 52) | (SEQ ID NO: 93) | (SEQ ID NO: 427) | |
| | LNHDA | SQIVND | ASSKEGQK*TLKL | 3 |
| | (SEQ ID NO: 47) | (SEQ ID NO: 109) | (SEQ ID NO: 428) | |
| | MDHEN | SYDVKM | ASTLISGSSYEQY | 2 |
| | (SEQ ID NO: 52) | (SEQ ID NO: 93) | (SEQ ID NO: 429) | |
| | MDHEN | SYDVKM | ASSPISGSPYEQY | 2 |
| | (SEQ ID NO: 52) | (SEQ ID NO: 93) | (SEQ ID NO: 430) | |
| | MDHEN | SYDVKM | ASSLVSGNSYEQY | 2 |
| | (SEQ ID NO: 52) | (SEQ ID NO: 93) | (SEQ ID NO: 431) | |
| | MDHEN | SYDVKM | ASSLISGSCYEQY | 2 |
| | (SEQ ID NO: 52) | (SEQ ID NO: 93) | (SEQ ID NO: 432) | |
| | MDHEN | SYDVKM | ASGPISGSSYEQY | 2 |
| | (SEQ ID NO: 52) | (SEQ ID NO: 93) | (SEQ ID NO: 433) | |
| | MDHEN | SYDVKM | ASGLISGGSYEQY | 2 |
| | (SEQ ID NO: 52) | (SEQ ID NO: 93) | (SEQ ID NO: 434) | |
| | MDHEN | SYDVKM | ASGLIGGSSYEQY | 2 |
| | (SEQ ID NO: 52) | (SEQ ID NO: 93) | (SEQ ID NO: 435) | |
| | LNHDA | SQVVND | ASSIEGQK*H*SF | 2 |
| | (SEQ ID NO: 47) | (SEQ ID NO: 119) | (SEQ ID NO: 436) | |
| | LNHDA | SQIVND | ASSREGQK*TLKL | 2 |
| | (SEQ ID NO: 47) | (SEQ ID NO: 109) | (SEQ ID NO: 437) | |
| | LNHDA | SQIVND | ASSIKGQK*TLKL | 2 |
| | (SEQ ID NO: 47) | (SEQ ID NO: 109) | (SEQ ID NO: 438) | |
| | LNHDA | SQIVND | ASSIEGQKGTLKL | 2 |
| | (SEQ ID NO: 47) | (SEQ ID NO: 109) | (SEQ ID NO: 439) | |
| TCRalpha | NYSPAY | IRENEKE | APPSGSARQLT | 687383 |
| Donor Y | (SEQ ID NO: 16) | (SEQ ID NO: 76) | (SEQ ID NO: 199) | |
| (CS11.6) | | | | |
| | NYSPAY | IRENEKE | APPPGSARQLT | 2142 |
| | (SEQ ID NO: 16) | (SEQ ID NO: 76) | (SEQ ID NO: 440) | |
| | SSVSVY | YLSGSTLV | AVMNAGKST | 1155 |
| | (SEQ ID NO: 7) | (SEQ ID NO: 67) | (SEQ ID NO: 441) | |
| | NYSPAY | IRENEKE | ALPSGSARQLT | 739 |
| | (SEQ ID NO: 16) | (SEQ ID NO: 76) | (SEQ ID NO: 442) | |
| | NYSPAY | IRENEKE | APPSSSARQLT | 652 |
| | (SEQ ID NO: 16) | (SEQ ID NO: 76) | (SEQ ID NO: 443) | |
| | NYSPAY | IRENEKE | TPPSGSARQLT | 644 |
| | (SEQ ID NO: 16) | (SEQ ID NO: 76) | (SEQ ID NO: 444) | |
| | NYSPAY | IRENEKE | VPPSGSARQLT | 450 |
| | (SEQ ID NO: 16) | (SEQ ID NO: 76) | (SEQ ID NO: 445) | |
| | NYSPAY | IRENEKE | APSSGSARQLT | 439 |
| | (SEQ ID NO: 16) | (SEQ ID NO: 76) | (SEQ ID NO: 446) | |
| | NYSPAY | IRENEKE | APPFGSARQLT | 411 |
| | (SEQ ID NO: 16) | (SEQ ID NO: 76) | (SEQ ID NO: 447) | |
| | NYSPAY | IRENEKE | APPTGSARQLT | 258 |
| | (SEQ ID NO: 16) | (SEQ ID NO: 76) | (SEQ ID NO: 448) | |
| | NSASDY | IRSNMDK | AENEDYGQNFV | 142 |
| | (SEQ ID NO: 25) | (SEQ ID NO: 98) | (SEQ ID NO: 449) | |
| | NYSPAY | IRENEKE | APPSGSARQL | 123 |
| | (SEQ ID NO: 16) | (SEQ ID NO: 76) | (SEQ ID NO: 450) | |
| | NYSPAY | IRENEKE | APPSGSARQLTFGSGTQL | 81 |
| | (SEQ ID NO: 16) | (SEQ ID NO: 76) | TVLPEHIKKRGEVTKGSL | |
| | | | L*GIKHCDTHGRRKQTH | |
| | | | (SEQ ID NO: 451) | |
| | NYSPAY | IRENEKE | AQPSGSARQLT | 77 |
| | (SEQ ID NO: 16) | (SEQ ID NO: 76) | (SEQ ID NO: 452) | |
| | NYSPAY | IRENEKE | APPSCSARQLT | 57 |
| | (SEQ ID NO: 16) | (SEQ ID NO: 76) | (SEQ ID NO: 453) | |
| | NYSPAY | IRENEKE | APPAGSARQLT | 56 |
| | (SEQ ID NO: 16) | (SEQ ID NO: 76) | (SEQ ID NO: 454) | |
| | NYSPAY | IRENEKE | APPYGSARQLT | 34 |
| | (SEQ ID NO: 16) | (SEQ ID NO: 76) | (SEQ ID NO: 455) | |
| | NYSPAY | IRENEKE | DPPSGSARQLT | 30 |
| | (SEQ ID NO: 16) | (SEQ ID NO: 76) | (SEQ ID NO: 456) | |
| | NYSPAY | IRENEKE | GPPSGSARQLT | 29 |
| | (SEQ ID NO: 16) | (SEQ ID NO: 76) | (SEQ ID NO: 457) | |
| | DRGSQS | IYSNGD | AVNIGGSQGNLI | 19 |
| | (SEQ ID NO: 13) | (SEQ ID NO: 73) | (SEQ ID NO: 458) | |
| | NYSPAY | IRENEKE | SPPSGSARQLT | 18 |
| | (SEQ ID NO: 16) | (SEQ ID NO: 76) | (SEQ ID NO: 459) | |
| | NYSPAY | IRENEKE | APPSRSARQLT | 16 |
| | (SEQ ID NO: 16) | (SEQ ID NO: 76) | (SEQ ID NO: 460) | |
| | NYSPAY | IRENEKE | ARPLVLQGN* | 14 |
| | (SEQ ID NO: 16) | (SEQ ID NO: 76) | (SEQ ID NO: 461) | |
| | NYSPAY | IRENEKE | PPPSGSARQLT | 13 |
| | (SEQ ID NO: 16) | (SEQ ID NO: 76) | (SEQ ID NO: 462) | |
| | NYSPAY | IRENEKE | APPSDSARQLT | 7 |
| | (SEQ ID NO: 16) | (SEQ ID NO: 76) | (SEQ ID NO: 463) | |
| | DRGSQS | IYSNGD | AVYSGYSTLT | 7 |
| | (SEQ ID NO: 13) | (SEQ ID NO: 73) | (SEQ ID NO: 464) | |
| | YGGTVN | YFSGDPLV | AVNARDSGTYKYI | 6 |
| | (SEQ ID NO: 26) | (SEQ ID NO: 99) | (SEQ ID NO: 465) | |
| | NTAFDY | IRPDVS | AAPGECWQQP*AD | 6 |
| | (SEQ ID NO: 27) | (SEQ ID NO: 120) | (SEQ ID NO: 466) | |
| | TSINN | IRSNERE | ARTGYSGGGADGLT | 4 |
| | (SEQ ID NO: 11) | (SEQ ID NO: 71) | (SEQ ID NO: 467) | |
| | TSESDYY | QEAYKQQN | AYDQGGSEKLV | 4 |
| | (SEQ ID NO: 14) | (SEQ ID NO: 74) | (SEQ ID NO: 468) | |
| | TSDQSYG | QGSYDEQN | AMSFRGGYQKVT | 4 |
| | (SEQ ID NO: 28) | (SEQ ID NO: 121) | (SEQ ID NO: 469) | |
| | NYSPAY | IRENEKE | APPSGPARQLT | 4 |
| | (SEQ ID NO: 16) | (SEQ ID NO: 76) | (SEQ ID NO: 470) | |
| | NYSPAY | IRENEKE | APPSDPARQLT | 4 |
| | (SEQ ID NO: 16) | (SEQ ID NO: 76) | (SEQ ID NO: 471) | |
| | NYSPAY | IRENEKE | APPCGSARQLT | 4 |
| | (SEQ ID NO: 16) | (SEQ ID NO: 76) | (SEQ ID NO: 472) | |
| | DSAIYN | IQSSQRE | AVRPIEHR*PVL | 4 |
| | (SEQ ID NO: 29) | (SEQ ID NO: 122) | (SEQ ID NO: 473) | |
| | YSGSPE | HISR | ALRSGGYQKVT | 3 |
| | (SEQ ID NO: 21) | (SEQ ID NO: 95) | (SEQ ID NO: 474) | |
| | YGATPY | YFSGDTLV | AVGAGGKLI | 3 |
| | (SEQ ID NO: 30) | (SEQ ID NO: 123) | (SEQ ID NO: 475) | |
| | SSVSVY | YLSGSTLV | AVTNAGKST | 3 |
| | (SEQ ID NO: 7) | (SEQ ID NO: 67) | (SEQ ID NO: 476) | |
| | SSVSVY | YLSGSTLV | AVMSAGKST | 3 |
| | (SEQ ID NO: 7) | (SEQ ID NO: 67) | (SEQ ID NO: 477) | |
| | SSVSVY | YLSGSTLV | AVMDAGKST | 3 |
| | (SEQ ID NO: 7) | (SEQ ID NO: 67) | (SEQ ID NO: 478) | |
| | NYSPAY | IRENEKE | PLVLQGN* | 3 |
| | (SEQ ID NO: 16) | (SEQ ID NO: 76) | (SEQ ID NO: 479) | |
| | NYSPAY | IRENEKE | APPSGSARQLTFGSGTQL | 3 |
| | (SEQ ID NO: 16) | (SEQ ID NO: 76) | TVLPEHIKKRGEVTKGSL | |
| | | | L*GIKHCETHGRRKQTH | |
| | | | (SEQ ID NO: 480) | |
| | NYSPAY | IRENEKE | APPPDSARQLT | 3 |
| | (SEQ ID NO: 16) | (SEQ ID NO: 76) | (SEQ ID NO: 481) | |
| | NSAFQY | TYSSGN | AMSLDNYGQNFV | 3 |
| | (SEQ ID NO: 23) | (SEQ ID NO: 97) | (SEQ ID NO: 482) | |
| | TSENNYY | QEAYKQQN | AFILQGAQKLV | 2 |
| | (SEQ ID NO: 31) | (SEQ ID NO: 74) | (SEQ ID NO: 483) | |
| | TRDTTYY | RNSFDEQN | ALELSGYALN | 2 |
| | (SEQ ID NO: 17) | (SEQ ID NO: 77) | (SEQ ID NO: 484) | |
| | TISGTDY | GLTSN | IWLRADLKSW | 2 |
| | (SEQ ID NO: 79) | (SEQ ID NO: 78) | (SEQ ID NO: 485) | |
| | SSVSVY | YLSGSTLV | AYGSSNTGKLI | 2 |
| | (SEQ ID NO: 7) | (SEQ ID NO: 67) | (SEQ ID NO: 486) | |
| | SSVSVY | YLSGSTLV | AVSARRQNFV | 2 |
| | (SEQ ID NO: 7) | (SEQ ID NO: 67) | (SEQ ID NO: 487) | |
| | SSVSVY | YLSGSTLV | AVRNAGKST | 2 |
| | (SEQ ID NO: 7) | (SEQ ID NO: 67) | (SEQ ID NO: 488) | |
| | SSVSVY | YLSGSTLV | AMMNAGKST | 2 |
| | (SEQ ID NO: 7) | (SEQ ID NO: 67) | (SEQ ID NO: 489) | |
| | NYSPAY | IRENEKE | VPSSGSARQLT | 2 |
| | (SEQ ID NO: 16) | (SEQ ID NO: 76) | (SEQ ID NO: 490) | |
| | NYSPAY | IRENEKE | TPPFGSARQLT | 2 |
| | (SEQ ID NO: 16) | (SEQ ID NO: 76) | (SEQ ID NO: 491) | |
| | NYSPAY | IRENEKE | TPLSGSARQLT | 2 |
| | (SEQ ID NO: 16) | (SEQ ID NO: 76) | (SEQ ID NO: 492) | |
| | NYSPAY | IRENEKE | SALWFCKATD | 2 |
| | (SEQ ID NO: 16) | (SEQ ID NO: 76) | (SEQ ID NO: 493) | |
| | NYSPAY | IRENEKE | ATPPGSARQLT | 2 |
| | (SEQ ID NO: 16) | (SEQ ID NO: 76) | (SEQ ID NO: 494) | |
| | NYSPAY | IRENEKE | ASPPGSARQLT | 2 |
| | (SEQ ID NO: 16) | (SEQ ID NO: 76) | (SEQ ID NO: 495) | |
| | NYSPAY | IRENEKE | APSSAGNNRKLI | 2 |
| | (SEQ ID NO: 16) | (SEQ ID NO: 76) | (SEQ ID NO: 496) | |
| | NYSPAY | IRENEKE | APPSVLQGN* | 2 |
| | (SEQ ID NO: 16) | (SEQ ID NO: 76) | (SEQ ID NO: 497) | |
| | NYSPAY | IRENEKE | APPSGSARQLTFGSGTQL | 2 |
| | (SEQ ID NO: 16) | (SEQ ID NO: 76) | AVLPEHIKKRGEVTKGSL | |
| | | | L*GIKHCDTHGRRKQTH | |
| | | | (SEQ ID NO: 498) | |
| | NYSPAY | IRENEKE | APPSGSAGNW | 2 |
| | (SEQ ID NO: 16) | (SEQ ID NO: 76) | (SEQ ID NO: 499) | |
| | NYSPAY | IRENEKE | APPSGLARQLT | 2 |
| | (SEQ ID NO: 16) | (SEQ ID NO: 76) | (SEQ ID NO: 500) | |
| | NYSPAY | IRENEKE | APPPGSARQL | 2 |
| | (SEQ ID NO: 16) | (SEQ ID NO: 76) | (SEQ ID NO: 501) | |
| | NYSPAY | IRENEKE | APPLGSARQLT | 2 |
| | (SEQ ID NO: 16) | (SEQ ID NO: 76) | (SEQ ID NO: 502) | |
| | NYSPAY | IRENEKE | ALPPGSARQLT | 2 |
| | (SEQ ID NO: 16) | (SEQ ID NO: 76) | (SEQ ID NO: 503) | |
| | NYSPAY | IRENEKE | ALDLTGNQFY | 2 |
| | (SEQ ID NO: 16) | (SEQ ID NO: 76) | (SEQ ID NO: 504) | |
| | NSAFQY | TYSSGN | AASLSNFGNEKLT | 2 |
| | (SEQ ID NO: 23) | (SEQ ID NO: 97) | (SEQ ID NO: 505) | |
| | DSVNN | IPSGT | AVDNYGQNFV | 2 |
| | (SEQ ID NO: 6) | (SEQ ID NO: 66) | (SEQ ID NO: 130) | |
| | DSASNY | IRSNVGE | AASFSDQTGANNLF | 2 |
| | (SEQ ID NO: 24) | (SEQ ID NO: 80) | (SEQ ID NO: 506) | |
| TCRbeta | KGHSH | LQKENI | ASSPPEGFGNEQF | 587843 |
| Donor Y | (SEQ ID NO: 38) | (SEQ ID NO: 81) | (SEQ ID NO: 245) | |
| (CS11.6) | | | | |
| | KGHSH | LQKENI | ASSPPGGFGNEQF | 1829 |
| | (SEQ ID NO: 38) | (SEQ ID NO: 81) | (SEQ ID NO: 325) | |
| | KGHSH | LQKENI | ASSPPEGFGDEQF | 1580 |
| | (SEQ ID NO: 38) | (SEQ ID NO: 81) | (SEQ ID NO: 507) | |
| | KGHSH | LQKENI | ASSPPEGLGNEQF | 1481 |
| | (SEQ ID NO: 38) | (SEQ ID NO: 81) | (SEQ ID NO: 349) | |
| | KGHSH | LQKENI | ASSPPEGFGSEQF | 1274 |
| | (SEQ ID NO: 38) | (SEQ ID NO: 81) | (SEQ ID NO: 508) | |
| | KGHSH | LQKENI | ASSPPEGSGNEQF | 1083 |
| | (SEQ ID NO: 38) | (SEQ ID NO: 81) | (SEQ ID NO: 365) | |
| | KGHSH | LQKENI | ASSPPEGFDNEQF | 640 |
| | (SEQ ID NO: 38) | (SEQ ID NO: 81) | (SEQ ID NO: 509) | |
| | KGHSH | LQKENI | ASSLPEGFGNEQF | 491 |
| | (SEQ ID NO: 38) | (SEQ ID NO: 81) | (SEQ ID NO: 510) | |
| | KGHSH | LQKENI | ASSPLEGFGNEQF | 451 |
| | (SEQ ID NO: 38) | (SEQ ID NO: 81) | (SEQ ID NO: 511) | |
| | KGHSH | LQKENI | ASSPSEGFGNEQF | 381 |
| | (SEQ ID NO: 38) | (SEQ ID NO: 81) | (SEQ ID NO: 512) | |
| | KGHSH | LQKENI | ASSPPEGLAMSS | 357 |
| | (SEQ ID NO: 38) | (SEQ ID NO: 81) | (SEQ ID NO: 513) | |
| | KGHSH | LQKENI | ASSHLRVLAMSS | 259 |
| | (SEQ ID NO: 38) | (SEQ ID NO: 81) | (SEQ ID NO: 514) | |
| | MNHEY | SMNVEV | ASSPPGLGYEQY | 186 |
| | (SEQ ID NO: 42) | (SEQ ID NO: 115) | (SEQ ID NO: 515) | |
| | SGHRS | YFSETQ | ASSPRGGSYEQY | 173 |
| | (SEQ ID NO: 59) | (SEQ ID NO: 124) | (SEQ ID NO: 516) | |
| | KGHSH | LQKENI | ASSPPEGFGKEQF | 146 |
| | (SEQ ID NO: 38) | (SEQ ID NO: 81) | (SEQ ID NO: 517) | |
| | ENHRY | SYGVKD | AIRSTASTDTQY | 138 |
| | (SEQ ID NO: 48) | (SEQ ID NO: 110) | (SEQ ID NO: 518) | |
| | KGHSH | LQKENI | ASSPHEGFGNEQF | 130 |
| | (SEQ ID NO: 38) | (SEQ ID NO: 81) | (SEQ ID NO: 519) | |
| | KGHSH | LQKENI | AAHHLRVLAMSS | 111 |
| | (SEQ ID NO: 38) | (SEQ ID NO: 81) | (SEQ ID NO: 520) | |
| | KGHSH | LQKENI | ASSPPEGFGYEQF | 83 |
| | (SEQ ID NO: 38) | (SEQ ID NO: 81) | (SEQ ID NO: 521) | |
| | KGHSH | LQKENI | ASSQPEGFGNEQF | 81 |
| | (SEQ ID NO: 38) | (SEQ ID NO: 81) | (SEQ ID NO: 522) | |
| | MRHNA | SNTAGT | ASRGTSVQQ*AV | 57 |
| | (SEQ ID NO: 60) | (SEQ ID NO: 125) | (SEQ ID NO: 523) | |
| | SGHDY | FNNNVP | ASTPSGPSTDTQY | 53 |
| | (SEQ ID NO: 37) | (SEQ ID NO: 103) | (SEQ ID NO: 524) | |
| | KGHSH | LQKENI | ASSPPEGFGIEQF | 53 |
| | (SEQ ID NO: 38) | (SEQ ID NO: 81) | (SEQ ID NO: 525) | |
| | MNHEY | SVGAGI | ASSYSGAGGPWDTQY | 49 |
| | (SEQ ID NO: 42) | (SEQ ID NO: 88) | (SEQ ID NO: 526) | |
| | KGHSH | LQKENI | ASSPPEGFGTEQF | 45 |
| | (SEQ ID NO: 38) | (SEQ ID NO: 81) | (SEQ ID NO: 527) | |
| | | | ASSVEGTGTSIQY | 45 |
| | | | (SEQ ID NO: 528) | |
| | KGHSH | LQKENI | ASSPPEGFGHEQF | 37 |
| | (SEQ ID NO: 38) | (SEQ ID NO: 81) | (SEQ ID NO: 529) | |
| | KGHSH | LQKENI | ASSPTEGFGNEQF | 26 |
| | (SEQ ID NO: 38) | (SEQ ID NO: 81) | (SEQ ID NO: 530) | |
| | SGHVS | FNYEAQ | ASSLWGTEAF | 18 |
| | (SEQ ID NO: 43) | (SEQ ID NO: 106) | (SEQ ID NO: 531) | |
| | KGHSH | LQKENI | ASSRPEGFGNEQF | 18 |
| | (SEQ ID NO: 38) | (SEQ ID NO: 81) | (SEQ ID NO: 532) | |
| | KGHSH | LQKENI | ASSPREGFGNEQF | 18 |
| | (SEQ ID NO: 38) | (SEQ ID NO: 81) | (SEQ ID NO: 533) | |
| | MNHEY | SMNVEV | ASSLDRLYTEAF | 12 |
| | (SEQ ID NO: 42) | (SEQ ID NO: 115) | (SEQ ID NO: 534) | |
| | PRHDT | FYEKMQ | ASSFGTGGNTQY | 9 |
| | (SEQ ID NO: 61) | (SEQ ID NO: 126) | (SEQ ID NO: 535) | |
| | KGHSH | LQKENI | ASSPAEGFGNEQF | 9 |
| | (SEQ ID NO: 38) | (SEQ ID NO: 81) | (SEQ ID NO: 536) | |
| | MNHEY | SVGAGI | ASSLYFGQPQH | 7 |
| | (SEQ ID NO: 42) | (SEQ ID NO: 88) | (SEQ ID NO: 537) | |
| | MNHEY | SVGAGI | ASRTEAREQY | 6 |
| | (SEQ ID NO: 42) | (SEQ ID NO: 88) | (SEQ ID NO: 538) | |
| | LGHDT | YNNKEL | ASSQPGQYGYT | 6 |
| | (SEQ ID NO: 51) | (SEQ ID NO: 114) | (SEQ ID NO: 539) | |
| | KGHSH | LQKENI | ASSPPEGFGDGQF | 6 |
| | (SEQ ID NO: 38) | (SEQ ID NO: 81) | (SEQ ID NO: 540) | |
| | SGHDY | FNNNVP | ASRLGHQPQH | 5 |
| | (SEQ ID NO: 37) | (SEQ ID NO: 103) | (SEQ ID NO: 541) | |
| | KGHSH | LQKENI | ASSPPKGFGNEQF | 5 |
| | (SEQ ID NO: 38) | (SEQ ID NO: 81) | (SEQ ID NO: 542) | |
| | KGHSH | LQKENI | ASSPPEGFGNRQF | 5 |
| | (SEQ ID NO: 38) | (SEQ ID NO: 81) | (SEQ ID NO: 543) | |
| | KGHSH | LQKENI | ASPPPEGFGNEQF | 5 |
| | (SEQ ID NO: 38) | (SEQ ID NO: 81) | (SEQ ID NO: 544) | |
| | SQVTM | ANQGSEA | SVGVTGGTITPHEQY | 4 |
| | (SEQ ID NO: 36) | (SEQ ID NO: 90) | (SEQ ID NO: 545) | |
| | SGHRS | YFSETQ | ASSDRDRDG*RARGGEQF | 4 |
| | (SEQ ID NO: 59) | (SEQ ID NO: 124) | (SEQ ID NO: 546) | |
| | KGHSH | LQKENI | ASSPPKGLGNEQF | 4 |
| | (SEQ ID NO: 38) | (SEQ ID NO: 81) | (SEQ ID NO: 547) | |
| | KGHSH | LQKENI | ASSPPESLGNEQF | 4 |
| | (SEQ ID NO: 38) | (SEQ ID NO: 81) | (SEQ ID NO: 548) | |
| | KGHSH | LQKENI | ASSPPEGFGNKQF | 4 |
| | (SEQ ID NO: 38) | (SEQ ID NO: 81) | (SEQ ID NO: 549) | |
| | GTSNPN | SVGIG | AWRPGYMNTEAF | 4 |
| | (SEQ ID NO: 40) | (SEQ ID NO: 104) | (SEQ ID NO: 550) | |
| | ENHRY | SYGVKD | AISEWASGRPSYEQY | 4 |
| | (SEQ ID NO: 48) | (SEQ ID NO: 110) | (SEQ ID NO: 551) | |
| | | | ASSALAGDTYEQY | 4 |
| | | | (SEQ ID NO: 552) | |
| | SGHRS | YFSETQ | ASSPRRGSYEQY | 3 |
| | (SEQ ID NO: 59) | (SEQ ID NO: 124) | (SEQ ID NO: 553) | |
| | SGHRS | YFSETQ | ASSPRGAPTSS | 3 |
| | (SEQ ID NO: 59) | (SEQ ID NO: 124) | (SEQ ID NO: 554) | |
| | MDHEY | SVGAGI | ASSYSPGNHQPQH | 3 |
| | (SEQ ID NO: 62) | (SEQ ID NO: 88) | (SEQ ID NO: 555) | |
| | KGHSH | LQKENI | ASSPSEGFDNEQF | 3 |
| | (SEQ ID NO: 38) | (SEQ ID NO: 81) | (SEQ ID NO: 556) | |
| | KGHSH | LQKENI | ASSPPEGLSNEQF | 3 |
| | (SEQ ID NO: 38) | (SEQ ID NO: 81) | (SEQ ID NO: 557) | |
| | KGHSH | LQKENI | ASSPPEGFSSEQF | 3 |
| | (SEQ ID NO: 38) | (SEQ ID NO: 81) | (SEQ ID NO: 558) | |
| | KGHSH | LQKENI | ASSPPEGFGNE*F | 3 |
| | (SEQ ID NO: 38) | (SEQ ID NO: 81) | (SEQ ID NO: 559) | |
| | KGHSH | LQKENI | ASSPPEDFGNEQF | 3 |
| | (SEQ ID NO: 38) | (SEQ ID NO: 81) | (SEQ ID NO: 560) | |
| | ENHRY | SYGVKD | AIRSTASADTQY | 3 |
| | (SEQ ID NO: 48) | (SEQ ID NO: 110) | (SEQ ID NO: 561) | |
| | SNHLY | FYNNEI | ATTRTSGSNEQF | 2 |
| | (SEQ ID NO: 44) | (SEQ ID NO: 94) | (SEQ ID NO: 562) | |
| | SGHRS | YFSETQ | ASSPRGGLLRAV | 2 |
| | (SEQ ID NO: 59) | (SEQ ID NO: 124) | (SEQ ID NO: 563) | |
| | MNHEY | SVGEGT | ASSYGLAHSYEQY | 2 |
| | (SEQ ID NO: 42) | (SEQ ID NO: 91) | (SEQ ID NO: 564) | |
| | MNHEY | SMNVEV | ATLQGPNEQF | 2 |
| | (SEQ ID NO: 42) | (SEQ ID NO: 115) | (SEQ ID NO: 565) | |
| | MDHEN | SYDVKM | ASSSSVLRARTEAF | 2 |
| | (SEQ ID NO: 52) | (SEQ ID NO: 93) | (SEQ ID NO: 566) | |
| | LNHDA | SQIVND | ASSIFLGDNTGELF | 2 |
| | (SEQ ID NO: 47) | (SEQ ID NO: 109) | (SEQ ID NO: 567) | |
| | KGHSR | LQKENI | ASLPPEGFGNEQF | 2 |
| | (SEQ ID NO: 63) | (SEQ ID NO: 81) | (SEQ ID NO: 568) | |
| | KGHSH | LQKENI | ASSPSEDFGNEQF | 2 |
| | (SEQ ID NO: 38) | (SEQ ID NO: 81) | (SEQ ID NO: 569) | |
| | KGHSH | LQKENI | ASSPPKGSGNEQF | 2 |
| | (SEQ ID NO: 38) | (SEQ ID NO: 81) | (SEQ ID NO: 570) | |
| | KGHSH | LQKENI | ASSPPGGFDNEQF | 2 |
| | (SEQ ID NO: 38) | (SEQ ID NO: 81) | (SEQ ID NO: 571) | |
| | KGHSH | LQKENI | ASSPPEGFSNEQF | 2 |
| | (SEQ ID NO: 38) | (SEQ ID NO: 81) | (SEQ ID NO: 366) | |
| | KGHSH | LQKENI | ASSPPEGFNNEQF | 2 |
| | (SEQ ID NO: 38) | (SEQ ID NO: 81) | (SEQ ID NO: 572) | |
| | KGHSH | LQKENI | ASSPPEGFGYGQF | 2 |
| | (SEQ ID NO: 38) | (SEQ ID NO: 81) | (SEQ ID NO: 573) | |
| | KGHSH | LQKENI | ASSPPEGFGNGQF | 2 |
| | (SEQ ID NO: 38) | (SEQ ID NO: 81) | (SEQ ID NO: 574) | |
| | KGHSH | LQKENI | ASSPPEDSGNEQF | 2 |
| | (SEQ ID NO: 38) | (SEQ ID NO: 81) | (SEQ ID NO: 575) | |
| | KGHSH | LQKENI | ASSPPEDFDNEQF | 2 |
| | (SEQ ID NO: 38) | (SEQ ID NO: 81) | (SEQ ID NO: 576) | |
| | KGHSH | LQKENI | ASSPLKGFGNEQF | 2 |
| | (SEQ ID NO: 38) | (SEQ ID NO: 81) | (SEQ ID NO: 577) | |
| | KGHSH | LQKENI | ASSPLGGFGNEQF | 2 |
| | (SEQ ID NO: 38) | (SEQ ID NO: 81) | (SEQ ID NO: 578) | |
| | KGHSH | LQKENI | ASSPLEGFSNEQF | 2 |
| | (SEQ ID NO: 38) | (SEQ ID NO: 81) | (SEQ ID NO: 579) | |
| | KGHSH | LQKENI | ASSPLEDFGNEQF | 2 |
| | (SEQ ID NO: 38) | (SEQ ID NO: 81) | (SEQ ID NO: 580) | |
| | KGHSH | LQKENI | ASSLPEGFDNEQF | 2 |
| | (SEQ ID NO: 38) | (SEQ ID NO: 81) | (SEQ ID NO: 581) | |
| | KGHSH | LQKENI | ASPLPEGFGNEQF | 2 |
| | (SEQ ID NO: 38) | (SEQ ID NO: 81) | (SEQ ID NO: 582) | |
| | KGHSH | LQKENI | ASLPPEGFGNEQF | 2 |
| | (SEQ ID NO: 38) | (SEQ ID NO: 81) | (SEQ ID NO: 568) | |
| | DFQATT | SNEGSKA | SVWTDSDTQY | 2 |
| | (SEQ ID NO: 39) | (SEQ ID NO: 89) | (SEQ ID NO: 583) | |
| | | | ASSGPSGQPQH | 2 |
| | | | (SEQ ID NO: 584) | |
| | | | ASLSSPPRDPWRLIHPS | 2 |
| | | | (SEQ ID NO: 585) | |
| TCRalpha | NYSPAY | IRENEKE | APYTGRRALT | 60408 |
| Donor Y | (SEQ ID NO: 16) | (SEQ ID NO: 76) | (SEQ ID NO: 223) | |
| (GV10.3) | | | | |
| | | | AASGANTNKVV | 464 |
| | | | (SEQ ID NO: 586) | |
| | NYSPAY | IRENEKE | APYAGRRALT | 169 |
| | (SEQ ID NO: 16) | (SEQ ID NO: 76) | (SEQ ID NO: 587) | |
| | NYSPAY | IRENEKE | APCTGRRALT | 107 |
| | (SEQ ID NO: 16) | (SEQ ID NO: 76) | (SEQ ID NO: 588) | |
| | NYSPAY | IRENEKE | TPYTGRRALT | 61 |
| | (SEQ ID NO: 16) | (SEQ ID NO: 76) | (SEQ ID NO: 589) | |
| | NYSPAY | IRENEKE | VPYTGRRALT | 59 |
| | (SEQ ID NO: 16) | (SEQ ID NO: 76) | (SEQ ID NO: 590) | |
| | NYSPAY | IRENEKE | APYMGRRALT | 57 |
| | (SEQ ID NO: 16) | (SEQ ID NO: 76) | (SEQ ID NO: 591) | |
| | NYSPAY | IRENEKE | APYTGRRAL | 39 |
| | (SEQ ID NO: 16) | (SEQ ID NO: 76) | (SEQ ID NO: 592) | |
| | SIFNT | LYKAGEL | AGQDQDSGYALN | 36 |
| | (SEQ ID NO: 20) | (SEQ ID NO: 84) | (SEQ ID NO: 593) | |
| | NYSPAY | IRENEKE | APTRAGEHL | 16 |
| | (SEQ ID NO: 16) | (SEQ ID NO: 76) | (SEQ ID NO: 594) | |
| | | IFSNGE | AASEGNYNVLY | 15 |
| | | (SEQ ID NO: 85) | (SEQ ID NO: 386) | |
| | DSVNN | IPSGT | AANSNDYKLS | 9 |
| | (SEQ ID NO: 6) | (SEQ ID NO: 66) | (SEQ ID NO: 595) | |
| | NYSPAY | IRENEKE | APYPGRRALT | 7 |
| | (SEQ ID NO: 16) | (SEQ ID NO: 76) | (SEQ ID NO: 596) | |
| | NYSPAY | IRENEKE | APYKGRRALT | 7 |
| | (SEQ ID NO: 16) | (SEQ ID NO: 76) | (SEQ ID NO: 597) | |
| | NYSPAY | IRENEKE | APYSGRRALT | 6 |
| | (SEQ ID NO: 16) | (SEQ ID NO: 76) | (SEQ ID NO: 598) | |
| | NYSPAY | IRENEKE | ALLDSGGGADGLT | 6 |
| | (SEQ ID NO: 16) | (SEQ ID NO: 76) | (SEQ ID NO: 599) | |
| | NYSPAY | IRENEKE | GPYTGRRALT | 5 |
| | (SEQ ID NO: 16) | (SEQ ID NO: 76) | (SEQ ID NO: 600) | |
| | | | AASGANTNKAV | 5 |
| | | | (SEQ ID NO: 601) | |
| | NYSPAY | IRENEKE | APSTGRRALT | 4 |
| | (SEQ ID NO: 16) | (SEQ ID NO: 76) | (SEQ ID NO: 602) | |
| | NYSPAY | IRENEKE | AP*TGRRALT | 4 |
| | (SEQ ID NO: 16) | (SEQ ID NO: 76) | (SEQ ID NO: 603) | |
| | SSVSVY | YLSGSTLV | AVSAITQGGSEKLV | 3 |
| | (SEQ ID NO: 7) | (SEQ ID NO: 67) | (SEQ ID NO: 604) | |
| | NYSPAY | IRENEKE | SPYTGRRALT | 3 |
| | (SEQ ID NO: 16) | (SEQ ID NO: 76) | (SEQ ID NO: 605) | |
| | NYSPAY | IRENEKE | PPYTGRRALT | 3 |
| | (SEQ ID NO: 16) | (SEQ ID NO: 76) | (SEQ ID NO: 606) | |
| | NYSPAY | IRENEKE | EPYTGRRALT | 3 |
| | (SEQ ID NO: 16) | (SEQ ID NO: 76) | (SEQ ID NO: 607) | |
| | NYSPAY | IRENEKE | APYTAGEHL | 3 |
| | (SEQ ID NO: 16) | (SEQ ID NO: 76) | (SEQ ID NO: 608) | |
| | DSSSTY | IFSNMDM | AGPGG*QL*ID | 3 |
| | (SEQ ID NO: 64) | (SEQ ID NO: 86) | (SEQ ID NO: 609) | |
| | NYSPAY | IRENEKE | APSRAGEHL | 2 |
| | (SEQ ID NO: 16) | (SEQ ID NO: 76) | (SEQ ID NO: 610) | |
| | DSSSTY | IFSNMDM | AERYNTDKLI | 2 |
| | (SEQ ID NO: 64) | (SEQ ID NO: 86) | (SEQ ID NO: 611) | |
| | | | AASGANTNKFV | 2 |
| | | | (SEQ ID NO: 612) | |
| TCRbeta | SGHAT | FQDESV | ASSLGQGNEAF | 72824 |
| Donor Y | (SEQ ID NO: 46) | (SEQ ID NO: 87) | (SEQ ID NO: 613) | |
| (GV10.3) | | | | |
| | MNHEY | SVGAGI | ASSLYFGQPQH | 36151 |
| | (SEQ ID NO: 42) | (SEQ ID NO: 88) | (SEQ ID NO: 537) | |
| | DFQATT | SNEGSKA | SAREGSGNEQF | 395 |
| | (SEQ ID NO: 39) | (SEQ ID NO: 89) | (SEQ ID NO: 614) | |
| | SGHAT | FQDESV | ASSLGQGDEAF | 276 |
| | (SEQ ID NO: 46) | (SEQ ID NO: 87) | (SEQ ID NO: 615) | |
| | SGHAT | FQDESV | ASSLGRGNEAF | 263 |
| | (SEQ ID NO: 46) | (SEQ ID NO: 87) | (SEQ ID NO: 616) | |
| | SGHAT | FQDESV | ASSLGQGSEAF | 247 |
| | (SEQ ID NO: 46) | (SEQ ID NO: 87) | (SEQ ID NO: 617) | |
| | SGHAT | FQDESV | ASSLGQGNGAF | 243 |
| | (SEQ ID NO: 46) | (SEQ ID NO: 87) | (SEQ ID NO: 618) | |
| | SGHAT | FQDESV | ASSSGQGNEAF | 202 |
| | (SEQ ID NO: 46) | (SEQ ID NO: 87) | (SEQ ID NO: 619) | |
| | MNHEY | SVGAGI | ASSLYLGQPQH | 132 |
| | (SEQ ID NO: 42) | (SEQ ID NO: 88) | (SEQ ID NO: 620) | |
| | SQVTM | ANQGSEA | SASSGSTDTQY | 125 |
| | (SEQ ID NO: 36) | (SEQ ID NO: 90) | (SEQ ID NO: 621) | |
| | MNHEY | SVGAGI | ASSLYFGRPQH | 114 |
| | (SEQ ID NO: 42) | (SEQ ID NO: 88) | (SEQ ID NO: 622) | |
| | MNHEY | SVGAGI | ASSLYSGQPQH | 110 |
| | (SEQ ID NO: 42) | (SEQ ID NO: 88) | (SEQ ID NO: 623) | |
| | MNHEY | SVGAGI | ASGLYFGQPQH | 107 |
| | (SEQ ID NO: 42) | (SEQ ID NO: 88) | (SEQ ID NO: 624) | |
| | MNHEY | SVGAGI | ASSLYFGQPS | 76 |
| | (SEQ ID NO: 42) | (SEQ ID NO: 88) | (SEQ ID NO: 625) | |
| | SGHAT | FQDESV | ASSLGQGNKAF | 73 |
| | (SEQ ID NO: 46) | (SEQ ID NO: 87) | (SEQ ID NO: 626) | |
| | MNHEY | SVGAGI | ASSLCFGQPQH | 68 |
| | (SEQ ID NO: 42) | (SEQ ID NO: 88) | (SEQ ID NO: 627) | |
| | SQVTM | ANQGSEA | SVAGTYSYNEQF | 66 |
| | (SEQ ID NO: 36) | (SEQ ID NO: 90) | (SEQ ID NO: 628) | |
| | DFQATT | SNEGSKA | SARDQIREQF | 63 |
| | (SEQ ID NO: 39) | (SEQ ID NO: 89) | (SEQ ID NO: 629) | |
| | MNHEY | SVGEGT | ASSDDPRESGANVLT | 51 |
| | (SEQ ID NO: 42) | (SEQ ID NO: 91) | (SEQ ID NO: 630) | |
| | SGHAT | FQDESV | ASNLGQGNEAF | 45 |
| | (SEQ ID NO: 46) | (SEQ ID NO: 87) | (SEQ ID NO: 631) | |
| | SGHAT | FQDESV | ASSLDRGMKL | 44 |
| | (SEQ ID NO: 46) | (SEQ ID NO: 87) | (SEQ ID NO: 632) | |
| | SQVTM | ANQGSEA | SVEGTGGLNEQF | 41 |
| | (SEQ ID NO: 36) | (SEQ ID NO: 90) | (SEQ ID NO: 633) | |
| | SGHAT | FQDESV | ASSLGQGMKL | 39 |
| | (SEQ ID NO: 46) | (SEQ ID NO: 87) | (SEQ ID NO: 634) | |
| | MNHNS | SASEGT | ASSLGWRGNSYEQY | 39 |
| | (SEQ ID NO: 53) | (SEQ ID NO: 92) | (SEQ ID NO: 635) | |
| | SGHAT | FQDESV | ASS*GQGNEAF | 34 |
| | (SEQ ID NO: 46) | (SEQ ID NO: 87) | (SEQ ID NO: 636) | |
| | MNHEY | SVGAGI | ASNLYFGQPQH | 33 |
| | (SEQ ID NO: 42) | (SEQ ID NO: 88) | (SEQ ID NO: 637) | |
| | MNHEY | SVGAGI | ASSLYFGQPQ | 31 |
| | (SEQ ID NO: 42) | (SEQ ID NO: 88) | (SEQ ID NO: 638) | |
| | MNHEY | SVGAGI | ASSLYFG*PQH | 30 |
| | (SEQ ID NO: 42) | (SEQ ID NO: 88) | (SEQ ID NO: 639) | |
| | SGHAT | FQNNGV | ASSLVSGGNEQ | 29 |
| | (SEQ ID NO: 46) | (SEQ ID NO: 108) | (SEQ ID NO: 640) | |
| | MNHEY | SVGAGI | ASSLYFGQSQH | 29 |
| | (SEQ ID NO: 42) | (SEQ ID NO: 88) | (SEQ ID NO: 641) | |
| | GTSNPN | SVGIG | AWEEGEAF | 29 |
| | (SEQ ID NO: 40) | (SEQ ID NO: 104) | (SEQ ID NO: 642) | |
| | SGHAT | FQDESV | ASSLGQGNEA | 27 |
| | (SEQ ID NO: 46) | (SEQ ID NO: 87) | (SEQ ID NO: 643) | |
| | SGHAT | FQDESV | ASSLGQGNVAF | 26 |
| | (SEQ ID NO: 46) | (SEQ ID NO: 87) | (SEQ ID NO: 644) | |
| | SGHAT | FQDESV | ASSLGQGKEAF | 26 |
| | (SEQ ID NO: 46) | (SEQ ID NO: 87) | (SEQ ID NO: 645) | |
| | SQVTM | ANQGSEA | SVDLGWEQY | 25 |
| | (SEQ ID NO: 36) | (SEQ ID NO: 90) | (SEQ ID NO: 646) | |
| | KGHDR | SFDVKD | ATSDLTGGNEQF | 24 |
| | (SEQ ID NO: 65) | (SEQ ID NO: 127) | (SEQ ID NO: 647) | |
| | SQVTM | ANQGSEA | SVELAGEADTQY | 19 |
| | (SEQ ID NO: 36) | (SEQ ID NO: 90) | (SEQ ID NO: 648) | |
| | MNHEY | SVGAGI | AAVYTLGSPS | 18 |
| | (SEQ ID NO: 42) | (SEQ ID NO: 88) | (SEQ ID NO: 649) | |
| | SGHAT | FQDESV | ASSMGQGNEAF | 17 |
| | (SEQ ID NO: 46) | (SEQ ID NO: 87) | (SEQ ID NO: 650) | |
| | MNHEY | SVGAGI | ASSYTLGSPS | 17 |
| | (SEQ ID NO: 42) | (SEQ ID NO: 88) | (SEQ ID NO: 651) | |
| | SGHAT | FQDESV | ASSLGQGNDAF | 16 |
| | (SEQ ID NO: 46) | (SEQ ID NO: 87) | (SEQ ID NO: 652) | |
| | MNHEY | SVGAGI | ASSLYFGSPS | 16 |
| | (SEQ ID NO: 42) | (SEQ ID NO: 88) | (SEQ ID NO: 653) | |
| | DFQATT | SNEGSKA | SASSGTSGRLYNEQF | 16 |
| | (SEQ ID NO: 39) | (SEQ ID NO: 89) | (SEQ ID NO: 654) | |
| | SGHAT | FQDESV | AAAWDRGMKL | 15 |
| | (SEQ ID NO: 46) | (SEQ ID NO: 87) | (SEQ ID NO: 655) | |
| | SGHAT | FQDESV | ASSWGQGNEAF | 13 |
| | (SEQ ID NO: 46) | (SEQ ID NO: 87) | (SEQ ID NO: 656) | |
| | SGHAT | FQDESV | ASSVGQGNEAF | 13 |
| | (SEQ ID NO: 46) | (SEQ ID NO: 87) | (SEQ ID NO: 657) | |
| | SEHNR | FQNEAQ | ASSLTLQETQY | 13 |
| | (SEQ ID NO: 56) | (SEQ ID NO: 117) | (SEQ ID NO: 658) | |
| | SQVTM | ANQGSEA | SVGTSGYEQY | 12 |
| | (SEQ ID NO: 36) | (SEQ ID NO: 90) | (SEQ ID NO: 659) | |
| | MNHEY | SVGAGI | ASSYFAGPYEQY | 11 |
| | (SEQ ID NO: 42) | (SEQ ID NO: 88) | (SEQ ID NO: 660) | |
| | GTSNPN | SVGIG | AWSEGVGNQPQH | 11 |
| | (SEQ ID NO: 40) | (SEQ ID NO: 104) | (SEQ ID NO: 661) | |
| | MNHEY | SVGAGI | ASS*YFGQPQH | 7 |
| | (SEQ ID NO: 42) | (SEQ ID NO: 88) | (SEQ ID NO: 662) | |
| | SGHAT | FQDESV | ASSLGQGNAAF | 6 |
| | (SEQ ID NO: 46) | (SEQ ID NO: 87) | (SEQ ID NO: 663) | |
| | SGHAT | FQDESV | ASILGQGNEAF | 6 |
| | (SEQ ID NO: 46) | (SEQ ID NO: 87) | (SEQ ID NO: 664) | |
| | SEHNR | FQNEAQ | ASSLVGAQGLAGTNNYEQ | 6 |
| | (SEQ ID NO: 56) | (SEQ ID NO: 117) | Y (SEQ ID NO: 665) | |
| | MNHEY | SVGAGI | ASSLYFGPPQH | 6 |
| | (SEQ ID NO: 42) | (SEQ ID NO: 88) | (SEQ ID NO: 666) | |
| | MNHEY | SVGAGI | ASSLYFGLPQH | 6 |
| | (SEQ ID NO: 42) | (SEQ ID NO: 88) | (SEQ ID NO: 667) | |
| | MNHEY | SVGAGI | ASSLYFGHPQH | 6 |
| | (SEQ ID NO: 42) | (SEQ ID NO: 88) | (SEQ ID NO: 668) | |
| | MNHEY | SVGAGI | ASSIYFGQPQH | 6 |
| | (SEQ ID NO: 42) | (SEQ ID NO: 88) | (SEQ ID NO: 669) | |
| | MNHEY | SVGAGI | ASILYFGQPQH | 6 |
| | (SEQ ID NO: 42) | (SEQ ID NO: 88) | (SEQ ID NO: 670) | |
| | MDHEN | SYDVKM | ASSPGSAYNEQF | 6 |
| | (SEQ ID NO: 52) | (SEQ ID NO: 93) | (SEQ ID NO: 671) | |
| | ENHRY | SYGVKD | AISESLAGGYNEQF | 6 |
| | (SEQ ID NO: 48) | (SEQ ID NO: 110) | (SEQ ID NO: 672) | |
| | SGHAT | FQDESV | ASRLGQGNEAF | 5 |
| | (SEQ ID NO: 46) | (SEQ ID NO: 87) | (SEQ ID NO: 673) | |
| | MNHEY | SMNVEV | ASSPTLGVDTQY | 5 |
| | (SEQ ID NO: 42) | (SEQ ID NO: 115) | (SEQ ID NO: 674) | |
| | | | ASSVDGGEQPQH | 5 |
| | | | (SEQ ID NO: 675) | |
| | SGHDN | FVKESK | ASSQYVEQY | 4 |
| | (SEQ ID NO: 41) | (SEQ ID NO: 105) | (SEQ ID NO: 676) | |
| | MNHEY | SVGAGI | ASSQGSDEQY | 4 |
| | (SEQ ID NO: 42) | (SEQ ID NO: 88) | (SEQ ID NO: 677) | |
| | MDHEN | SYDVKM | ASSLTGHREAYNEQF | 4 |
| | (SEQ ID NO: 52) | (SEQ ID NO: 93) | (SEQ ID NO: 678) | |
| | WSHSY | SAAADI | ASSSTGGTSYGYT | 3 |
| | (SEQ ID NO: 49) | (SEQ ID NO: 111) | (SEQ ID NO: 679) | |
| | SGHAT | FQDESV | ASSLGQGTEAF | 3 |
| | (SEQ ID NO: 46) | (SEQ ID NO: 87) | (SEQ ID NO: 680) | |
| | SGHAT | FQDESV | ASSLGQGN*AF | 3 |
| | (SEQ ID NO: 46) | (SEQ ID NO: 87) | (SEQ ID NO: 681) | |
| | SGHAT | FQDESV | ASSLGQGN | 3 |
| | (SEQ ID NO: 46) | (SEQ ID NO: 87) | (SEQ ID NO: 682) | |
| | SGHAT | FQDESV | ASSLGQGDGAF | 3 |
| | (SEQ ID NO: 46) | (SEQ ID NO: 87) | (SEQ ID NO: 683) | |
| | SEHNR | FQNEAQ | ASSPRVPGQGTAGNTIY | 3 |
| | (SEQ ID NO: 56) | (SEQ ID NO: 117) | (SEQ ID NO: 684) | |
| | SEHNR | FQNEAQ | ASSLSVGSGELF | 3 |
| | (SEQ ID NO: 56) | (SEQ ID NO: 117) | (SEQ ID NO: 685) | |
| | MNHEY | SVGAGI | ASSVYFGQPQH | 3 |
| | (SEQ ID NO: 42) | (SEQ ID NO: 88) | (SEQ ID NO: 686) | |
| | MNHEY | SVGAGI | ASSLYFGQTQH | 3 |
| | (SEQ ID NO: 42) | (SEQ ID NO: 88) | (SEQ ID NO: 687) | |
| | MNHEY | SVGAGI | ASSLYFGQAQH | 3 |
| | (SEQ ID NO: 42) | (SEQ ID NO: 88) | (SEQ ID NO: 688) | |
| | MNHEY | SVGAGI | ASSLYFGKPQH | 3 |
| | (SEQ ID NO: 42) | (SEQ ID NO: 88) | (SEQ ID NO: 689) | |
| | MNHEY | SVGAGI | ASRLYFGQPQH | 3 |
| | (SEQ ID NO: 42) | (SEQ ID NO: 88) | (SEQ ID NO: 690) | |
| | MNHEY | SMNVEV | ASSLYWVDTQY | 3 |
| | (SEQ ID NO: 42) | (SEQ ID NO: 115) | (SEQ ID NO: 691) | |
| | MNHEY | SMNVEV | ASSLAYTSTEAF | 3 |
| | (SEQ ID NO: 42) | (SEQ ID NO: 115) | (SEQ ID NO: 692) | |
| | MNHEY | SMDVEV | ASSLYQGPNEQF | 3 |
| | (SEQ ID NO: 42) | (SEQ ID NO: 115) | (SEQ ID NO: 693) | |
| | LNHDA | SQIVND | ASSFRQWAGGGTDTQY | 3 |
| | (SEQ ID NO: 47) | (SEQ ID NO: 109) | (SEQ ID NO: 694) | |
| | SGHRS | YFSETQ | ASSLVQGTWYEQY | 2 |
| | (SEQ ID NO: 59) | (SEQ ID NO: 124) | (SEQ ID NO: 695) | |
| | SGHAT | FQNNGV | ASSLVGGAYNEQF | 2 |
| | (SEQ ID NO: 46) | (SEQ ID NO: 108) | (SEQ ID NO: 696) | |
| | SGHAT | FQDESV | ASSWDRGMKL | 2 |
| | (SEQ ID NO: 46) | (SEQ ID NO: 87) | (SEQ ID NO: 697) | |
| | SGHAT | FQDESV | ASSLVSGGNEQF | 2 |
| | (SEQ ID NO: 46) | (SEQ ID NO: 87) | (SEQ ID NO: 698) | |
| | SGHAT | FQDESV | ASSLGQGNQAF | 2 |
| | (SEQ ID NO: 46) | (SEQ ID NO: 87) | (SEQ ID NO: 699) | |
| | SGHAT | FQDESV | ASSLGQGNEV | 2 |
| | (SEQ ID NO: 46) | (SEQ ID NO: 87) | (SEQ ID NO: 700) | |
| | SGHAT | FQDESV | ASSLGQGDKAF | 2 |
| | (SEQ ID NO: 46) | (SEQ ID NO: 87) | (SEQ ID NO: 701) | |
| | SGHAT | FQDESV | ASGSGQGNEAF | 2 |
| | (SEQ ID NO: 46) | (SEQ ID NO: 87) | (SEQ ID NO: 702) | |
| | PRHDT | FYEKMQ | ASSSLLASGLHTQY | 2 |
| | (SEQ ID NO: 61) | (SEQ ID NO: 126) | (SEQ ID NO: 703) | |
| | MNHNS | SASEGT | ASSPGWRGNSYEQY | 2 |
| | (SEQ ID NO: 53) | (SEQ ID NO: 92) | (SEQ ID NO: 704) | |
| | MNHEY | SVGAGI | ASSLYSGQPS | 2 |
| | (SEQ ID NO: 42) | (SEQ ID NO: 88) | (SEQ ID NO: 705) | |
| | MNHEY | SVGAGI | ASSLYFGEPQH | 2 |
| | (SEQ ID NO: 42) | (SEQ ID NO: 88) | (SEQ ID NO: 706) | |
| | LGHDT | FNNKEL | ASSQLGQGAGEQY | 2 |
| | (SEQ ID NO: 51) | (SEQ ID NO: 128) | (SEQ ID NO: 707) | |
| | DFQATT | SNEGSRA | SARGGSGNEQF | 2 |
| | (SEQ ID NO: 39) | (SEQ ID NO: 129) | (SEQ ID NO: 708) | |
| | DFQATT | SNEGSKA | SAREGSGDEQF | 2 |
| | (SEQ ID NO: 39) | (SEQ ID NO: 89) | (SEQ ID NO: 709) | |

We synthesize peptides and test their immunogenicity in vitro by peptide-binding assays. Using positive and negative controls, we select a set of peptides containing EGFR or Ras mutations that can be presented with certain HLA alleles and treat the patients' tumor cells in vitro. The clonal cells that respond to these peptides are expanded. Subsequently, we use fluorescence-activated cell sorting to isolate CD8+ T cells from the treated and expanded human cells. The CTLs that responded to peptide stimulation are then genetically induced to yield iPS cells. These iPS cells are cloned and redifferentiated to T-iPS cells with incorporation of the iCas9 safety switch. Further, these rejCTL cells are expanded in vitro and used to treat xenografts in mice.

### Example 5

### Safeguard Technology

The tumorigenic potential of undifferentiated iPSCs is a safety concern that must be addressed before iPS cell-based therapies can be routinely used in clinical settings. Using a mouse model, we recently established a way to manipulate a naturally existing suicide pathway to control whether such cells and their progeny live or die. We found that introducing into the cytotoxic T cells a gene encoding a protein called inducible caspase-9, or iC9, permitted us to trigger these cells, and not others, to die throughout the body by activating iC9 with a specific chemical, CID. These engineered T cells still recognize the same antigens, and are just as effective against cancer tumors as are their unmodified peers¹. But they can be quickly eliminated with a simple treatment. This is the first time that a “safeguard system” has been incorporated into in vivo cell-based therapy.

We use this safeguard technology to generate rejuvenated T-iPS cells from lung cancer patients' tissue, blood, or malignant effusion fluid that contain lung cancer specific antigens. In particular, we use specific antigens of the HLA-specific peptide sequences containing alterations in the EGFR protein around mutations (described above). We select clonal T cells that react with these antigens and reprogram them to monoclonal TCR-expressing T-iPSCs with rejuvenated progeny (rejCTLs). We assay the variance of antigen reactivity during the processes of TiPS generation and T-cell redifferentiation by demonstrating genomic rearrangements in TCR genes. Furthermore, these rejCTLs are infused into mice that harbor lung cancer xenografts to determine the treatment effects.

### Example 6

### Methods

### Statistics

Statistical analyses is performed using Excel, Prism (Graphpad Software, La Jolla, Calif.), and Statcel 2 (OMS Publishing, Saitama, Japan) programs, applying ANOVA or a paired-sample Student's t-test, with P<0.05 indicative of significance.

### Peptide-Binding Assay

After incubation in culture medium at 26° C. overnight, T2 cells are washed with PBS and suspended in 1 ml Opti-MEM (Invitrogen Life Technologies, Carlsbad, Calif.) with peptide (100 μg/ml), followed by incubation at 26° C. for 3 h and then at 37° C. for 2.5 h. After washing with PBS, HLA expression is measured using a BD FACSCanto II flow cytometer (BD Biosciences, San Jose, Calif.) using a FITC-conjugated HLA-specific monoclonal antibody. Mean fluorescence intensity is analyzed using FlowJo software.

### PBMC Collection and Lung Cancer Tissue Collection

Peripheral blood samples will be collected from lung cancer patients. PBMCs are isolated by density centrifugation and stored frozen in liquid nitrogen until use. Lung cancer tissue is dissociated into primary cancer cells using an established cell isolation protocol with enzymatic digestion to yield single cells. The mixture of cancer cells is then cultured in RPMI-1640 supplemented with 10% FBS.

### Generation of DCs

CD14+ cells are isolated from PBMCs using CD14 microbeads (Miltenyi Biotec GmbH, Bergisch Gladbach, Germany). Immature dendritic cells (DCs) will be generated from CD14+ cells using IL-4 (10 ng/ml; PeproTech, Rocky Hill, N.J.) and granulocyte-macrophage colony-stimulating factor (GM-CSF) (10 ng/ml) in RPMI-1640 supplemented with 10% FBS. Maturation of DCs will be induced by prostaglandin E2 (PGE2) (1 μg/ml) and tumor necrosis factor-α (TNF-α) (10 ng/ml; PeproTech).

### Induction of Peptide-Specific CTLs

CD8+ T cells (2×10⁶ cells/well) will be stimulated with peptide-pulsed (10 μg/ml) 100-Gy-irradiated autologous mature DCs (1×105 cells/well) in RPMI-1640 containing 10% heat-inactivated human AB serum. After 1 week, these cells will be stimulated twice weekly with peptide-pulsed (10 μg/ml) 200-Gyirradiated aAPC-A2 cells (1×10⁵ cells/well). Supplementation with 10 IU/ml IL-2 and 10 ng/ml IL-15 (PeproTech) are performed at 3- to 4-day intervals between stimulations.

### IFN-γELISPOT Assay

Specific secretion of interferon-y (IFN-γ) from human CTLs in response to stimulator cells are assayed using the IFN-γ enzyme-linked immuno spot (ELISPOT) kit (BD Biosciences, San Jose, Calif.), according to the manufacturer's instructions. Stimulator cells are pulsed with peptide for 2 h at room temperature and then washed three times. Responder cells will be incubated with stimulator cells for 20 h. The resulting spots are counted.

### Cytotoxicity Assay

Cytotoxic capacity are analyzed using the Terascan VPC system (Minerva Tech, Canada). The CTL line is used as the effector cell type. Target cells are labeled in calcein-AM solution for 30 min at 37° C. The labeled cells will then be co-cultured with the effector cells for 4-6 h. Fluorescence intensity will be measured before and after the culture period, and specific cytotoxic activity will be calculated using the following formula: % cytotoxicity={1−[(average fluorescence of the sample wells−average fluorescence of the maximal release control wells)/(average fluorescence of the minimal release control wells−average fluorescence of the maximal release control wells)]}×100.

### Preparation of T-Cells, Infection, and T-iPS Generation

T cells will be isolated by gating the CD3+CD56− population to avoid contamination by natural killer T cells. T-cell subsets will be separated, using additional gating strategies, into CD4 (CD4+CD8−) and CD8 (CD4CD8+) cohorts. CD4 and/or CD8 cells are further classed as naïve (CD45RA+CD62L+), central memory (CD45RA+CD62L−), effector memory (CD45RA-CD62L−), or terminal effector (CD45RA-CD62L+). Sorted cells, initially cultured in Roswell Park Memorial Institute RPMImedium (GIBCO-Invitrogen, Carlsbad, Calif.) supplemented with 10% fetal bovine serum (GIBCO-Invitrogen), 100 U/ml penicillin, 100 ng/ml streptomycin, 2 mM L-glutamine, and 20 ng/ml human interleukin 2 (hIL-2; Novartis Vaccines & Diagnostics, Emeryville, Calif.), are activated by anti-CD3/CD28-conjugated magnetic beads (Dynabeads® ClinExVivo™ CD3/CD28; Invitrogen) at a 3:1 bead:T cell ratio. We define the date of activation as day 0 in the process of T-iPS cell generation.

In some experiments, magnetically captured CD3+ cells are separated from PBMNCs and stimulated concurrently with anti-CD3/CD28-conjugated magnetic beads. At days 6 and 7, the cells are infected with sendai virus vector carrying iPS-reprogramming factors. Medium for primary T-cell culture are changed every day. At day 8, infected cells are collected and transferred onto irradiated MEF layers at 3×10⁵ cells per 6-cm dish. For 4 days thereafter, half-volumes of culture medium are daily replaced with Dulbecco's modified Eagle medium/F12 medium supplemented with 20% Knockout Serum Replacement (GIBCO-Invitrogen), 200 μM L-glutamine (Invitrogen), 1% non-essential amino acids, 10 μM 2-mercaptoethanol, and 5 ng/ml b-FGF as described (“human iPSC medium”). VPA are added at 0.5 mM to human iPS medium before picking up iPSC colonies. At day 12, the entire volume of medium is changed to human iPS medium containing VPA. When human ES/iPS-like colonies become identifiable, around day 21, they are mechanically isolated and dissociated into small clamps by pipetting, with reseeding onto fresh MEF layers. Human ES/iPS-like clones are passed onto new MEF layers every 6 days using trypsin solution (0.25% trypsin, 1 mM CaCl₂), and 20% Knockout Serum Replacement in PBS).

### Karyotyping

Chromosomal G-band analyses are conducted in routine fashion (Nihon Gene Research Laboratories, Miyagi, Japan).

### Alkaline Phosphatase Staining and Immunocytochemistry

Human iPS-like colonies fixed in ice-cold fixative solution (90% methanol, 10% formaldehyde) will bestained using a kit (Vector Laboratories, Burlingame, Calif.) according to manufacturer's instructions. For immunocytochemical staining, human iPS-like colonies fixed in 5% paraformaldehyde are permeabilized with 0.1% Triton X-100. The pretreated colonies are incubated first with primary antibodies (PE-conjugated anti-SSEA-4, 1:50, FAB1435P, R&D Systems, Minneapolis, Minn.; anti-TRA-160, 1:100, MAB4360, Millipore, Billerica; or anti-TRA-1-81, 1:100, MAB4381, Millipore). The secondary antibody used for TRA-1-60 and TRA-1-81 Will be Alexa Fluor 488-conjugated goat anti-mouse antibody (1:500; A11029, Molecular Probes-Invitrogen). Nuclei are counterstained with DAPI; 1:1000 (Roche Diagnostics, Indianapolis, Ind.). Photographs will be taken using a fluorescence microscope.

### Teratoma Formation

Human iPS-like colonies are clumped and injected (1.0×10⁶ cells/mouse) into the medulla of the left testis of NOD-SCID mice. Eight weeks after injection, tumors formed in the testis are resected, fixed in 5% paraformaldehyde, and embedded in paraffin. Sections are stained with hematoxylin/eosin technique and examined by light microscopy for evidence of tri-lineage germ layer differentiation.

### Pluripotent Genes and T-Cell-Related Genes Expression Analysis

Using an RNeasy mini kit (Qiagen, Hilden, Germany), total RNA will be extracted from iPS cells (about 50 days after cloning), their progeny cells, and freshly isolated peripheral-blood CD3 T-cells. Total RNA (1 μg) are reverse transcribed with a PrimeScript III cDNA Synthesis Kit (Invitrogen). PCRs are performed using ExTaq HS (Takara) at 30 cycles for housekeeping genes (GAPDH or ACTB) and at 35 cycles for all pluripotent or T-cell related genes.

### Detecting TCR Rearrangement in Genomic DNA of T-iPS Cells

Genomic DNA are extracted from approximately 5×10⁶ T-iPS cells using QIAamp DNA kits (Qiagen). Extracted DNA (40 ng) are used in each PCR to detect TCRG, TCRB and TCRA gene rearrangements. PCRs for detecting TCRG rearrangement are performed. The V, D, and J segments involved in assembled TCRA or TCRB are identified by comparison with published sequences and with the ImMunoGeneTics (IMGT) database (cines.fr/), as well as by using web tools such as v-quest. Gene-segment nomenclature follows IMGT usage.

Induction of T-Lineage Cells from T-iPS Cells

Briefly, iPS cells are co-cultured on an irradiated OP9 layer for 10 to 14 days in DMEM medium without cytokines. Floating cells packed and transferred onto OP9-DL1 layers (day 0), are co-cultured in αMEM-based medium supplemented with 10 ng/ml of hIL-7 and hFlt-3L for up to day 28. The culture medium is changed every 3 days. T-lineage cells, floating above OP9-DL1 layers and expressing CD45, CD3, and TCR, are sorted by flow cytometry weekly and gene expression analyses will be carried out.

### Antitumor Activity in In Vivo Model

Treatment efficacy is evaluated in SCID mice engrafted with patient NSCLCs. To evaluate the antitumor effects of rejT-iC9-CTLs in SCID mice engrafted with lung cancer, tumor growth is monitored using a bioluminescence system. Once a progressive increase of bioluminescence occurs, mice are treated intraperitoneally with 3 once weekly doses of rejT-iC9-CTL and control CTLs (10×10⁶ CTL/mouse). Tumor burden is monitored by the Xenogen-IVIS imaging system. Mice are injected intraperitoneally with d-luciferin (150 mg/kg) and light output is analyzed using the Xenogen Living Image Software Version 2.50 (Xenogen, Alameda, Calif.).

In Vivo Elimination of iC9-iPSC-Derived CTLs

To examine whether iC9-iPSC-derived CTLs can be eliminated by this iC9/CID safeguard system in vivo, SCID mice engrafted with lung cancer tissues are treated with CID (50 μg i.p. daily for three successive days (day 2-day 4). Comparison mice will not receive CID.

Detection of rejT-iC9-CTLs in vivo

SCID mice inoculated intraperitoneally with iC9-iPSC-derived CTLs labeled with GFP/FFluc are treated with 10×10⁶ rejT-iC9− cells on day 0 and day 7. After rejT-iC9− cells are detected in peripheral blood (around 8 days after first rejT-iC9 administration), the mice receive intraperitoneally injected CID, at 50 ug/mouse, for three successive days. Control mice I receive three doses of PBS. Flow cytometry of peripheral blood is used to identify rejT-iC9-cells (expressing mCherry).

Establishment of NSCLC Cell Lines and Tissues Engraftable into Immunodeficient Mice

Lung cancer tissue samples are collected from surgically resected tumors. We have an established lung cancer tissue dissociation protocol that can be used to precede primary culture. Whole blood is processed for T cell culture and used for the establishment of NSCLC-specific CTLs. Patient tumor tissue or cells in malignant effusions from cancer patients are used to establish xenografts in SCID mice. Briefly, patient's tumor chunks or malignant-effusion cells are transplanted into SCID mice. Tumor size is measured in these mice to assess the progress of lung cancer in this in vivo model. SCID mice successfully engrafted with tumor will be used for further treatments.

### REFERENCES

1. Nishimura, T., Kaneko, S., Kawana-Tachikawa, A., Tajima, Y., Goto, H., Zhu, D., NakayamaHosoya, K., Iriguchi, S., Uemura, Y., Shimizu, T., et al. 2013. Generation of rejuvenated antigen-specific T cells by reprogramming to pluripotency and redifferentiation. Cell Stem Cell 12:114-126.
2. Vodyanik, M. A., Bork, J. A., Thomson, J. A., and Slukvin, I I. 2005. Human embryonic stem cellderived CD34+ cells: efficient production in the coculture with OP9 stromal cells and analysis of lymphohematopoietic potential. Blood 105:617-626.
3. Takayama, N., Nishikii, H., Usui, J., Tsukui, H., Sawaguchi, A., Hiroyama, T., Eto, K., and Nakauchi, H. 2008. Generation of functional platelets from human embryonic stem cells in vitro via ES-sacs, VEGF-promoted structures that concentrate hematopoietic progenitors. Blood 111:5298-5306.
4. Schmitt, T. M., and Zuniga-Pflucker, J. C. 2002. Induction of T cell development from hematopoietic progenitor cells by delta-like-1 in vitro. Immunity 17:749-756.
5. La Motte-Mohs, R. N., Herer, E., and Zuniga-Pflucker, J. C. 2005. Induction of T-cell development from human cord blood hematopoietic stem cells by Delta-like 1 in vitro. Blood 105:1431-1439.
6. Vizcardo, R., Masuda, K., Yamada, D., Ikawa, T., Shimizu, K., Fujii, S., Koseki, H., and Kawamoto, H. 2013. Regeneration of human tumor antigen-specific T cells from iPSCs derived from mature CD8(+) T cells. Cell Stem Cell 12:31-36.
7. Chen, G., Gulbranson, D. R., Hou, Z., Bolin, J. M., Ruotti, V., Probasco, M. D., Smuga-Otto, K., Howden, S. E., Diol, N. R., Propson, N. E., et al. 2011. Chemically defined conditions for human iPSC derivation and culture. Nat Methods 8:424-429.
8. Ando M, Nishimura T, Yamazaki S, Yamaguchi T, Kawana-Tachikawa A, Hayama T, Nakauchi Y, Ando J, Ota Y, Takahashi S, Nishimura K, Ohtaka M, Nakanishi M, Miles J J, Burrows S R, Brenner M K, Nakauchi H. A Safeguard System for Induced Pluripotent Stem Cell-Derived Rejuvenated T Cell Therapy 2015 Oct. 13; 5(4):597-608. Stem Cell Report
9. Yagyu S, Hoyos V, Del Bufalo F, Brenner M K 2015 An Inducible Caspase-9 Suicide Gene to Improve the Safety of Therapy Using Human Induced Pluripotent Stem Cells. Mol Ther. 2015 September; 23(9):1475-85
10. Lefranc, M. P. 2003. IMGT databases, web resources and tools for immunoglobulin and T cell receptor sequence analysis, http://imgt.cines.fr. Leukemia 17:260-266.
11. Yamada T, Azuma K, Muta E, Kim J, Sugawara S, Zhang G L, Matsueda S, KasamaKawaguchi Y, Yamashita Y, Yamashita T, Nishio K, Itoh K, Hoshino T, Sasada T. EGFR T790M mutation as a possible target for immunotherapy; identification of HLA-A*0201restricted T cell epitopes derived from the EGFR T790M mutation. PLoS One. 2013 Nov. 5; 8(11)
12. Ofuji K, Tada Y, Yoshikawa T, Shimomura M, Yoshimura M, Saito K, Nakamoto Y, Nakatsura T. A peptide antigen derived from EGFR T790M is immunogenic in non-small cell lung cancer. Int J Oncol. 2015 February; 46(2):497-504
13. NCCN Clinical Practice Guidelines in Oncology™. Non-small cell lung cancer. v 2.2010. Available at: http://www.nccn.org/professionals/physician_gls/f_guidelines.asp. Accessed Jul. 22, 2010
14. West H, Lilenbaum R, Harpole D, et al. Molecular analysis-based treatment strategies for the management of non-small cell lung cancer. J Thorac Oncol. 2009; 4:s1029-s1039.
15. Jackman D M, Miller V A, Cioffredi L A, et al. Impact of epidermal growth factor receptor and KRAS mutations on clinical outcomes in previously untreated non-small cell lung cancer patients: Results of an online tumor registry of clinical trials. Clin Cancer Res. 2009; 15:5267-5273
16. Klebanoff C A, Rosenberg S A, Restifo N P Prospects for gene-engineered T cell immunotherapy for solid cancers. Nat Med. 2016 Jan. 6; 22(1):26-3
17. Skora A D, Douglass J, Hwang M S, Tam A J, Blosser R L, Gabelli S B, Cao J, Diaz L A Jr, Papadopoulos N, Kinzler K W, Vogelstein B, Zhou S Generation of MANAbodies specific to HLArestricted epitopes encoded by somatically mutated genes. Proc Natl Acad Sci USA. 2015 Aug. 11; 112(32):9967-72
18. Morello A, Sadelain M, Adusumilli P S, Mesothelin-Targeted CARs: Driving T Cells to Solid Tumors Cancer Discov. 2016 February; 6(2):133-46
19. Adusumilli P S, Cherkassky L, Villena-Vargas J, Colovos C, Servais E, Plotkin J, Jones D R, Sadelain M. Regional delivery of mesothelin-targeted CAR T cell therapy generates potent and long-lasting CD4-dependent tumor immunity. Sci Transl Med. 2014 Nov. 5; 6(261):261ra151
20. Beatty G L, Haas A R, Maus M V, Torigian D A, Soulen M C, Plesa G, Chew A, Zhao Y, Levine B L, Albelda S M, Kalos M, June C H, Mesothelin-specific chimeric antigen receptor mRNA-engineered T cells induce anti-tumor activity in solid malignancies Cancer Immunol Res. 2014 February; 2(2):11220
21. Gao X, Le X, Costa D B, The safety and efficacy of osimertinib for the treatment of EGFR T790M mutation positive non-small-cell lung cancer Expert Rev Anticancer Ther. 2016 Mar. 4
22. Noda S, Kanda S Addressing epidermal growth factor receptor tyrosine kinase inhibitor resistance in non-small cell lung cancer Expert Rev Respir Med. 2016 Mar. 9
23. van der Wekken A J, Saber A, Hiltermann T J, Kok K, van den Berg A, Groen H J. Resistance mechanisms after tyrosine kinase inhibitors afatinib and crizotinib in non-small cell lung cancer, a review of the literature Crit Rev Oncol Hematol. 2016 Jan. 25
24. Villadolid J, Ersek J L, Fong M K, Sirianno L, Story E S Management of hyperglycemia from epidermal growth factor receptor (EGFR) tyrosine kinase inhibitors (TKIs) targeting T790Mmediated resistance Transl Lung Cancer Res. 2015 October; 4(5):576-83
25. Black R C, Khurshid H NSCLC: An Update of Driver Mutations, Their Role in Pathogenesis and Clinical Significance R I Med J (2013). 2015 Oct. 1; 98(10):25-8
26. Berman A T, Simone C B 2nd Immunotherapy in locally-advanced non-small cell lung cancer: releasing the brakes on consolidation? Transl Lung Cancer Res. 2016 February; 5(1):138-42
27. Ahmad S M, Borch T H, Hansen M, Andersen M H PD-L1-specific T cells Cancer Immunol Immunother. 2016 Jan. 2
28. Zhukovsky E A, Morse R J, Maus M V Bispecific antibodies and CARs: generalized immunotherapeutics harnessing T cell redirection Curr Opin Immunol. 2016 Mar. 7; 40:24-35
29. Simon S, Vignard V, Florenceau L, Dreno B, Khammari A, Lang F, Labarriere N PD-1 expression conditions T cell avidity within an antigen-specific repertoire Oncoimmunology. 2015 Oct. 29; 5(1)
30. Hasegawa K, Tanaka S, Fujiki F, Morimoto S, Nakajima H, Tatsumi N, Nakata J, Takashima S, Nishida S, Tsuboi A, Oka Y, Oji Y, Kumanogoh A, Sugiyama H, Hosen N An Immunocompetent Mouse Model for MLL/AF9 Leukemia Reveals the Potential of Spontaneous Cytotoxic T-Cell Response to an Antigen Expressed in Leukemia Cells PLoS One. 2015 Dec. 11; 10(12)
31. Zou W, Wolchok J D, Chen L PD-L1 (B7-H1) and PD-1 pathway blockade for cancer therapy: Mechanisms, response biomarkers, and combinations Sci Transl Med. 2016 Mar. 2; 8(328):328
32. Naidoo J, Schindler K, Querfeld C, Busam K J, Cunningham J, Page D B, Postow M A, Weinstein A, Skripnik Lucas A, Ciccolini K T, Quigley E A, Lesokhin A M, Paik P K, Chaft J E, Segal N H, D'Angelo S P, Dickson M A, Wolchok J D, Lacouture M E Autoimmune Bullous Skin Disorders with Immune Checkpoint Inhibitors Targeting PD-1 and PD-L1 Cancer Immunol Res. 2016 Feb. 29

1. A method of cellular immunotherapy for treating a subject for a cancer expressing a mutated epidermal growth factor receptor (EGFR) or KRAS neo-antigen epitope, the method comprising:
a) eliciting an antigen-specific cytotoxic T cell response by contacting cytotoxic T cells (CTLs) with an antigen presenting cell presenting at its surface an immunogenic peptide comprising the mutated EGFR or KRAS neo-antigen epitope in a complex with major histocompatibility complex (MHC);
b) isolating CTLs specific for the mutated EGFR or KRAS neo-antigen epitope;
c) generating induced pluripotent stem cells (IPSC) from the CTLs specific for the mutated EGFR or KRAS neo-antigen epitope;
d) differentiating the IPSCs into rejuvenated CTLs specific for the mutated EGFR or KRAS neo-antigen epitope; and
e) administering a therapeutically effective amount of the rejuvenated CTLs specific for the mutated EGFR or KRAS neo-antigen epitope to the subject.

2. The method of claim 1, wherein the mutated EGFR neo-antigen epitope comprises a mutation selected from the group consisting of a C797S mutation, a T790M mutation, an L858R mutation, and a deletion, or the mutated KRAS neo-antigen comprises a mutation selected from the group consisting of a G12D mutation, a G12V mutation, and a G12C mutation.

3. (canceled)

4. The method of claim 1, wherein the immunogenic peptide is selected from the group consisting of:
a) an immunogenic peptide comprising an amino acid sequence selected from the group consisting of SEQ ID NOS:1-5; and
b) an immunogenic peptide comprising an amino acid sequence having at least 70% identity to an amino acid sequence selected from the group consisting of SEQ ID NOS:1-5, wherein the immunogenic peptide comprises the mutated EGFR or KRAS neo-antigen epitope.

5. (canceled)

6. The method of claim 1, wherein the CTLs are contacted with the antigen presenting cell in vivo, ex vivo, or in vitro.

7. The method of claim 1, wherein the CTLs specific for the mutated EGFR or KRAS neo-antigen epitope are isolated from tumor infiltrating lymphocytes or peripheral blood mononuclear cells.

8. The method of claim 1, wherein the CTLs are provided in a biological sample, wherein the biological sample is blood, a tumor biopsy, a cancerous tissue sample, or a malignant effusion fluid sample.

9-10. (canceled)

11. The method of claim 1, wherein the CTLs are autologous or allogeneic.

12. The method of claim 1, wherein the CTLS are obtained from a donor that is human leukocyte antigen (HLA)-matched with the subject.

13. The method of claim 1, wherein the rejuvenated CTLs express CD8.

14. The method of claim 1, wherein the rejuvenated CTLs are expanded in vitro before being administered to the subject.

15-16. (canceled)

17. The method of claim 1, wherein multiple cycles of treatment are administered to the subject for a time period sufficient to effect at least a partial tumor response or a complete tumor response.

18. (canceled)

19. The method of claim 1, wherein the cancer expresses a major histocompatibility complex (MHC) carrying a peptide comprising the mutated EGFR or KRAS neo-antigen epitope.

20. The method of claim 1, further comprising introducing a suicide gene into the rejuvenated CTLs.

21-22. (canceled)

23. The method of claim 1, wherein the antigen presenting cell is a dendritic cell, a macrophage, an artificial antigen presenting cell, or a cancerous cell expressing the mutated epidermal growth factor receptor (EGFR) or KRAS neo-antigen epitope.

24-29. (canceled)

30. A method of producing an induced pluripotent stem cell (IPSC)-derived rejuvenated cytotoxic T cell (CTL) specific for a mutated EGFR or KRAS neo-antigen epitope, the method comprising:
a) obtaining a biological sample comprising cytotoxic T cells (CTLs);
b) eliciting an antigen-specific cytotoxic T cell response by contacting cytotoxic T cells (CTLs) with an antigen presenting cell presenting at its surface an immunogenic peptide comprising a mutated EGFR or KRAS neo-antigen epitope in a complex with major histocompatibility complex;
c) isolating a CTL specific for the mutated EGFR or KRAS neo-antigen epitope;
d) generating an induced pluripotent stem cell (IPSC) from the CTL specific for the mutated EGFR or KRAS neo-antigen epitope; and
e) differentiating the IPSC into a rejuvenated CTL specific for the mutated EGFR or KRAS neo-antigen epitope.

31. The method of claim 30, wherein the mutated EGFR neo-antigen comprises a C797S mutation, a T790M mutation, an L858R mutation, or a deletion, or the mutated KRAS neo-antigen comprises a mutation selected from the group consisting of a G12D mutation, a G12V mutation, and a G12C mutation.

32. (canceled)

33. The method of claim 30, wherein the immunogenic peptide is selected from the group consisting of:
a) an immunogenic peptide comprising an amino acid sequence selected from the group consisting of SEQ ID NOS:1-5; and
b) an immunogenic peptide comprising an amino acid sequence having at least 70% identity to an amino acid sequence selected from the group consisting of SEQ ID NOS:1-5, wherein the immunogenic peptide comprises the mutated EGFR or KRAS neo-antigen epitope.

34. The method of claim 30, wherein the biological sample is blood, a tumor biopsy, a cancerous tissue sample, or a malignant effusion fluid sample.

35-37. (canceled)

38. An IPSC-derived rejuvenated CTL produced according to the method of claim 30.

39. A composition comprising the IPSC-derived rejuvenated CTL of claim 38 and a pharmaceutically acceptable excipient.

40-51. (canceled)
